(19) United States
(12) Patent Application Publication
(10) Pub. No.: US 2007/0093483 A1
(43) Pub. Date: Apr. 26, 2007

(54) Use of derivatives of 2, 4-dihydro-[1,2,4] triazole-3-thione as inhibitors of the enzyme myeloperoxidase (mpo)

(75) Inventors: Mats Svensson, Sodertalje (SE); Anna-Karin Tiden, Sodertalje (SE); Dominika Turek, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 10/554,659

(22) PCT Filed: Apr. 22, 2004
(86) PCT No.: PCT/SE04/00618

§ 371 Date: Oct. 18, 2006

(30) Foreign Application Priority Data
Apr. 25, 2003 (SE) 0301232-5

(51) Int. Cl. A61K 31/5377; A61K 31/433; A61K 31/42; A61K 31/4196; C07D 417/02; C07D 405/02; C07D 261/18; C07D 249/12

(52) U.S. Cl. 514/235.5; 514/384; 548/263.2; 514/378; 514/362; 548/248; 548/147; 544/132

(57) ABSTRACT

There is disclosed the use of a compound of formula (I) wherein X, Y, W and Q are as defined in the specification, and pharmaceutically acceptable salts thereof, in the manufacture of a medicament, for the treatment or prophylaxis of diseases or conditions in which inhibition of the enzyme myeloperoxidase (MPO) is beneficial. Certain novel compounds of formula (I) and pharmaceutically acceptable salts thereof are disclosed, together with processes for their preparation. The compounds of formulae (I) are MPO inhibitors and are thereby particularly useful in the treatment or prophylaxis of neuroinflammatory disorders.

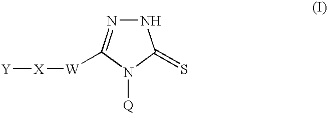

### FIELD OF THE INVENTION

The present invention relates to the use of derivatives of 2,4dihydro-[1,2,4]triazole-3-thione as inhibitors of the enzyme myeloperoxidase (NPO). Certain novel 2,4-dihydro-[1,2,4]triazole-3-thione derivatives are also disclosed together with processes for their preparation, compositions containing them and their use in therapy.

### BACKGROUND OF THE INVENTION

Myeloperoxidase (MPO) is a heme-containing enzyme found predominantly in polymorphonuclear leukocytes (PMNs). MPO is one member of a diverse protein family of mammalian peroxidases that also includes eosinophil peroxidase, thyroid peroxidase, salivary peroxidase, lactoperoxidase, prostaglandin H synthase, and others. The mature enzyme is a dimer of identical halves. Each half molecule contains a covalently bound heme that exhibits unusual spectral properties responsible for the characteristic green colour of MPO. Cleavage of the disulphide bridge linking the two halves of MPO yields the hemi-enzyme that exhibits spectral and catalytic properties indistinguishable from those of the intact enzyme. The enzyme uses hydrogen peroxide to oxidize chloride to hypochlorous acid. Other halides and pseudohalides (like thiocyanate) are also physiological substrates to MPO.

PMNs are of particular importance for combating infections. These cells contain MPO, with well documented microbicidal action. PMNs act non-specifically by phagocytosis to engulf microorganisms, incorporate them into vacuoles, termed phagosomes, which fuse with granules containing myeloperoxidase to form phagolysosomes. In phagolysosomes the enzymatic activity of the myeloperoxidase leads to the formation of hypochlorous acid, a potent bactericidal compound. Hypochlorous acid is oxidizing in itself, and reacts most avidly with thiols and thioethers, but also converts amines into chloramines, and chlorinates aromatic amino acids. Macrophages are large phagocytic cells which, like PMNs, are capable of phagocytosing microorganisms. Macrophages can generate hydrogen peroxide and upon activation also produce myeloperoxidase. MPO and hydrogen peroxide can also be released to the outside of the cells where the reaction with chloride can induce damage to adjacent tissue.

Linkage of myeloperoxidase activity to disease has been implicated in neurological diseases with a neuroinflammatory response including multiple sclerosis, Alzheimer's disease, Parkinson's disease and stroke as well as other inflammatory diseases or conditions like asthma, chronic obstructive pulmonary disease, cystic fibrosis, atherosclerosis, inflammatory bowel disease, renal glomerular damage and rheumatoid arthritis. Lung cancer has also been suggested to be associated with high MPO levels.

WO 01/85146 discloses various compounds that are MPO inhibitors and are thereby useful in the treatment of chronic obstructive pulmonary disease (COPD).

The present invention relates to a group of 2,4-dihydro-[1,2,4]triazole-3-thione derivatives that surprisingly display useful properties as inhibitors of the enzyme MPO.

### DISCLOSURE OF THE INVENTION

According to the present invention, there is provided the use of a compound of formula (I) 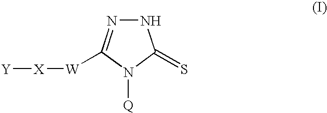
wherein:
Q represents phenyl, naphthyl or a monocyclic or bicyclic heteroaromatic ring system containing one to three heteroatoms independently selected from O, N and S; said phenyl, naphthyl or heteroaromatic ring being optionally substituted by one to three substituents independently selected from halogen, CN, C1 to 6 alkyl, C1 to 6 alkoxy, C1 to 6 alkylthio, CO₂R⁶, COR⁷, CH₂OH, Ph, NO₂, NR⁸R⁹ and SO₂NR¹⁰R¹¹; said alkyl or alkoxy group being optionally further substituted by one or more fluoro atoms;
or Q represents C1 to 6 alkyl optionally substituted by one or more groups independently selected from C1 to 6 alkoxy, NR⁸R⁹, phenyl, a 5- or 6-membered heteroaromatic ring containing one or two heteroatoms independently selected from O, S and N, or a 5- or 6-membered saturated heterocyclic ring containing one or two heteroatoms independently selected from O, N and S;
or Q represents C3 to 8 cycloalkyl;
W represents a bond or CHR¹ wherein R¹ represents H, CH₃, F, OH, CH₂OH or Ph;
X represents a bond, O, CH₂ or NR³ wherein R³ represents H or C1 to 6 alkyl;
Y represents phenyl, naphthyl or a monocyclic or bicyclic heteroaromatic ring system containing one to three heteroatoms independently selected from O, N and S; said phenyl, naphthyl or heteroaromatic ring system being optionally substituted by one to three substituents independently selected from halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
or Y represents C1 to 6 alkyl or C3 to 6 cycloalkyl; said cycloalkyl group optionally including an O atom and optionally being benzo fused; and said alkyl or cycloalkyl group being optionally substituted by one or more substituents independently selected from halogen, oxo (═O), C1 to 6 alkyl or C1 to 6 alkoxy;
each R⁴, R⁵, R⁶, R⁷, R¹² and R¹³ independently represents H or C1 to 6 alkyl;
each R⁸, R⁹, R¹⁰ and R¹¹ independently represents H or C1 to 6 alkyl; or the group NR⁸R⁹ or NR¹⁰R¹¹ together represents a saturated 5- or 6-membered azacyclic ring optionally including one further heteroatom selected from O, S and N, and optionally being substituted by one or more C1 to 6 alkyl groups;
or a pharmaceutically acceptable salt thereof, in the manufacture of a medicament, for the treatment or prophylaxis of diseases or conditions in which inhibition of the enzyme MPO is beneficial.

The compounds of formula (I) may exist in enantiomeric forms. Therefore, all enantiomers, diastereomers, racemates and mixtures thereof are included within the scope of the invention.

The compounds of formula (I) may exist in tautomeric forms. All such tautomers and mixtures of tautomers are included within the scope of the present invention.

A more particular aspect of the invention provides the use of a compound of formula (I), or a pharmaceutically acceptable salt thereof, in the manufacture of a medicament, for the treatment or prophylaxis of neuroinflammatory disorders.

According to the invention, there is also provided a method of treating, or reducing the risk of, diseases or conditions in which inhibition of the enzyme MPO is beneficial which comprises administering to a person suffering from or at risk of, said disease or condition, a therapeutically effective amount of a compound of formula (I), or a pharmaceutically acceptable salt thereof.

More particularly, there is also provided a method of treating, or reducing the risk of, neuroinflammatory disorders in a person suffering from or at risk of, said disease or condition, wherein the method comprises administering to the person a therapeutically effective amount of a compound of formula (I), or a pharmaceutically acceptable salt thereof.

In another aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of diseases or conditions in which inhibition of the enzyme MPO is beneficial.

In another more particular aspect the invention provides a pharmaceutical formulation comprising a therapeutically effective amount of a compound of formula (I), or a pharmaceutically acceptable salt thereof, in admixture with a pharmaceutically acceptable adjuvant, diluent or carrier, for use in the treatment or prophylaxis of neuroinflammatory disorders.

In one embodiment, Q in formula (I) represents phenyl optionally substituted by halogen, C1 to 6 alkyl or C1 to 6 alkoxy. In another embodiment, Q in formula (I) represents phenyl optionally substituted by halogen, C1 to 2 alkyl or C1 to 2 alkoxy. In another embodiment, Q in formula (I) represents unsubstituted phenyl.

In one embodiment, W represents a bond or CH₂.

In one embodiment, X represents a bond or O.

In one embodiment, W represents CH₂ and X represents a bond.

In one embodiment, W represents CH₂ and X represents O.

In one embodiment, Y represents phenyl optionally substituted as defined above.

In one embodiment, Q in formula (I) represents phenyl optionally substituted by halogen, C1 to 2 alkyl or C1 to 2 alkoxy; W represents CH₂; X represents O; and Y represents phenyl optionally substituted as defined above.

In one embodiment, Q in formula (I) represents phenyl optionally substituted by halogen, C1 to 2 alkyl or C1 to 2 alkoxy; W represents CH₂; X represents a bond; and Y represents phenyl optionally substituted as defined above.

In one aspect, the invention concerns the use of compounds of formula (I) wherein Q represents phenyl, naphthyl or a 5- or 6-membered heteroaromatic ring containing one or two heteroatoms independently selected from O, S and N; said phenyl, naphthyl or heteroaromatic ring being optionally substituted by one to three substituents independently selected from halogen, C1 to 6 alkyl, C1 to 6 alkoxy, C1 to 6 alkylthio, CO₂R⁶, COR⁷, CH₂OH, NO₂, NR⁸R⁹ and SO₂NR¹⁰R¹¹; said alkyl or alkoxy group being optionally further substituted by one or more fluoro atoms; or Q represents C1 to 6 alkyl optionally substituted by one or more groups independently selected from C1 to 6 alkoxy, NR⁸R, phenyl or a 5- or 6-membered saturated heterocyclic ring containing one or two heteroatoms independently selected from O, N and S; or Q represents C3 to 8 cycloalkyl; W represents a bond or CHR¹ wherein R¹ represents H, CH₃, F, OH, CH₂OH or Ph; X represents a bond, O or NR³ wherein R³ represents H or C1 to 6 alkyl; Y represents phenyl, naphthyl or a monocyclic or bicyclic heteroaromatic ring system containing one to three heteroatoms independently selected from O, N and S; said phenyl, naphthyl or heteroaromatic ring system being optionally substituted by one to three substituents independently selected from halogen, OH, C1 to 6 alkyl, C3 to 6 cycloalkyl, C1 to 6 alkoxy, C1 to 6 alkylthio, CO₂H, NO₂ or NR⁴R⁵; said alkyl, cycloalkyl, alkoxy and alkylthio groups being optionally further substituted by one or more fluoro atoms; or Y represents C1 to 6 alkyl or C3 to 6 cycloalkyl, said alkyl or cycloalkyl group being optionally substituted by halogen, C1 to 6 alkyl or C1 to 6 alkoxy; R⁴, R⁵, R⁶, R⁷, R⁸ and R⁹ independently represent H or C1 to 6 alkyl; and R¹⁰ and R¹¹ independently represent H or C1 to 6 alkyl; or the group NR¹⁰R¹¹ together represents a saturated 5- or 6-membered azacyclic ring.

A specific aspect of the invention concerns the use of any one or more of the following compounds of formula (I):
5-(4-aminobenzyl)-4-[3,5-di(trifluoromethyl)phenyl]-2,4-dihydro-[1,2,4]triazole-3-thione;
5-(4-chlorobenzyl)4(4-methylphenyl)-2,4-dihydro-[1,2,4]triazole-3-thione;
5-isobutyl-4-phenyl-2,4-dihydro-[1,2,4]triazole-3-thione;
5-(4-hydroxybenzyl)4-[3-(methylthio)phenyl]-2,4-dihydro-[1,2,4]triazole-3-thione;
5-(2,5-dimethoxybenzyl)4-[3-(inethylthio)phenyl]-2,4-dihydro-[1,2,4]triazole-3-thione,
5-(4-hydroxybenzyl)4-(4-iodophenyl)-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(4-hydroxybenzyl)-4-[4-(piperidinosulfonyl)phenyl]-2,4-dihydro-[1 ,2,4]triazole-3-thione;
5-(2,5-dimethoxybenzyl)-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
5-(4-hydroxybenzyl)-4-(2,4,6-trichlorophenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
4-(4-carboxyphenyl)-5-(diphenylmethyl)-2,4-dihydro-[1,2,4]triazole-3-thione;
5-(2-bromobenzyl)4-(4-sulfamoylphenyl)-2,4-dihydro-[1,2,4]triazole-3-thione;
5-(4-hydroxybenzyl)-4-(naphthalen-1-yl)-2,4-dihydro-[1,2,4]triazole-3-thione;
5-(4-hydroxybenzyl)-4-(2,6-dibromo-4-methylphenyl)-2,4-dihydro-[1,2,4]triazole-3-thione;
5-(4-hydroxybenzyl)-4-(3,4,5-trimethoxyphenyl)-2,4-dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(4-methoxyphenoxymethyl)-4-(2-tetrahydrofuran-2-yl-methyl)-2,4-dihydro-[1,2,4]triazole-3-thione;
5-(4-methoxyphenoxymethyl)-4-(3-methoxypropyl)-2,4dihydro-[1,2,4]triazole-3-thione;
5-(4-methoxyphenoxymethyl)-4-(2-phenylethyl)-2,4-dihydro-[1,2,4]triazole-3-thione;
5-(4-methoxyphenoxymethyl)-4-(3-morpholin-4-yl-propyl)-2,4-dihydro-[1,2,4]triazole-3-thione;
4-butyl-5-[(4-methoxyphenylamino)-methyl]-2,4-dihydro-[1,2,4]triazole-3-thione;
5-[(4-methoxyphenylamino)-methyl]-4-(3-methoxypropyl)-2,4-dihydro- [1,2,4]triazole-3-thione;
4-hexyl-5-[(4-methoxyphenylamino)methyl]-2,4-dihydro-[1,2,4]triazole-3-thione;
5-(2-chlorobenzyl)-4-(2-ethoxyphenyl)-2,4-dihydro-[1,2,4]triazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-pyridin-3-yl-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(4-methoxyphenoxymethyl)-4-cyclopropyl-2,4-dihydro-[1,2,4]triazole-3-thione;
4-(2,2-dimethoxyethyl)-5-[(4-methoxyphenylamino)methyl]-2,4-dihydro-[1,2,4)triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4-dihydro-[1,2,4]triazole-3-thione;
5-(2-chlorobenzyl)-4-(3-methylphenyl)-2,4-dihydro-[1,2,4]triazole-3-thione;
5-[(2-chlorophenyl)hydroxymethyl]-4-isobutyl-2,4-dihydro-[1,2,4]triazole-3-thione;
5-[(2-chloro-phenyl)hydroxymethyl]-4-cyclooctyl-2,4-dihydro-[1,2,4]triazole-3-thione;
5-[(2-chlorophenyl)hydroxymethyl]-4-(2,2-dimethoxyethyl)-2,4-dihydro-[1,2,4]triazole-3-thione;
5-[(2-chlorophenyl)hydroxymethyl]-4-(2-methylbutyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro-[1,2,4]triazole-3-thione;
4-phenyl-5-(pyrrol-2-yl)-2,4-dihydro-[1,2,4]triazole-3-thione;
5-(2-chlorobenzyl)-4-(3-hydroxymethylphenyl)-2,4-dihydro-(1,2,4]triazole-3-thione;
5-(4-chlorobenzyl)-4-phenyl-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
4-phenyl-5-pyridin-3-yl-methyl-2,4-dihydro-[1,2,4]triazole-3-thione;
5-(3-chlorobenzyl)-4-(2-methoxyphenyl)-2,4-dihydro-[1,2,4]triazole-3-thione;
5-(3-methoxybenzyl)-4-phenyl-2,4-dihydro-[1,2,4]triazole-3-thione;
5-(2-bromo-5-methylbenzyl)-4-phenyl-2,4-dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(furan-2-ylmethyl)-4-phenyl-2,4-dihydro-[1,2,4]triazole-3-thione;
5-(3-methylbenzyl)-4-phenyl-2,4-dihydro-[1,2,4]triazole-3-thione;
5-(2-chlorobenzyl)-4-(4-methylphenyl)-2,4-dihydro-[1,2,4]triazole-3-thione;
5-(2-hydroxy-1-phenylethyl)-4-phenyl-2,4-dihydro-[1,2,4]triazole-3-thione;
5-(3,5-dimethylbenzyl)-4-phenyl-2,4-dihydro-[1,2,4]triazole-3-thione;
5-(2,3-dichlorobenzyl)-4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(3-trifluoromethylbenzyl)-4-phenyl-2,4-dihydro-[1,2,4]triazole-3-thione;
5-phenoxy4-phenyl-2,4-dihydro-[1,2,4]triazole-3-thione;
5-(2-methylbenzyl)-4-phenyl-2,4-dihydro-[1,2,4]triazole-3-thione;
5-[(2-chlorophenyl)hydroxymethyl]-4-cyclohexyl-2,4-dihydro-[1,2,4]triazole-3-thione;
5-[(2-chlorophenyl)hydroxymethyl]-4-phenyl-2,4-dihydro-[1,2,4]triazole-3-thione;
5-[(2-chlorophenyl)hydroxymethyl]-4-(3-methoxypropyl)-2,4-dihydro-[1,2,4]triazole-3-thione;
5-(2-chlorobenzyl)-4-(2-piperidin-1-yl-ethyl)-2,4-dihydro-[1,2,4]triazole-3-thione;
4-butyl-5-(2-chlorobenzyl)-2,4-dihydro-[1,2,4]triazole-3-thione;
5-(2-chlorobenzyl)-4-(3-morpholin-4-yl-propyl)-2,4-dihydro-[1,2,4]triazole-3-thione;
5-(2-chlorobenzyl)-4-(tetrahydrofuran-2-yl-methyl)-2,4-dihydro-[1,2,4]triazole-3-thione;
5-(2-chlorobenzyl)-4-(4-chlorophenyl)-2,4-dihydro-[1,2,4]triazole-3-thione;
5-(1H-indol-3-ylmethyl)-4-(2-methoxyphenyl)-2,4-dihydro-[1,2,4]triazole-3-thione;
5-(1H-indol-3-ylmethyl)-4-(2-methylphenyl)-2,4-dihydro-[1,2,4]triazole-3-thione;
5-cyclopentylmethyl-4-phenyl-2,4-dihydro-[1,2,4]triazole-3-thione;
5-(2-chlorobenzyl)-4-(2-chlorophenyl)-2,4-dihydro-[1,2,4]triazole-3-thione;
5-[(2-chlorophenyl)hydroxymethyl]-4-(4-nitrophenyl)-2,4-dihydro-[1,2,4]triazole-3-thione;
5-(2,3-dichlorophenoxymethyl)-4-phenyl-2,4-dihydro-[1,2,4]triazole-3-thione;
5-(4-chloro-2-methylphenoxymethyl)-4-phenyl-2,4-dihydro-[1,2,4]triazole-3-thione;
5-(2-chlorobenzyl)4-phenyl-2,4-dihydro-[1,2,4]triazole-3-thione;
5-(4-bromophenoxymethyl)-4-phenyl-2,4-dihydro-[1,2,4]triazole-3-thione;
5-(1H-indol-3-ylmethyl)-4-phenyl-2,4-dihydro-[1,2,4]triazole-3-thione;
5-(3-chlorobenzyl)-4-phenyl-2,4-dihydro-[1,2,4]triazole-3-thione;
5-(6-bromonaphthalen-2-yloxylmethyl)-4-phenyl-2,4-dihydro-[1,2,4]triazole-3-thione;
5-(4-methoxyphenoxymethyl)-4-phenyl-2,4-dihydro-[1,2,4]triazole-3-thione;
5-(3,4-dimethoxybenzyl)-4-phenyl-2,4-dihydro-[1,2,4]triazole-3-thione;
5-(3-methoxyphenyl)-4-phenyl-2,4-dihydro-[1,2,4]triazole-3-thione;
5-(3-dimethylaminophenoxymethyl)-4-phenyl-2,4-dihydro-[1,2,4]triazole-3-thione;
4-phenyl-5-thiophen-2-yl-2,4-dihydro-[1,2,4]triazole-3-thione;
5-(4-hydroxyphenyl)-4-phenyl-2,4-dihydro-[1,2,4]triazole-3-thione;
5-(4-carboxyphenoxy)methyl-4-(4-chlorophenyl)-2,4-dihydro-[1,2,4]triazole-3-thione;
5-(hydroxyphenylmethyl)-4-phenyl-2,4-dihydro-[1,2,4]triazole-3-thione;
5-benzyl-4-phenyl-2,4-dihydro-[1,2,4]triazol-3-thione;
4-(3-chlorophenyl)-5-(5-methyl-2-nitrophenyl)-2,4-dihydro-[1,2,4]triazole-3-thione;
4-phenyl-5-(4-trifluoromethoxyphenoxymethyl)-2,4-dihydro-[1,2,4]triazole-3-thione;
4-phenyl-5-(4-trifluoromethylsulfanyl-phenoxymethyl)-2,4-dihydro-[1,2,4]triazole-3-thione;
5-(4-cyclohexylphenoxymethyl)-4-phenyl-2,4-dihydro-[1,2,4]triazole-3-thione;
4-phenyl-5-phenylamino-2,4-dihydro-[1,2,4]triazole-3-thione;
4-phenyl-5-thiophen-2-ylmethyl-2,4-dihydro-[1,2,4]triazole-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(3-butoxybenzyl)-4-phenyl-2,4-dihydro-[1,2,4]triazole-3-thione;
5-naphthalen-1-ylmethyl-4-phenyl-2,4-dihydro-[1,2,4]triazole-3-thione;
5-(2-chloro-5-methoxybenzyl)-4-phenyl-2,4-dihydro-[1,2,4]triazole-3-thione;
5-(3-chlorobenzyl)-4-o-tolyl-2,4-dihydro-[1,2,4]triazole-3-thione;
5-(2chloro-6-fluoro-3-methylbenzyl)4-phenyl-2,4-dihydro-[1,2,4]triazole-3-thione;
5-(biphenyl-2-ylmethyl)-4-phenyl-2,4-dihydro-[1,2,4]triazole-3-thione;
5-(3-oxo-indan-1-yl)-4-phenyl-2,4-dihydro-[1,2,4]triazole-3-thione;
5-(4-chlorophenoxy)methyl-4-phenyl-2,4-dihydro-[1,2,4]triazole-3-thione;
5-(4-acetylphenoxy)methyl-4-phenyl-2,4-dihydro-[1,2,4]triazole-3-thione;
5-(3-methoxyphenoxy)methyl-4-phenyl-2,4-dihydro-[1,2,4]triazole-3-thione;
5-(2-methoxyphenoxy)methyl-4-phenyl-2,4-dihydro-[1,2,4]triazole-3-thione;
5-phenoxymethyl-4-phenyl-2,4-dihydro-[1,2,4]triazole-3-thione;
5-(4-butoxyphenoxy)methyl-4-phenyl-2,4-dihydro-[1,2,4]triazole-3-thione;
5-(2-chlorophenoxy)methyl-4-phenyl-2,4-dihydro-[1,2,4]triazole-3-thione;
5-(3-chlorophenoxy)methyl-4-phenyl-2,4-dihydro-[1,2,4triazole-3-thione;
5-(2-methylcarbamoylphenoxy)methyl-4-phenyl-2,4-dihydro-[1,2,4]triazole-3-thione;
5-(3-butoxy-phenoxymethyl)-4-phenyl-2,4-dihydro-[1,2,4]triazole-3-thione;
5-isochroman-1-yl4-phenyl-2,4-dihydro-[1,2,4]triazole-3-thione;
5-{3-[(methylamino)carbonyl]benzyl}-4-phenyl-2,4-dihydro-[1,2,4]triazole-3-thione;
5-naphthalen-2-ylmethyl-4-phenyl-2,4-dihydro-[1,2,4]triazole-3-thione;
4-phenyl-5-(pyridin-2-ylmethyl)-2,4-dihydro-[1,2,4]triazole-3-thione;
5-(2,3-dimethoxybenzyl)-4-phenyl-2,4-dihydro-[1,2,4]triazole-3-thione;
4-phenyl-5-(2,3,4-trimethoxybenzyl)-2,4-dihydro-[1,2,4]-triazole-3-thione;
5-[(2,5-dimethyl-1,3-thiazol-4-yl)methyl]-4-phenyl-2,4-dihydro-[1,2,4]triazole-3-thione;
4-phenyl-5-(2-phenylethyl)-2,4-dihydro-[1,2,4]triazole-3-thione;
5-[(2-butoxyphenoxy)methyl]-4-phenyl-2,4-dihydro-[1,2,4]triazole-3-thione;
4-phenyl-5-(tetrahydrofuran-2-ylmethyl)-2,4-dihydro-[1,2,4]triazole-3-thione;
4-[4-(2,6-dimethyl-morpholin-4-yl)-phenyl]-5-(diphenylmethyl)-2,4-dihydro-[1,2,4]triazole-3-thione;
5-benzyl-4-(2-furylmethyl)-2,4-dihydro-[1,2,4]triazole-3-thione;
5-benzyl-4-(3,5-dimethyl-isoxazol-4-yl)-2,4dihydro-[1,2,4]triazole-3-thione;
5-benzyl-4-(5-methyl-3-phenyl-isoxazol-4-yl)-2,4-dihydro-[1,2,4]triazole-3-thione;
4-(2,1,3-benzothiadiazol-4-yl)-5-benzyl-2,4-dihydro-[1,2,4]triazole-3-thione;
5-benzyl-4-pyridin-2-yl-2,4-dihydro-[1,2,4]triazole-3-thione;
5-benzyl-4-(2-cyanophenyl)-2,4-dihydro-[1,2,4]triazole-3-thione;
5-benzyl-4-(2-thienyl)-2,4-dihydro-[1,2,4]triazole-3-thione;
5-benzyl-4-[2-(2-thienyl)ethyl]-2,4-dihydro-[1,2,4]triazole-3-thione;
5-(2-chlorobenzyl)-4-(3-diethylaminopropyl)-2,4-dihydro-[1,2,4]triazole-3-thione; or a pharmaceutically acceptable salt thereof.

In one embodiment, the invention concerns the use of any one or more of the following compounds of formula (I):
5-(4-aminobenzyl)-4-[3,5-di(trifluoromethyl)phenyl]-2,4-dihydro-[1,2,4]triazole-3-thione;
5-(4-chlorobenzyl)-4-(4-methylphenyl)-2,4-dihydro-[1,2,4]triazole-3-thione;
5-(4-hydroxybenzyl)-4-[3-(methylthio)phenyl]-2,4dihydro-[1,2,4]triazole-3-thione;
5-(2,5-dimethoxybenzyl)4-[3-(methylthio)phenyl]-2,4dihydro-[1,2,4]triazole-3-thione;
5-(4-hydroxybenzyl)-4-(4-iodophenyl)-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(4-hydroxybenzyl)-4-[4-(piperidinosulfonyl)phenyl]-2,4-dihydro-[1,2,4]triazole-3-thione;
5-(2,5-dimethoxybenzyl])-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
5-(4-hydroxybenzyl)-4-(2,4,6-trichlorophenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
5-(2-bromobenzyl)-4-(4-sulfamoylphenyl)-2,4-dihydro-[1,2,4]triazole-3-thione;
5-(4-hydroxybenzyl)-4-(2,6-dibromo-4-methylphenyl)-2,4-dihydro-[1,2,4]triazole-3-thione;
5-(4-hydroxybenzyl)-4-(3,4,5-trimethoxyphenyl)-2,4dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(2-chlorobenzyl)-4-(2-ethoxyphenyl)-2,4-dihydro-[1,2,4]triazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4-dihydro-[1,2,4]triazole-3-thione;
5-(2-chlorobenzyl)-4-(3-methylphenyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro-[1,2,4]triazole-3-thione;
5-(2-chlorobenzyl)-4-(3-hydroxymethylphenyl)-2,4-dihydro-[1,2,4]triazole-3-thione;
5-(4-chlorobenzyl)-4-phenyl-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
5-(3-chlorobenzyl)-4-(2-methoxyphenyl)-2,4-dihydro-[1,2,4]triazole-3-thione;
5-(3-methoxybenzyl)-4-phenyl-2,4-dihydro-[1,2,4]triazole-3-thione;
5-(2-bromo-5-methylbenzyl)-4-phenyl-2,4-dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(3-methylbenzyl)-4-phenyl-2,4-dihydro-[1,2,4]triazole-3-thione;
5-(2-chlorobenzyl)-4-(4-methylphenyl)-2,4-dihydro-[1,2,4]triazole-3-thione;
5-(3,5-dimethylbenzyl)-4-phenyl-2,4-dihydro-[1,2,4]triazole-3-thione;
5-(2,3-dichlorobenzyl)-4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(3-trifluoromethylbenzyl)-4-phenyl-2,4-dihydro-[1,2,4]triazole-3-thione;
5-(2-methylbenzyl)-4-phenyl-2,4-dihydro-[1,2,4]triazole-3-thione;
5-(2-chlorobenzyl)-4-(4-chlorophenyl)-2,4-dihydro-[1,2,4]triazole-3-thione;
5-(2-chlorobenzyl)-4-(2-chlorophenyl)-2,4-dihydro-[1,2,4]triazole-3-thione;
5-(2-chlorobenzyl)-4-phenyl-2,4-dihydro-[1,2,4]triazole-3-thione;
5-(3-chlorobenzyl)-4-phenyl-2,4-dihydro-[1,2,4]triazole-3-thione;
5-(3,4dimethoxybenzyl)-4-phenyl-2,4-dihydro-[1,2,4]triazole-3-thione;
5-(hydroxyphenylmethyl)-4-phenyl-2,4-dihydro-[1,2,4]triazole-3-thione;
5-benzylphenyl-2,4-dihydro-[1,2,4]triazol-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(3-butoxybenzyl)-4-phenyl-2,4-dihydro-[1,2,4]triazole-3-thione;
5-(2-chloro-5-methoxybenzyl)-4-phenyl-2,4-dihydro-[1,2,4]triazole-3-thione;
5-(3-chlorobenzyl)-4-o-tolyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(biphenyl-2-ylmethyl)-4-phenyl-2,4-dihydro-[1,2,4]triazole-3-thione;
5-{3-[(methylamino)carbonyl]benzyl }-4-phenyl-2,4-dihydro-[1,2,4]triazole-3-thione;
5-(2,3-dimethoxybenzyl)-4-phenyl-2,4-dihydro-[1,2,4]triazole-3-thione;
4-phenyl-5-(2,3,4-trimethoxybenzyl)-2,4-dihydro-[1,2,4]-triazole-3-thione;
5-benzyl-4-(2-cyanophenyl)-2,4-dihydro-[1,2,4]triazole-3-thione;
   or a pharmaceutically acceptable salt thereof.

In one embodiment, the invention concerns the use of any one or more of the following compounds of formula (I):
5-(4-aminobenzyl)-4-[3,5-di(trifluoromethyl)phenyl]-2,4-dihydro-[1,2,4]triazole-3-thione;
5-(4-chlorobenzyl)-4-(4-methylphenyl)-2,4-dihydro-[1,2,4]triazole-3-thione;
5-(4-hydroxybenzyl)-4-[3-(methylthio)phenyl]-2,4-dihydro-[1,2,4]triazole-3-thione;
5-(4-hydroxybenzyl)-4-(4-iodophenyl)-2,4-dihydro-[1,2,4]triazole-3-thione;
5-(4-hydroxybenzyl)-4-[4-(piperidinosulfonyl)phenyl]-2,4-dihydro-[1,2,4]triazole-3-thione;
5-(4-hydroxybenzyl)-4-(2,4,6-trichlorophenyl)-2,4-dihydro-[1,2,4]triazole-3-thione;
5-(4-hydroxybenzyl)-4-(2,6-dibromo-4-methylphenyl)-2,4-dihydro-[1,2,4]triazole-3-thione;
5-(4-hydroxybenzyl)-4-(3,4,5-trimethoxyphenyl)-2,4-dihydro-[1,2,4]triazole-3-thione;
5-(4-chlorobenzyl)-4-phenyl-2,4-dihydro-[1,2,4]triazole-3-thione;
5-(3-chlorobenzyl)-4-(2-methoxyphenyl)-2,4-dihydro-[1,2,4]triazole-3-thione;
5-(3-methoxybenzyl)-4-phenyl-2,4-dihydro-[1,2,4]triazole-3-thione;
5-(3-methylbenzyl)-4-phenyl-2,4-dihydro-[1,2,4]triazole-3-thione;
5-(3,5-dimethylbenzyl)-4-phenyl-2,4-dihydro-[1,2,4]triazole-3-thione;
5-(3-trifluoromethylbenzyl)-4-phenyl-2,4-dihydro-[1,2,4]triazole-3-thione;
5-(3-chlorobenzyl)-4-phenyl-2,4-dihydro-[1,2,4]triazole-3-thione;
5-(3,4-dimethoxybenzyl)-4-phenyl-2,4-dihydro-[1,2,4]triazole-3-thione;
5-(hydroxyphenylmethyl)-4-phenyl-2,4-dihydro-[1,2,4]triazole-3-thione;
5-benzyl-4-phenyl-2,4-dihydro-[1,2,4]triazol-3-thione;
5-(3-butoxybenzyl)-4-phenyl-2,4-dihydro-[1,2,4]triazole-3-thione;
5-(3-chlorobenzyl)-4-o-tolyl-2,4-dihydro-[1,2,4]triazole-3-thione;
5-(biphenyl-2-ylmethyl)-4-phenyl-2,4-dihydro-[1,2,4]triazole-3-thione;
5-{3-[(methylamino)carbonyl]benzyl}-4-phenyl-2,4-dihydro-[1,2,4]triazole-3-thione;
5-benzyl-4-(2-cyanophenyl)-2,4-dihydro-[1,2,4]triazole-3-thione;
   or a pharmaceutically acceptable salt thereof.

In one embodiment, the invention concerns the use of any one or more of the following compounds of formula (I):
5-(4-methoxyphenoxymethyl)-4-(2-tetrahydrofuran-2-yl-methyl)-2,4-dihydro-[1,2,4]triazole-3-thione;
5-(4-methoxyphenoxymethyl)-4-(3-methoxypropyl)-2,4-dihydro-[1,2,4]triazole-3-thione;
5-(4-methoxyphenoxymethyl)-4-(2-phenylethyl)-2,4-dihydro-[1,2,4]triazole-3-thione;
5-(4-methoxyphenoxymethyl)-4-(3-morpholin-4-yl-propyl)-2,4-dihydro-[1,2,4]triazole-3-thione;
5-(4-methoxyphenoxymethyl)-4-cyclopropyl-2,4-dihydro-[1,2,4]triazole-3-thione;
5-(2,3-dichlorophenoxymethyl)-4-phenyl-2,4-dihydro-[1,2,4]triazole-3-thione;
5-(4-chloro-2-methylphenoxymethyl)-4-phenyl-2,4-dihydro-[1,2,4]triazole-3-thione;
5-(4-bromophenoxymethyl)-4-phenyl-2,4-dihydro[1,2,4]triazole-3-thione;
5-(6-bromonaphthalen-2-yloxylmethyl)-4-phenyl-2,4-dihydro-[1,2,4]triazole-3-thione;
5-(4-methoxyphenoxymethyl)-4-phenyl-2,4-dihydro-[1,2,4]triazole-3-thione;
5-(3-dimethylaminophenoxymethyl)-4-phenyl-2,4-dihydro-[1,2,4]triazole-3-thione;
5-(4-carboxyphenoxy)methyl-4-(4-chlorophenyl)-2,4-dihydro-[1,2,4]triazole-3-thione;
4-phenyl-5-(4-trifluoromethoxyphenoxymethyl)-2,4-dihydro-[1,2,4]triazole-3-thione;
4-phenyl-5-(4-trifluoromethylsulfanyl-phenoxymethyl)-2,4-dihydro-[1,2,4]triazole-3-thione;
5-(4-cyclohexylphenoxymethyl)-4-phenyl-2,4-dihydro-[1,2,4]triazole-3-thione;
5-(4-chlorophenoxy)methyl-4-phenyl-2,4-dihydro-[1,2,4]triazole-3-thione;
5-(4-acetylphenoxy)methyl-4-phenyl-2,4-dihydro-[1,2,4]triazole-3-thione;
5-(3-methoxyphenoxy)methyl-4-phenyl-2,4-dihydro-[1,2,4]triazole-3-thione;
5-(2-methoxyphenoxy)methyl-4-phenyl-2,4-dihydro-[1,2,4]triazole-3-thione;
5-phenoxymethyl-phenyl-2,4-dihydro-[1,2,4]triazole-3-thione;
5-(4-butoxyphenoxy)methyl-4-phenyl-2,4-dihydro-[1,2,4]triazole-3-thione;
5-(2-chlorophenoxy)methyl-4-phenyl-2,4-dihydro-[1,2,4]triazole-3-thione;
5-(3-chlorophenoxy)methyl-4-phenyl-2,4-dihydro-[1,2,4]triazole-3-thione;
5-(2-methylcarbamoylphenoxy)methyl-4-phenyl-2,4-dihydro-[1,2,4]triazole-3-thione;
5-(3-butoxy-phenoxymethyl)-4-phenyl-2,4-dihydro-[1,2,4]triazole-3-thione;
5-[(2-butoxyphenoxy)methyl]-4phenyl-2,4-dihydro-[1,2,4]triazole-3-thione;
   or a pharmaceutically acceptable salt thereof.

Unless otherwise indicated, the term “C1 to 6 alkyl” referred to herein denotes a straight or branched chain alkyl group having from 1 to 6 carbon atoms. Examples of such groups include methyl, ethyl, 1-propyl, n-butyl, iso-butyl, tert-butyl, pentyl and hexyl. The term “C1 to 2 alkyl” is to be interpreted analogously.

Unless otherwise indicated, the term “C3 to 8 cycloalkyl” referred to herein denotes a cyclic alkyl group having from 3 to 8 carbon atoms. Examples of such groups include cyclopropyl, cyclopentyl and cyclohexyl. The term “C3 to 6 cycloalkyl” is to be interpreted analogously. The term “C3 to 6 cycloalkyl; said cycloalkyl group optionally including an O atom and optionally being benzo fused” is to be interpreted analogously. Examples of such groups include tetrahydrofuran, oxane, indan, tetrahydronaphthalene, chroman and isochroman,

Unless otherwise indicated, the term “C1 to 6 alkoxy” referred to herein denotes a straight or branched chain alkoxy group having from 1 to 6 carbon atoms. Examples of such groups include methoxy, ethoxy, 1-propoxy, 2-propoxy and tert-butoxy.

The term “C1 to 2 alkoxy” is to be interpreted analogously.

Unless otherwise indicated, the term “C1 to 6 alkylthio” referred to herein denotes a straight or branched chain alkyl group having from 1 to 6 carbon atoms that is bonded to the molecule via a sulphur atom. Examples of such groups include methylthio, ethylthio and propylthio.

Unless otherwise indicated, the term “C2 to 6 alkanoyl” referred to herein denotes a straight or branched chain alkyl group having from 1 to 5 carbon atoms bonded through a carbonyl group. Examples of such groups include acetyl, propionyl and pivaloyl.

Unless otherwise indicated, the term “halogen” referred to herein denotes fluoro, chloro, bromo and iodo.

Examples of an alkyl or alkoxy group optionally further substituted by one or more fluoro atoms include CH₂F, CHF₂, CF₃, CF₃CF₂, CF₃CH₂, CH₂FCH₂, CH₃CF₂, CF₃CH₂CH₂, OCF₃ and OCH₂CF₃.

Examples of a 5- or 6-membered heteroaromatic ring containing one or two heteroatoms independently selected from O, S and N include furan, thiophene, imidazole, thiazole, isoxazole, pyridine and pyrimidine.

Examples of a 5- or 6-membered saturated heterocyclic ring containing one or two heteroatoms independently selected from 0, N and S include tetrahydrofuran, pyrrolidine, piperidine, morpholine, thiomorpholine and piperazine.

Examples of a monocyclic or bicyclic heteroaromatic ring system containing one to three heteroatoms independently selected from O, N and S include furan, thiophene, imidazole, thiazole, isoxazole, pyridine, pyrimidine, indole, isoquinoline, benzofuran and benzothiadiazole.

Examples of a saturated 5- or 6-membered azacyclic ring optionally including one further heteroatom selected from O, S and N include pyrrolidine, morpholine, piperazine and piperidine.

Certain compounds of formula (I) are novel. A further aspect of the invention thus provides the following novel compounds of formula (I):
5-(4-aminobenzyl)-4-[3,5-di(trifluoromethyl)phenyl]-2,4-dihydro-[1,2,4]triazole-3-thione;
5-(4-chlorobenzyl)-4-(4-methylphenyl)-2,4-dihydro-[1,2,4]triazole-3-thione;
5-isobutyl-4-phenyl-2,4-dihydro-[1,2,4]triazole-3-thione;
5-(4-hydroxybenzyl)-4-[3-(methylthio)phenyl]-2,4-dihydro-[1,2,4]triazole-3-thione;
5-(2,5-dimethoxybenzyl)4-[3-(methylthio)phenyl]-2,4-dihydro-[1,2,4]triazole-3-thione;
5-(4-hydroxybenzyl)-4-(4-iodophenyl)-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(4-hydroxybenzyl)-4-[4-(piperidinosulfonyl)phenyl]-2,4-dihydro-[1,2,4]triazole-3-thione;
5-(2,5-dimethoxybenzyl)-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
5-(4-hydroxybenzyl)-4-(2,4,6-trichlorophenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
4-(4-carboxyphenyl)-5-(diphenylmethyl)-2,4-dihydro-[1,2,4]triazole-3-thione;
5-(2-bromobenzyl)-4-(4-sulfamoylphenyl)-2,4-dihydro-[1,2,4]triazole-3-thione;
5-(4-hydroxybenzyl)-4-(naphthalen-1-yl)-2,4-dihydro-[1,2,4]triazole-3-thione;
5-(4-hydroxybenzyl)-4-(2,6-dibromo-4-methylphenyl)-2,4-dihydro-[1,2,4]triazole-3-thione;
5-(4-hydroxybenzyl)-4-(3,4,5-trimethoxyphenyl)-2,4-dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(4-methoxyphenoxymethyl)-4-(2-tetrahydrofuran-2-yl-methyl)-2,4-dihydro-[1,2,4]triazole-3-thione;
5-(4-methoxyphenoxymethyl)-4-(3-methoxypropyl)-2,4-dihydro-[1,2,4]triazole-3-thione;
5-(4-methoxyphenoxymethyl)-4-(2-phenylethyl)-2,4-dihydro-[1,2,4]triazole-3-thione;
5-(4-methoxyphenoxymethyl)-4-(3-morpholin-4-yl-propyl)-2,4-dihydro-[1,2,4]triazole-3-thione;
4-butyl-5-[(4-methoxyphenylamino)-methyl]-2,4-dihydro-[1,2,4]triazole-3-thione;
5-[(4methoxyphenylamino)-methyl]-4-(3-methoxypropyl)-2,4-dihydro-[1,2,4]triazole-3-thione;
4-hexyl-5-[(4-methoxyphenylamino)methyl]-2,4-dihydro-[1,2,4]triazole-3-thione;
5-(2-chlorobenzyl)-4-(2-ethoxyphenyl)-2,4-dihydro-[1,2,4]triazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-pyridin-3-yl-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(4-methoxyphenoxymethyl)-4-cyclopropyl-2,4-dihydro-[1,2,4]triazole-3-thione;
4-(2,2-dimethoxyethyl)-5-[(4-methoxyphenylamino)methyl]-2,4-dihydro-[1,2,4]triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4dihydro-[1,2,4]triazole-3-thione;
5-(2-chlorobenzyl)-4-(3-methylphenyl)-2,4-dihydro-[1,2,4]triazole-3-thione;
5-[(2-chlorophenyl)hydroxymethyl]-4-isobutyl-2,4-dihydro-[1,2,4]triazole-3-thione;
5-[(2-chloro-phenyl)hydroxymethyl]-4-cyclooctyl-2,4-dihydro-[1,2,4]triazole-3-thione;
5-[(2-chlorophenyl)hydroxymethyl]-4-(2,2-dimethoxyethyl)-2,4-dihydro-[1,2,4]triazole-3-thione;
5-[(2-chlorophenyl)hydroxymethyl]-4-(2-methylbutyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro-[1,2,4]triazole-3-thione;
4-phenyl-5-(pyrrol-2-yl)-2,4-dihydro-[1,2,4]triazole-3-thione;
5-(2-chlorobenzyl)-4-(3-hydroxymethylphenyl)-2,4-dihydro-[1,2,4]triazole-3-thione;
5-(4-chlorobenzyl)-4-phenyl-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
4-phenyl-5-pyridin-3-yl-methyl-2,4-dihydro-[1,2,4]triazole-3-thione;
5-(3-chlorobenzyl)-4-(2-methoxyphenyl)-2,4-dihydro-[1,2,4]triazole-3-thione;
5-(3-methoxybenzyl)-4-phenyl-2,4-dihydro-[1,2,4]triazole-3-thione;
5-(2-bromo-5-methylbenzyl)4-phenyl-2,4dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(furan-2-ylmethyl)-4-phenyl-2,4-dihydro-[1,2,4]triazole-3-thione;
5-(3-methylbenzyl)-4-phenyl-2,4-dihydro-[1,2,4]triazole-3-thione;
5-(2-chlorobenzyl)-4-(4-methylphenyl)-2,4-dihydro-[1,2,4]triazole-3-thione;
5-(2-hydroxy-1-phenylethyl)-4-phenyl-2,4-dihydro-[1,2,4]triazole-3-thione;
5-(3,5-dimethylbenzyl)-4-phenyl-2,4-dihydro-[1,2,4]triazole-3-thione;
5-(2,3-dichlorobenzyl)4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(3-trifluoromethylbenzyl)-4-phenyl-2,4-dihydro-[1,2,4]triazole-3-thione;
5-phenoxy-4-phenyl-2,4-dihydro-[1,2,4]triazole-3-thione;
5-(2-methylbenzyl)-4-phenyl-2,4-dihydro-[1,2,4]triazole-3-thione;
5-[(2-chlorophenyl)hydroxymethyl]-4-cyclohexyl-2,4-dihydro-[1,2,4]triazole-3-thione;
5-[(2-chlorophenyl)hydroxymethyl]-4-phenyl-2,4-dihydro-[1,2,4]triazole-3-thione;
5-[(2-chlorophenyl)hydroxymethyl]-4-(3-methoxypropyl)-2,4-dihydro-[1,2,4]triazole-3-thione;
5-(2-chlorobenzyl)-4-(2-piperidin-1-yl-ethyl)-2,4-dihydro-[1,2,4]triazole-3-thione;
4-butyl-5-(2-chlorobenzyl)-2,4-dihydro-[1,2,4]triazole-3-thione;
5-(2-chlorobenzyl)-4-(3-morpholin-4-yl-propyl)-2,4-dihydro-[1,2,4]triazole-3-thione;
5-(2-chlorobenzyl)-4-(tetrahydrofuran-2-yl-methyl)-2,4-dihydro-[1,2,4]triazole-3-thione;
5-(2-chlorobenzyl)-4-(4-chlorophenyl)-2,4-dihydro-[1,2,4]triazole-3-thione;
5-(1H-indol-3-ylmethyl)-4-(2-methoxyphenyl)-2,4-dihydro-[1,2,4]triazole-3-thione;
5-(1H-indol-3-ylmethyl)-4-(2-methylphenyl)-2,4-dihydro-[1,2,4]triazole-3-thione;
5-cyclopentylmethyl-4-phenyl-2,4-dihydro-[1,2,4]triazole-3-thione;
5-(2-chlorobenzyl)4-(2-chlorophenyl)-2,4-dihydro-[1,2,4]triazole-3-thione;
5-[(2-chlorophenyl)hydroxymethyl]-4-(4-nitrophenyl)-2,4-dihydro-[1,2,4]triazole-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(3-butoxybenzyl)-4-phenyl-2,4-dihydro-[1,2,4]triazole-3-thione;
5-(2-chloro-5-methoxybenzyl)-4-phenyl-2,4-dihydro-[1,2,4]triazole-3-thione;
5-(3-chlorobenzyl)-4-o-tolyl-2,4-dihydro-[1,2,4]triazole-3-thione;
5-(6-chloro-2-fluoro-3-methylbenzyl)-4-phenyl-2,4-dihydro-[1,2,4]triazole-3-thione;
5-(biphenyl-2-ylmethyl)-4-phenyl-2,4-dihydro-(1,2,4]triazole-3-thione;
5-(3-oxo-indan-1-yl)-4-phenyl-2,4-dihydro-[1,2,4]triazole-3-thione;
5-(4-acetylphenoxy)methyl-4-phenyl-2,4-dihydro-[1,2,4]triazole-3-thione;
5-(3-methoxyphenoxy)methyl-4-phenyl-2,4-dihydro-[1,2,4]triazole-3-thione;
5-(4-butoxyphenoxy)methyl-4-phenyl-2,4dihydro-[1,2,4]triazole-3-thione;
5-(3-chlorophenoxy)methyl-4-phenyl-2,4-dihydro-[1,2,4]triazole-3-thione;
5-(2-methylcarbamoylphenoxy)methyl-4-phenyl-2,4-dihydro-[1,2,4]triazole-3-thione;
5-(3-butoxy-phenoxymethyl)-4-phenyl-2,4-dihydro-[1,2,4]triazole-3-thione;
5-isochroman-1-yl-4-phenyl-2,4-dihydro-[1,2,4]triazole-3-thione;
5-{3-[(methylamino)carbonyl]benzyl}-4-phenyl-2,4dihydro-[1,2,4]triazole-3-thione;
4-phenyl-5-(pyridin-2-ylmethyl)-2,4-dihydro-[1,2,4]triazole-3-thione;
5-(2,3-dimethoxybenzyl)-4-phenyl-2,4-dihydro-[1,2,4]triazole-3-thione;
4-phenyl-5-(2,3,4-trimethoxybenzyl)-2,4-dihydro-[1,2,4]-triazole-3-thione;
5-[(2,5-dimethyl-1,3-thiazol-4-yl)methyl]-4-phenyl-2,4-dihydro-[1,2,4]triazole-3thione
5-[(2-butoxyphenoxy)methyl]-4-phenyl-2,4-dihydro-[1,2,4]triazole-3-thione;
4-phenyl-5-(tetrahydrofuran-2-ylmethyl)-2,4-dihydro-[1,2,4]triazole-3-thione;
4-[4-(2,6-dimethyl-moipholin-4-yl)-phenyl]-5-(diphenylmethyl)-2,4-dihydro-[1,2,4]triazole-3-thione;
5-benzyl-4-(2-furylmethyl)-2,4-dihydro-[1,2,4]triazole-3-thione;
5-benzyl-4-(3,5-dimethyl-isoxazol-4-yl)-2,4-dihydro-[1,2,4]triazole-3-thione;
5-benzyl-4-(5-methyl-3-phenyl-isoxazol-4-yl)-2,4dihydro-[1,2,4]triazole-3-thione;
4-(2,1,3-benzothiadiazol-4-yl)-5-benzyl-2,4-dihydro-[1,2,4]triazole-3-thione;
5-benzyl-4-(2-cyanophenyl)-2,4-dihydro-[1,2,4]triazole-3-thione;
5-benzyl-4-(2-thienyl)-2,4-dihydro-[1,2,4]triazole-3-thione;
5-benzyl4-[2-(2-thienyl)ethyl]-2,4-dihydro-[1,2,4]triazole-3-thione;
5-(2-chlorobenzyl)-4-(3-diethylaminopropyl)-2,4-dihydro-[1,2,4]triazole-3-thione;
   and pharmaceutically acceptable salts thereof.

A further aspect of the invention concerns the novel compounds of formula (I) for use as a medicament.

In a further aspect, the present invention provides novel compounds of formula (Ia) 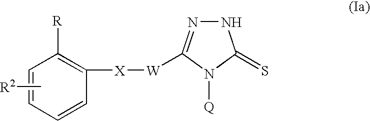
wherein:
Q represents phenyl optionally substituted by one to three substituents independently selected from halogen, CN, C1 to 6 alkyl, C1 to 6 alkoxy, C1 to 6 alkylthio, CO₂R⁶, COR⁷, CH₂OH, Ph, NO₂, NR⁸R⁹ and SO₂NR¹⁰R¹¹; said alkyl or alkoxy group being optionally further substituted by one or more fluoro atoms;
W represents CH₂;
X represents a bond;
R represents halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
R² represents H or one or more substituents independently selected from halogen, OH, C1 to 6 alkyl, C3 to 6 cycloalkyl, C1 to 6 alkoxy, C1 to 6 alkylthio, CO₂H, C2 to 6 alkanoyl,
Ph, NO₂, C(O)NR¹²R¹³ or NR⁴R⁵; said alkyl, cycloalkyl, alkoxy and alkylthio groups being optionally further substituted by one or more fluoro atoms;
each R⁴, R⁵, R⁶, R⁷, R¹² and R¹³ independently represents H or C1 to 6 alkyl;
each R⁸, R⁹, R¹⁰ and R¹¹ independently represents H or C1 to 6 alkyl; or the group NR⁸R⁹ or NR¹⁰R¹¹ together represents a saturated 5- or 6-membered azacyclic ring optionally including one further heteroatom selected from O, S and N, and optionally being substituted by one or more C1 to 6 alkyl groups;
   and pharmaceutically acceptable salts thereof; with the proviso that the following compounds are excluded:
5-[(2-chlorophenyl)methyl]-2,4-dihydro-4-phenyl-3H-1,2,4-triazole-3-thione;
5-[(2-chloro-6-fluorophenyl)methyl]-2,4-dihydro-4-phenyl-3H-1,2,4-triazole-3-thione.

Particular compounds of formula (Ia) include:
5-(2,5-dimethoxybenzyl)-4-[3-(methylthio)phenyl]-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(2,5-dimethoxybenzyl)-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
5-(2-bromobenzyl)-4-(4-sulfamoylphenyl)-2,4-dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(2-chlorobenzyl)4(2-ethoxyphenyl)-2,4-dihydro-[1,2,4]tiazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4-dihydro-[1,2,4]triazole-3-thione;
5-(2-chlorobenzyl)-4-(3-methylphenyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro-[1,2,4]triazole-3-thione;
5-(2-chlorobenzyl)-4-(3-hydroxymethylphenyl)-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
5-(2-bromo-5-methylbenzyl)-4-phenyl-2,4-dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(2-chlorobenzyl)4-(4-methylphenyl)-2,4-dihydro-[1,2,4]triazole-3-thione;
5-(2,3-dichlorobenzyl)-4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(2-methylbenzyl)-4-phenyl-2,4-dihydro-[1,2,4]triazole-3-thione;
5-(2-chlorobenzyl)-4-(4-chlorophenyl)-2,4-dihydro-[1,2,4]triazole-3-thione;
5-(2-chlorobenzyl)-4-(2-chlorophenyl)-2,4-dihydro-[1,2,4]triazole-3-thione;
5-(2-chlorobenzyl)-4-phenyl-2,4-dihydro-[1,2,4]triazole-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(2-chloro-5-methoxybenzyl)4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(2,3-dimethoxybenzyl)-4-phenyl-2,4-dihydro-[1,2,4]triazole-3-thione;
4phenyl-5-(2,3,4-trimethoxybenzyl)-2,4-dihydro-[1,2,4]-triazole-3-thione;
   and pharmaceutically acceptable salts thereof.

A further aspect of the invention concerns the novel compounds of formula (Ia) for use as a medicament.

A further aspect of the invention concerns the novel compounds of formula (Ia) or a pharmaceutically acceptable salt thereof, in the manufacture of a medicament, for the treatment or prophylaxis of diseases or conditions in which inhibition of the enzyme MPO is beneficial.

According to the invention, we further provide a process for the preparation of the novel compounds of formula (I) or a pharmaceutically acceptable salt, enantiomer, diastereomer or racemate thereof which process [wherein variable groups are, unless otherwise specified, as defined in formula (I) above] comprises:
(a) reaction of a thiosemicarbazide derivative of formula (II) 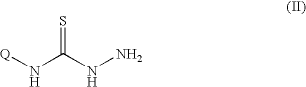
with an ester of formula (III) 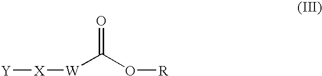
wherein R represents C1 to 6 alkyl; or
(b) reaction of a thiosemicarbazide derivative of formula (II), with a carboxylic acid of formula (IV) 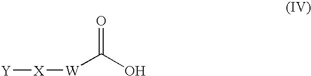
in the presence of a coupling agent; or
(c) reaction of a thiosemicarbazide derivative of formula (II), with an acyl chloride of formula (V) 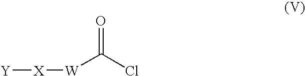
   or
(d) reaction of an isothiocyanate derivative of formula (VI) 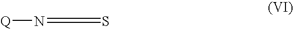
with an acid hydrazide of formula (VII) 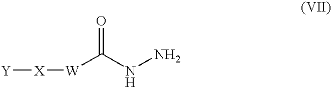
   or
(e) reaction of an isocyanate derivative of formula (VIII) 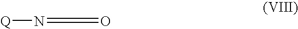
   with an acid hydrazide of formula (VII) 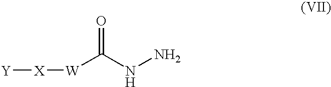
   followed by treatment of the intermediate 2,4-dihydro-[1,2,4]triazol-3-one with Lawesson's reagent;
   and where necessary converting the resultant compound of formula (I), or another salt thereof, into a pharmaceutically acceptable salt thereof; or converting the resultant compound of formula (I) into a further compound of formula (I) ; and where desired converting the resultant compound of formula (I) into an optical isomer thereof.

In process (a), the compounds of formulae (II) and (III) are reacted together in an organic solvent such as an alcohol, for example, methanol, in the presence of a base such as sodium methoxide, at a temperature between 25° C. and the reflux temperature of the reaction mixture until reaction is complete, typically for between 10 to 50 hours. See, for example, Pesson, M. et al. C. R. Hebd. Sceances Acad. Sci., 248; 1959; 1677-1679. The reaction mixture is then cooled and concentrated. The residue is dissolved in water and acidified with an acid such as acetic acid or hydrochloric acid, typically to pH about 3 to 6. The precipitate is collected and then purified by chromatography or recrystallization when necessary.

In process (b), the compounds of formulae (I) and (IV) are dissolved in an organic solvent such as dichloromethane, or DMF or mixtures thereof. A coupling reagent (for example, a peptide (amide) bond forming reagent) such as 1-(3-dimethylaminopropyl)-3-ethylcarbodiimide (EDC) is added at temperatures generally between 0 and 30° C. The reaction is stirred at temperatures between 10° C. and the reflux temperature of the solvent until the reaction is completed, typically for 1 to 15 h. The reaction mixture is concentrated and the residue is dissolved in a solvent, for example, a mixture of water and methanol with an added inorganic base such as sodium hydroxide or sodium hydrogen carbonate and heated to temperatures between 25° C. and the reflux temperature of the reaction rimxture is until the reaction is complete, typically for 30 minutes to 20 h. The reaction mixture is neutralized with an acid such as hydrochloric acid, and the precipitated product is collected by filtration. For reactions where the product does not precipitate, the reaction mixture is concentrated and the product is extracted with an organic solvent such as ethyl acetate or chloroform and the organic phase is dried and concentrated. The crude products are purified by chromatography or recrystallization when necessary.

In process (c), a compound of formula (V) in an organic solvent such as chloroform or dichloromethane containing a base such as pyridine or triethylamine is treated with a compound of formula (II). The reaction mixture is stirred at a temperature between 10° C. and the reflux temperature of the solvent until reaction is complete, typically for 1-16 h. The reaction mixture is concentrated and the residue is dissolved in a solvent such as water and methanol and the process is then continued as in process (b).

In process (d), the compounds of formulae (VI) and (VII) are dissolved in an organic solvent such as ethanol, isopropanol, DMF or dioxane or mixtures thereof, and then heated to between 25° C. and the reflux temperature of the solvent, preferably under an inert atmosphere until the reaction is completed, typically for 1 to 16 h. See, for example, Bamford, M. J. et al. J. Med. Chem. 1995, 38, 3502-3513; Abdelai, A. M. et al. Sci. Pharm. 1997, 65, 99-108; Petrovanu, M. Phosphorus, Sulphur and Silicon 1996, 108, 231-237. The reaction mixture is poured onto ice and the intermediate collected and, if necessary, purified by chromatography. If the intermediate does not precipitate, it is isolated by extraction with an organic solvent such as chloroform, ethyl acetate or diethyl ether. The intermediate is then dissolved in water or an alcohol or mixtures thereof, preferably with an added base such as, for example, sodium hydroxide or sodium hydrogen carbonate, and heated to between 25° C. and the reflux temperature of the solvent until the reaction is completed, typically for 1 to 16 h. The mixture is then neutralized by addition of an acid. Either the product precipitates upon neutralization, and it is then collected by filtration or the reaction mixture is extracted with an organic solvent. The crude product is then purified by chromatography or by recrystallization when necessary. In a particular embodiment, the compounds of formulae (VI) and (VII) are dissolved in an organic solvent such as ethanol, isopropanol, DNF or dioxane or mixtures thereof, and then heated in a microwave oven to a suitable temperature, generally between 120° C. and 150° C., for a suitable period of time, typically about 5 to 15 minutes. Under these conditions, the products of formula (I) may be formed directly without the need to isolate any intermediate.

In process (e), the compounds of formulae (VIII) and (VII) are reacted together using essentially the same conditions as for the reaction of compounds of formulae (VI) and (VII) in process (d), including in particular the use of microwave oven technology. The intermediate 2,4dihydro-[1,2,4]triazol-3-one is then converted into the corresponding 2,4-dihydro-[1,2,4]triazole-3-thione of formula (1) by treatment with Lawesson's reagent. Suitable conditions for the use of Lawesson's reagent will be readily apparent to the man skilled in the art. See, for example, Cava, M. P. et al, Tetrahedron, 1985, 41, 5061-5087. Thus, for example, the intermediate 2,4-dihydro-[1,2,4]triazol-3-one and Lawesson's reagent are dissolved or suspended in a suitable dry organic solvent such as benzene, toluene, xylene, tetrahydrofuran, dichloromethane or dioxane and then heated to between 30 ° C. and the reflux temperature of the solvent until reaction is complete, typically for between one to 30 hours. If the sulphurisation reaction is conducted in a microwave oven, then suitable temperatures are generally between 120° C. and 150° C. and suitable reaction times are generally about 10 minutes to 1 hour.

Compounds of formula (V) may be prepared by treatment of compounds of formula (IV) with thionyl chloride. See, for example, Encyclopedia of Reagents for Organic Synthesis, Vol. 7, ed. Paquette, L. A., John Wiley & Sons, West Sussex, 1995.

The present invention includes compounds of formula (I) in the form of salts, in particular acid addition salts. Suitable salts include those formed with both organic and inorganic acids. Such acid addition salts will normally be pharmaceutically acceptable although salts of non-pharmaceutically acceptable acids may be of utility in the preparation and purification of the compound in question. Thus, preferred salts include those formed from hydrochloric, hydrobromic, sulphuric, phosphoric, citric, tartaric, lactic, pyruvic, acetic, succinic, fumaric, maleic, methanesulphonic and benzenesulphonic acids. Salts of compounds of formula (I) may be formed by reacting the free base, or a salt, enantiomer or racemate thereof, with one or more equivalents of the appropriate acid. The reaction may be carried out in a solvent or medium in which the salt is insoluble or in a solvent in which the salt is soluble, for example, water, dioxan, ethanol, tetrahydrofuran or diethyl ether, or a mixture of solvents, which may be removed in vacuo or by freeze drying. The reaction may also be a metathetical process or it may be carried out on an ion exchange resin.

Compounds of formulae (II), (III), (IV), (VI), (VII) and (VIII) are either known in the literature or may be prepared using known methods that will be readily apparent to the man skilled in the art.

The compounds of the invention and intermediates thereto may be isolated from their reaction mixtures and, if necessary further purified, by using standard techniques.

The compounds of formula (I) may exist in enantiomeric forms. Therefore, all enantiomers, diastereomers, racemates and mixtures thereof are included within the scope of the invention. The various optical isomers may be isolated by separation of a racemic mixture of the compounds using conventional techniques, for example, fractional crystallisation, or HPLC. Alternatively, the various optical isomers may be prepared directly using optically active starting materials.

Intermediate compounds may also exist in enantiomeric forms and may be used as purified enantiomers, diastereomers, racemates or mixtures.

The compounds of formula (I) and their pharmaceutically acceptable salts are useful because they possess pharmacological activity as inhibitors of the enzyme MPO.

The compounds of formulae (I) and their pharmaceutically acceptable salts are indicated for use in the treatment or prophylaxis of diseases or conditions in which modulation of the activity of the enzyme myeloperoxidase (MPO) is desirable. In particular, linkage of MPO activity to disease has been implicated in neuroinflammatory diseases. Therefore the compounds of the present invention are particularly indicated for use in the treatment of neuroinflammatory conditions or disorders in mammals including man. Such conditions or disorders will be readily apparent to the man skilled in the art.

Conditions or disorders that may be specifically mentioned include multiple sclerosis, Alzheimer's disease, Parkinson's disease, amyotrophic lateral sclerosis and stroke, as well as other inflammatory diseases or conditions such as asthma, chronic obstructive pulmonary disease, cystic fibrosis, idiopathic pulmonary fibrosis, acute respiratory distress syndrome, sinusitis, rhinitis, psoriasis, dermatitis, uveitis, gingivitis, atherosclerosis, inflammatory bowel disease, renal glomerular damage, liver fibrosis, sepsis, proctitis, rheumatoid arthritis, and inflammation associated with reperfusion injury, spinal cord injury and tissue damage/scarring/adhesion/rejection. Lung cancer has also been suggested to be associated with high MPO levels. The compounds are also expected to be useful in the treatment of pain.

Prophylaxis is expected to be particularly relevant to the treatment of persons who have suffered a previous episode of, or are otherwise considered to be at increased risk of, the disease or condition in question. Persons at risk of developing a particular disease or condition generally include those having a family history of the disease or condition, or those who have been identified by genetic testing or screening to be particularly susceptible to developing the disease or condition.

For the above mentioned therapeutic indications, the dosage administered will, of course, vary with the compound employed, the mode of administration and the treatment desired. However, in general, satisfactory results are obtained when the compounds are administered at a dosage of the solid form of between 1 mg and 2000 mg per day.

The compounds of formulae (I) and pharmaceutically acceptable derivatives thereof, may be used on their own, or in the form of appropriate pharmaceutical compositions in which the compound or derivative is in admixture with a pharmaceutically acceptable adjuvant, diluent or carrier. Administration may be by, but is not limited to, enteral (including oral, sublingual or rectal), intranasal, inhalation, intravenous, topical or other parenteral routes. Conventional procedures for the selection and preparation of suitable pharmaceutical formulations are described in, for example, “Pharmaceuticals—The Science of Dosage Form Designs”, M. E. Aulton, Churchill Livingstone, 1988. The pharmaceutical composition preferably comprises less than 80% and more preferably less than 50% of a compound of formulae (I) or a pharmaceutically acceptable salt thereof.

There is also provided a process for the preparation of such a pharmaceutical composition which comprises mixing the ingredients.

The invention is illustrated, but in no way limited, by the following examples:

General Methods

All solvents used were analytical grade and commercially available anhydrous solvents were used for reactions. Reactions were typically run under an inert atmosphere of nitrogen or argon.

¹H and ¹³C NMR spectra were recorded at 400 MHz for proton and 100 MHz for carbon-13 either on a Varian Unity+ 400 NMR Spectrometer equipped with a 5 mm BBO probe with Z-gradients, or on a Bruker DPX400 NMR spectrometer equipped with a 4-nucleus probe equipped with Z-gradients; or at 600 MHz for proton and 150 MHz for carbon-13, on a Bruker DRX600 NMR Spectrometer equipped with a 5 mm BBO probe with Z-gradients or a 5 mm TXI probe with Z-gradients; or at 300 MHz for proton on a Bruker Avance DPX 300 spectrometer. Unless specifically noted in the examples, spectra were recorded at 400 MHz for proton and 100 MHz for carbon-13. The following reference signals were used: the middle line of DMSO-d₆ δ 2.50 (¹H), δ 39.51 (¹³C); the middle line of CD₃OD δ 3.31 (¹H) or δ 49.15 (¹³C); acetone-d₆ 2.04 (¹H), 206.5 (¹³C); and CDCl₃ δ 7.26 (¹H), the middle line of CDCl₃ δ 77.16 (¹³C) (unless otherwise indicated).

Mass spectra were recorded on a Waters LCMS consisting of an Alliance 2795 (LC) and a ZQ single quadrupole mass spectrometer. The mass spectrometer was equipped with an electrospray ion source (ESI) operated in a positive or negative ion mode. The capillary voltage was 3 kV and the mass spectrometer was scanned from m/z 100-700 with a scan time of 0.3 or 0.8 s. Separations were performed on either Waters X-Terra MS, C8-columns, (3.5 μm, 50 or 100 mm×2.1 mm i.d.), or a ScantecLab's ACE 3 AQ column (100 mm×2.1 mm i.d.). The column temperature was set to 40° C. A linear gradient was applied using a neutral or acidic mobile phase system, running at 0% to 100% organic phase in 4-5 minutes, flow rate 0.3 ml/min. Neutral mobile phase system: acetonitrile/[10 mM NH₄OAc (aq)/MeCN (95:5)], or [10 mM NH₄OAc (aq)/MeCN (1/9)]/[10 mM NH₄OAc (aq)/MeCN (9/1)]. Acidic mobile phase system: [133 mM HCOOH (aq)/MeCN (5/95)]/[8 mM HCOOH (aq)/MeCN (98/2)].

Alternatively, mass spectra were recorded on a Finnigan MAT SSQ7000 equipped with a thermo spray ion source (TSP) operated in the positive mode and scanning from m/z 120-600 with a scan time of 1 s. Samples were introduced via an isocratic pump, Shimatzu LC-10AD. The mobile phase was 50 mM ammonium acetate in 40:60 acetonitrile/MilliQ Water and the flow rate 1 ml/min; or on a Waters 2690 Separations Module with a Waters 2487 Dual λ Absorbance Detector and a Waters Micromass ZQ. Column: Chromolith Performance RP-18e, 4.6×100 mm, Mobile phase A: Acetonitrile, Mobile phase B: 0.1% formic acid (aq.), Flow: 2 ml/min, Injection volume: 20 μl, UV-Detection: 254 nm, Gradient: 30% A to 100% in 6 minutes. ZQ with ES−, ES+, MS 97-800, and Cone V30 was used.

HPLC analyses were performed on an Agilent HP1000 system consisting of G1379A Micro Vacuum Degasser, G1312A Binary Pump, G1367A Wellplate auto-sampler, G1316A Thermostatted Column Compartment and G1315B Diode Array Detector. Column: X-Terra MS, Waters, 4.6×50 mm, 3.5 μm. The column temperature is was set to 40° C. and the flow rate to 1.5 ml/min. The Diode Array Detector was scanned from 210-300 nm, step and peak width were set to 2 nm and 0.05 min, respectively. A linear gradient was applied, run from 0% to 100% acetonitrile, in 4 min. Mobile phase: acetonitrile/10 mM ammonium acetate in 5 % acetonitrile in MilliQ Water; or on a Gynkotek P580 HPG, gradient pump with a Gynkotek UVD 170S UV-Vis detector. Column: Chromolith Performance RP-18e, 4.6×100 mm, Mobile phase A: Acetonitrile, Mobile phase B: 0.1% trifluoroacetic acid (aq), Flow: 3 ml/min, Injection volume: 20 μl, Detection: 254 nm, Gradient: 10% A to 100% in 5.0 minutes.

A typical workup procedure after a reaction consisted in extraction of the product with a solvent such as ethyl acetate, washing with water followed by drying of the organic phase over MgSO₄ or Na₂SO₄ and concentration of the solution in vacuo.

Thin layer chromatography (TLC) was performed on Merck TLC-plates (Silica gel 60 F₂₅₄) and the spots were visualized by UV. Preparative layer chromatography was performed on Merck PLC-Plates (Silica gel 60 F₂₅₄, 2 mm). Merck Silica gel 60 (0.040-0.063 mm) was used for flash chromatography. Typical solvents used for flash chromatography were mixtures of chloroform/methanol, toluene/ethyl acetate and ethyl acetate/hexanes.

Preparative chromatography was run on a Gilson auto-preparative HPLC with a diode array detector. Column: XTerra MS C8, 19×300 mm, 7 μm. Gradient with acetonitrile/0.1M ammonium acetate in 5% acetonitrile in MilliQ Water, run from 20% to 60% acetonitrile, in 13 min. Flow rate: 20 ml/min. Alternatively, purification was achieved on a semi preparative Shimadzu LC-8A HPLC with a Shimadzu SPD-10A UV-vis.-detector equipped with a Waters Symmetry® column (C18, 5 μm, 100 mm×19 mm). Gradient with acetonitrile/0.1% trifluoroacetic acid in MilliQ Water, run from 35% to 60% acetonitrile in 20 min. Flow rate: 10 ml/min.

Recrystallization was typically performed in solvents or solvent mixtures such as 10 ether, ethyl acetate/heptanes and methanol/water.

The following abbreviations have been used: DMF=N,N-dimethylformamide; DMSO=dimethylsulfoxide; THF=tetrahydrofuran; EDC=1-(3-dimethylaminopropyl)-3-ethyl-carbodiimide; NCS=N-chlorosuccinimide; aq.=aqueous.

Starting materials used were either available from commercial sources or prepared as according to literature procedures and had experimental data in accordance to those reported. The following are examples of starting materials that were prepared:
(2,6-Dimethylphenyl)acetic acid: Löfgren, N. et al. Acta Chem. Scand. 1963,17,1252-1261.
(2-Chlorophenyl)acetic acid hydrazide: Rosen, G. M. et al. J. Heterocycl. Chem. 1971, 8, 659-662.
Pyrrole-2-carboxylic acid hydrazide: Chunchatprasert, L. et al. J. Chem. Res. Miniprint 1997, 1, 101-115.
(2-Bromo-5-methylphenyl)acetic acid: Lewis, E. E. et al. J. Org. Chem. 1940, 5, 290-299.
(2-Butoxyphenyl)-acetic acid: WO 92/09561.
Biphenyl-2-yl-acetic acid: v. Braun, J. et al. Justus Liebigs Ann. Chem. 1929, 468, 258-303.
4-Butoxyphenoxyacetic acid: Baker, B. R. et al. J. Med. Chem. 1972, 15, 940-44.
2-Hydroxy-N-methylbenzamide: Cohen, S. M. et al. J. Am. Chem. Soc. 1998, 120, 6277-6286.
(2-Methylcarbamoylphenoxy)acetic acid: Valcavi, U. Farmaco Ed. Sci. 1963, 18, 990-1000.
3-Butoxyphenoxyacetic acid: Baker, B. R. et al. J. Med. Pharm. Chem. 1960, 2, 633-657.
Methyl 3-(cyanomethyl)benzoate: Chand, P. et al. J. Med. Chem. 1997, 40, 4030-4052.

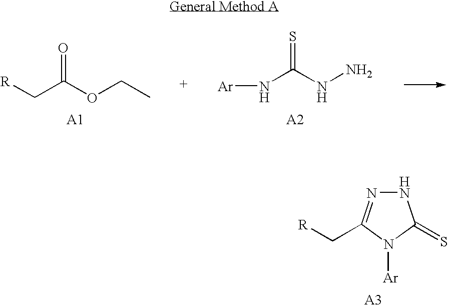

A solution of 1M sodium methoxide (1.3 to 4 equiv., freshly prepared from sodium metal and methanol) was added to a mixture of compound A1 (1.0-2.0 equiv.) and compound A2 (1.0 Q equiv.), optionally dissolved in MeOH (0-5 mL/100 mg A2). The reaction mixture was refluxed for 24 h. In case the reaction was not completed after 24 h (as monitored by TLC or LC-MS), more compound A1 and 1M sodium methoxide were added, and the reaction mixture was refluxed for up to an additional 45 h. The reaction mixture was cooled and concentrated, and then the residue was dissolved in water and acidified with acetic acid to pH about 5 to 6. The precipitate was collected and washed with water. The crude product was purified by chromatography or recrystallization when necessary. The pure product was dried in vacuo.

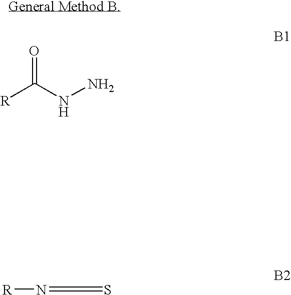

Compound B1 (1.0 equiv.) and compound B2 (1.5 to 2.5 equiv.) were dissolved in isopropanol (about 5 mL/100 mg B2) and refluxed under an argon atmosphere until the reaction was complete (monitored by LC-MS or TLC; typical reaction times 1 to 21 h). The reaction mixture was cooled and poured onto ice and the precipitate was collected and washed with water. The precipitated intermediate was dissolved in 2% aqueous sodium hydroxide (about 10 mL/100 mg B2) and refluxed for 2 h. The reaction mixture was cooled, neutralized with 1M hydrochloric acid and the precipitate was collected and purified by chromatography or recrystallization if necessary.

### General Method C

Compound B2 (1.1 equiv.) was added to a solution of compound B1 (1.0 equiv.) in isopropanol/DMF (about 3:1; about 5 ml100 mg B1). The reaction mixture was stirred at 60° C. until completion (monitored by LC-MS; reaction times were typically 1 to 2 h), and then it was cooled down, concentrated and purified by flash chromatography (typical eluent 1 to 3% methanol in chloroform) to give the condensation product. This intermediate was then dissolved in methanol/water (about 1:1; about 3 mL/100 mg intermediate), and sodium hydrogen carbonate (2 equiv.) was added. The reaction mixture was heated at 75° C. until completion (monitored by LC-MS; typical reaction times were 2 to 12 h). After cooling down, the reaction mixture was acidified to pH about 4 to 5 with 1% hydrochloric acid and the product precipitated. The precipitate was collected, washed with water, and dried in vacuo and purified when necessary.

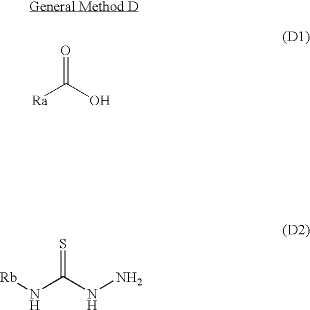

EDC (1.0 to 1.2 equiv.) was added to a solution of compound D1 (1.0 equiv.) and compound D2 (1.0 to 1.1 equiv.) in dichloromethane (about 2.5 mL/100 mg D2) at 0° C. In some cases DMF was added to dissolve the reactants. The resulting suspension/solution was stirred at ambient temperature and formed a clear solution as the reaction progressed. When the coupling reaction was complete as monitored by TLC or LC-MS (typical reaction times 1 to 4 h), the solvent was removed under reduced pressure. The residue was dissolved in a mixture of methanol and 2% aqueous sodium hydroxide (about 2:1; about 4.5 mL/100 mg D2) followed by heating at 70 to 75° C. for 1 to 20 h. The reaction mixture was allowed to attain ambient temperature and was neutralized with 1M hydrochloric acid. The precipitate was collected by filtration, washed with water and dried in vacuo. The crude product was purified by chromatography or recrystallization when necessary.

### General Method E

EDC (1.0 equiv.) was added to the solution of compound D1 (1.0 equiv.) and compound D2 (1.1 equiv.) in dichloromethane/DMF (about 2:1; about 2 mL/100 mg D1) at 0° C. The reaction mixture was stirred at ambient temperature until completion (monitored by LC-MS; reaction times were typically 4 to 14 h), concentrated and purified by column chromatography. The intermediate was then dissolved in methanol/water (about 1:1; about 3 mL/100 mg intermediate). NaHCO₃ (2 equiv.) was added and the reaction mixture was heated at 75° C. until completion (monitored by LC-MS; typical reaction times were 2 to 16 h). After cooling down, the reaction mixture was acidified with 1% hydrochloric acid (pH about 4 to 5) and the product precipitated. The precipitate was collected and purified by chromatography or recrystallization when necessary.

Except where otherwise indicated, the compounds of Examples 1 to 19 were prepared using the procedure of General Method A.

### EXAMPLE 1

### 5-(4-Aminobenzyl)-4-[3,5-di(trifluoromethyl)phenlyl]-2,4-dihydro-[1,2,4]triazole-3-thione

The title compound was obtained in 55% yield starting from ethyl (4-aminophenyl)acetate (130 mg, 726 μmol), 4-[3,5-di(trifluoromethyl)-phenyl]-3-thiosemicarbazide (200 mg, 660 μmol) and 1M NaOMe (845 μL).

¹H NMR (CDCl₃) δ 11.6 (1H, s), 7.96 (1H, s), 7.51 (2H, s), 6.58 (2H, d, J=8.3 Hz), 6.49 (2H, d, J=8.4 Hz), 3.79 (2H, s), 2.73 (2H, br s);

¹³C NMR (CDCl₃) δ 169.5, 151.9, 146.3, 135.1, 133.28 (q, J=34.5 Hz), 129.5, 129.2, 124.0, 122.3, 121.3, 115.6, 31.8;

MS (ESI) m/z 419 (M+1).

### EXAMPLE 2

### 5-(4-Chlorobenzyl)4-(4-methylphenyl)-2,4-dihydro-[1,2,4]triazole-3-thione

Starting with ethyl (4-chlorophenyl)acetate (201 mg, 1.1 mmol), 4-(4-methylphenyl)-3-thiosemicarbazide (200 mg, 1.1 mmol) and 1M NaOMe (1.25 mL) afforded 157 mg (45%) of the title compound.

¹H NMR (CDCl₃) δ 11.45 (1H, s), 7.28 (2H, d, J=8.1 Hz), 7.19 (2H, d, J=8.4 Hz), 6.98 (2H, d, J=8.3 Hz), 6.88 (2H, d, J=8.4 Hz), 3.81 (2H, s), 2.43 (3H, s);

¹³C NMR (CDCl₃) δ 169.4, 151.7, 140.7, 133.7, 132.5, 130.7, 130.6, 130.2, 129.1, 127.9, 31.7, 21.6;

MS (ESI) m/z 316 (M+1).

### EXAMPLE 3

### 5-Isobutyl4-phenyl-2,4-dihydro-[1,2,4]triazole-3-thione

The title compound was prepared according to method A with the exception that after concentration the mixture was refluxed for 2 h in 2% aqueous NaOH (5 mL). After cooling it was poured onto ice and neutralized with 1M aqueous HCl. The precipitate was filtered off and purified. Starting with ethyl 3-methylbutyrate (311 mg, 2.4 mmol), 4-phenyl-3-thiosemicarbazide (200 mg, 1.2 mmol) and 1M NaOMe (4.8 mL) gave 48 mg (17%) of the title compound.

¹H NMR (DMSO-d₆) δ 13.70 (1H, s), 7.56 (3H, m), 7.40 (2H, m), 2.32 (2H, d, J=7.1 Hz), 1.77-1.67 (1H, m), 0.79 (6H, d, J=6.7 Hz);

¹³C NMR (DMSO-d₆) δ 167.5, 151.3, 133.8, 129.4, 128.3, 128.3, 34.0, 25.5, 21.9;

MS (ESI) m/z 234 (M+1).

### EXAMPLE 4

### 5-(4-Hydroxybenzyl)-4-[3-(methylthio)phenyl]-2,4-dihydro-[1,2,4]triazole-3-thione

The title compound was obtained in 23% yield starting from ethyl (4-hydroxyphenyl)acetate (186 mg+50 mg, 1.0 mmol), 4-[3-(methylthio)-phenyl]-3-thiosemicarbazide (200 mg, 938 μmol), 1M NaOMe (1.10+0.1 mL) and MeOH (1 mL).

¹H NMR (DMSO-d₆) δ 13.76 (1H, s), 9.29 (1H, s), 7.38 (2H, m), 6.98 (2H, m), 6.69 (2H, d, J=8.5 Hz), 6.58 (2H, d, J=8.5 Hz), 3.72 (2H, s), 2.40 (3H, s);

¹³C NMR (DMSO-d₆) δ 167.8, 156.2, 151.5, 139.6, 134.4, 129.5, 129.5, 126.7, 125.2, 124.6, 124.57, 115.1, 30.6, 14.4;

MS (ESI) m/z 330 (M+1).

### EXAMPLE 5

### 5-(2,5-Dimethoxybenzyl)-4-[3-(methylthio)phenyl]-2,4-dihydro-[1,2,4]triazole-3-thione

Starting from ethyl (2,5-dimethoxyphenyl)acetate (210+50 μL, 1.30 mmol), 4-[3-(methylthio)-phenyl]-3-thiosemicarbazide (200 mg, 0.938 mmol), 1M NaOMe (1.10+1.18 mL) and MeOH (1+1 mL) afforded a total of 135 mg (39%) of the title compound.

¹H NMR (DMSO-d₆) δ 13.73 (1H, s), 7.39 (2H, m), 7.03 (2H, m), 6.77 (2H, m), 6.53 (1H, d, J=2.9 Hz), 3.75 (2H, s), 3.63 (3H, s), 3.54 (3H, s), 2.43 (3H, s);

¹³C NMR (DMSO-d₆) δ 167.6, 152.8, 151.1, 150.8, 139.7, 134.3, 129.5, 126.5, 125.0, 124.5, 123.6, 116.2, 112.7, 111.7, 55.7, 55.3, 26.0, 14.4;

MS (ESI) m/z 374 (M+1).

### EXAMPLE 6

### 5-(4-Hydroxybenzyl)-4-(4-iodophenyl)-2,4-dihydro-[1,2,4]triazole-3-thione

Starting with ethyl (4-hydroxyphenyl)acetate (135+50 mg, 1.03 mmol), 4-(4-iodophenyl)-3-thiosemicarbazide (200 mg, 0.682 mmol), 1M NaOMe (1.25+0.1+0.8 mL) and MeOH (1.2+1.2 mL) afforded 65 mg (23%) of the title compound.

¹H NMR (DMSO-d₆) δ 13.79 (1H, s), 9.29 (1H, s), 7.84 (2H, d, J=8.5 Hz), 7.03 (2H, d, J=8.5 Hz), 6.71 (2H, d, J=8.5 Hz), 6.57 (2H, d, J=8.5 Hz), 3.73 (2H, s);

¹³C NMR (DMSO-d₆) δ 167.7, 156.2, 151.3, 138.0, 133.7, 130.5, 129.5, 124.5, 115.1, 95.8, 30.6;

MS (ESI) m/z 410 (M+1).

### EXAMPLE 7

### 5-(2,5-Dimethoxybenzyl)-4-(4-iodophenyl)-2,4-dihydro-[1,2,4]triazole-3-thione

The title compound was obtained in 14% yield starting from ethyl (2,5-dimethoxyphenyl)acetate (150 μL, 0.751 mmol), 4-(4-iodophenyl)-3-thiosemicarbazide (200 mg, 0.682 mmol), 1M NaOMe (0.8+0.9 mL) and MeOH (1.2+1.2 mL).

¹H NMR (DMSO-d₆) δ 13.75 (1H, s), 7.85 (2H, d, J=8.5 Hz), 7.05 (2H, d, J=8.5 Hz), 6.76 (2H, m), 6.52 (1H, d, J=2.8 Hz), 3.75 (2H, s), 3.63 (3H, s), 3.53 (3H, s);

¹³C NMR (DMSO-₆) δ 167.8, 153.2, 151.4, 151.1, 138.5, 133.8, 130.8, 123.8, 116.6, 113.1, 112.0, 96.5, 56.1, 55.7, 26.4;

MS (TSP) m/z 454 (M+1).

### EXAMPLE 8

### 4-(4-Carboxyphenyl)-5-(2,4,6-trimethylbenzyl)-2,4-dihydro-[1,2,4]triazole-3-thione

Starting with ethyl (2,4,6-trimethylphenyl)acetate (215+50 mg, 1.30 mmol), 4-(4-carboxyphenyl)-3-thiosemicarbazide (200 mg, 0.947 mmol), 1M NaOMe (1.75+1.1 mL) and MeOH (1+1 mL) afforded 15 mg (4%) of the title compound.

¹H NMR (600 MHz, DMSO-d₆) δ 13.71 (1H, s), 13.23 (1H, s), 8.11 (2H, d, J=8.4 Hz), 7.61 (2H, d, J=8.4 Hz), 6.78 (2H, s), 3.66 (2H, s), 2.18 (3H, s), 2.07 (6H, s);

¹³C NMR (DMSO-d₆) δ 167.5, 166.6, 150.4, 137.2, 136.6, 135.7, 132.2, 130.4, 128.7, 128.6, 128.4, 25.9, 20.5, 19.6;

MS (ESI) m/z 354 (M+1).

### EXAMPLE 9

### 5-(4-Hydroxybenzyl)-4-[4-(piperidinosulfonyl)phenyl]-2,4-dihydro-[1,2,4]triazole-3-thione

Starting from ethyl (4-hydroxyphenyl)acetate (126+50 mg, 0.98 mmol), 4-[4-(piperidinosulfonyl)-phenyl]-3-thiosemicarbazide (200 mg, 0.636 mmol, obtained from Maybridge), 1M NaOMe (0.98+0.83 mL) and MeOH (1.2+1.2 mL) afforded 42 mg (15%) of the title compound.

¹H NMR (DMSO-d₆) δ 13.89 (1H, s), 9.30 (1H, s), 7.77 (2H, d, J=8.5 Hz), 7.50 (2H, d, J=8.5 Hz), 6.61 (2H, d, J=8.4 Hz), 6.50 (2H, d, J=8.5 Hz), 3.84 (2H, s), 2.89 (4H, t, J=5.2 Hz), 1.58 (4H, m), 1.45 (2H, m);

¹³C NMR (DMSO-d₆) δ 167.8, 156.1, 151.4, 137.5, 136.0, 129.5, 129.3, 128.3, 124.0, 115.1, 46.7, 30.7, 24.7, 22.9;

MS (ESI) m/z 431 (M+1).

### EXAMPLE 10

### 5-(2,5-Dimethoxybenzyl)-4-[4-(piperidinosulfonyl)phenyl]-2,4-dihydro-[1,2,4]triazole-3-thione

Starting with ethyl (2,5-dimethoxyphenyl)acetate (140 μL, 700 μmol), 4-[4-(piperidinosulfonyl)phenyl]-3-thiosemicarbazide (200 mg, 0.636 mmol), 1M NaOMe (0.73+0.83 mL) and MeOH (1.2+1.2 mL) afforded 115 mg (38%) of the title compound.

¹H NMR (DMSO-d₆) δ 13.84 (1H, s), 7.81 (2H, d, J=8.5 Hz), 7.56 (2H, d, J=8.5 Hz), 6.75 (2H, m), 6.52 (1H, d, J=2.8 Hz), 3.83 (2H, s), 3.62 (3H, s), 3.54 (3H, s), 2.92 (4H, t, J=5.1 Hz), 1.56 (4H, m); 1.40 (2H, m);

¹³C NMR (DMSO-d₆) δ 167.5, 152.8, 150.9, 150.8, 137.4, 136.5, 129.3, 128.3, 123.3, 116.2, 112.8, 111.7, 55.8, 55.4, 46.6, 26.2, 24.7, 22.8;

MS (ESI) m/z 475 (M+1).

### EXAMPLE 11

### 5-(2,4,6-Trimethylbenzyl)-4(4-sulfamoylphenyl)-2,4-dihydro-[1,2,4]triazole-3-thione

The title compound was prepared according to method A with the exception that it was refluxed for 11 days and then left at ambient temperature for 7 more days. Starting with ethyl (2,4,6-trimethylphenyl)acetate (46 mg, 0.22 mmol), 4-(4-sulfamoylphenyl)-3-thiosemicarbazide (50 mg, 0.20 mmol, obtained from Maybridge), 1M NaOMe (0.23+0.1+0.23 mL) and MeOH (1.8+1.8 mL) afforded 2 mg (3%).

¹H NMR (DMSO-d₆) δ 13.65 (1H, s), 7.99 (2H, d, J=8.5 Hz), 7.69 (2H, d, J=8.4 Hz), 7.57 (2H, s), 6.79 (2H, s), 3.65 (2H, s), 2.19 (3H, s), 2.08 (6H, s);

¹³C NMR (DMSO-d₆) δ 167.5, 149.7, 144.7, 136.5, 135.6, 129.2, 128.8, 128.4, 126.8, 26.0, 20.5, 19.6;

MS (ESI) m/z 389 (M+1).

### EXAMPLE 12

### 5-(4-Hydroxybenzyl)-4-(2,4,6-trichlorophenyl)-2,4-dihydro-[1,2,4]triazole-3-thione

The title compound was prepared according to method A with the exception that more ester, 1M NaOMe and MeOH were added after 7 days, the reaction was refluxed for 5 more days and then left at ambient temperature for 5 days. Starting with ethyl (4hydroxyphenyl)acetate (200+20 mg, 1.2 mmol), 4(2,4,6-trichlorophenyl)-3-thiosemicarbazide (200 mg, 0.74 mmol), 1M NaOMe (2.2+0.2 mL) and MeOH (0.8+0.4 mL) afforded 48 mg (17% yield).

¹H NMR (DMSO-d₆) δ 13.99 (1H, s), 9.34 (1H, s), 7.93 (2H, s), 6.72 (2H, d, J=8.4 Hz), 6.57 (2H, d, J=8.4 Hz), 3.68 (2H, s);

¹³C NMR (DMSO-d₆) δ 167.5, 156.5, 151.1, 136.2, 135.3, 129.9, 129.0, 128.1, 123.3, 115.2, 30.7;

MS (ESI) m/z 386/388 (M+1).

### EXAMPLE 13

### 4-[2-Chloro-5-(trifluoromethyl)phenyl]-5-(2,5-dimethoxybenzyl)-2,4-dihydro-[1,2,4]triazole-3-thione

The title compound was prepared according to method A with the exception that more ester, 1M NaOMe and MeOH were added after 7 days and then refluxed for 5 more days. Starting with ethyl (2,5-dimethoxyphenyl)acetate (223+20 μL, 1.2 mmol), 4-[2-chloro-5-(trifluoromethyl)phenyl]-3-thiosemicarbazide (200 mg, 0.74 mmol), 1M NaOMe (2.2+0.2 mL) and MeOH (0.8+0.4 mL) afforded 123 mg (39%).

¹H NMR (DMSO-d₆) δ 13.87 (1H, s), 7.89 (1H, dd, J=8.5 Hz, 2.0 Hz), 7.82 (1H, d, J=8.5 Hz), 7.77 (1H, d, J=2.0 Hz), 6.70 (2H, m), 6.45 (1H, m), 3.83 (1H, d, J=15.9 Hz), 3.75 (1H, d, J=15.9 Hz), 3.60 (3H, s), 3.45 (3H, s);

¹³C NMR (150 MHz, DMSO-d₆) δ 167.8, 152.8, 150.9, 150.8, 137.0, 132.0, 131.3, 128.3, 128.2, 124.1, 122.3, 116.2, 113.2, 111.5, 55.5, 55.3, 25.8;

MS (ESI) m/z 430 (M+1).

### EXAMPLE 14

### 4-(4Carboxyphenyl)-5-(diphenylmethyl)-2,4-dihydro-[1,2,4]triazole-3-thione

Starting with ethyl (diphenyl)acetate (250 mg, 1.04 mmol), 4-(4-carboxyphenyl)-3-thiosemicarbazide (200 mg, 947 μmol), 1M NaOMe (1.75+1.1 mL) and MeOH (1+1 mL) afforded 7 mg (2%) of the title compound.

¹HNMR (DMSO-d₆) δ 7.81 (2H, d, J=8.4 Hz), 7.23 (6H, m), 7.11 (4H, m), 7.01 (2H, d, J=8.4 Hz), 5.20 (1H, s);

¹³C NMR (DMSO-d₆) δ 168.1, 167.5, 153.0, 138.7, 134.4, 129.6, 128.6, 128.4, 127.5, 127.1, 47.7;

MS (ESI) m/z 388 (M+1).

### EXAMPLE 15

### 5-(2-Bromobenzyl)4(4-sulfamoylphenyl)-2,4-dihydro-[1,2,4]triazole-3-thione

The title compound was prepared according to method A with the exception that it was left at reflux for 7 days after the addition of more ester and sodium methoxide and after that at ambient temperature for 7 more days. Starting with ethyl (2-bromophenyl)acetate (74.3 mg, 306, μmol), 4-(4-sulfamoylphenyl)-3-thiosemicarbazide (50 mg, 203 μmol), 1M NaOMe (0.23+0.1+0.23 mL) and MeOH (1.8+1.8 mL) afforded 10 mg (12%).

¹H NMR (DMSO-d₆) δ 7.93 (2H, d, J=8.6 Hz), 7.57 (2H, d, J=8.6 Hz), 7.53 (1H, d, J=8.1 Hz), 7.31-7.15 (3H, m), 3.93 (2H, s);

¹³C NMR (DMSO-d₆) δ 167.7, 149.7, 144.7, 136.6, 134.3, 132.4, 131.5, 129.2, 129.0, 127.8, 126.7, 123.9, 32.3;

MS (ESI) m/z 425/427 (M+1).

### EXAMPLE 16

### 5-(4-Hydroxybenzyl)-4-(naphthalen-1-yl)-2,4-dihdyro-[1,2,4]triazole-3-thione

The title compound was prepared according to method A with the exception that it was refluxed for 7 days without further addition of ester or sodium methoxide. Starting with ethyl (4-hydroxyphenyl)acetate (249 mg, 1.4 mmol), 4-(naphthalen-1-yl)-3-thiosemicarbazide (200 mg, 920 μmol), 1M NaOMe (2.8 mL) and MeOH (0.2 mL) afforded 53 mg (17%).

¹H NMR (DMSO-d₆) δ 13.90 (1H, s), 9.17 (1H, s), 8.11 (1H, d, J=8.1 Hz), 8.03 (1H, d, J=8.1 Hz), 7.58 (2H, m), 7.44 (1H, t, J=7.8 Hz), 7.37 (1H, d, J=7.6 Hz), 7.08 (1H, d, J=8.6 Hz), 6.50 (2H, d, J=8.0 Hz), 6.40 (2H, d, J=8.6 Hz), 3.58 (1H, d, J=16.2 Hz), 3.50 (1H, d, J=15.6 Hz);

¹³C NMR (DMSO-d₆) δ 168.4, 156.1, 152.3,133.7, 130.2, 129.8, 129.4, 129.3, 128.3, 127.4, 127.3, 126.6, 125.5, 124.1, 122.0, 114.9, 30.6;

MS (ESI) m/z 334 (M+1).

### EXAMPLE 17

### 5-(4-Hydroxybenzyl)-4-(2,6-dibromo-4-methylphenyl)-2,4-dihydro-[1,2,4]triazole-3-thione

The title compound was prepared according to method A with the exception that more ester, 1M NaOMe and MeOH were added after 7 days, refluxed for 7 more days and left at ambient temperature for 1 month. Starting with ethyl (4-hydroxyphenyl)acetate (159+40 mg, 1.1 mmol), 4-(2,6-dibromo-4-methylphenyl)-3-thiosemicarbazide (200 mg, 0.59 mmol, obtained from Maybridge), 1M NaOMe (1.8+0.2 mL) and MeOH (0.2+0.4 mL) afforded 10 mg (4%).

¹H NMR (DMSO-d₆) δ 13.80 (1H, s), 9.28 (1H, s), 7.67 (2H, s), 6.73 (2H, d, J=8.6 Hz), 6.58 (2H, d, J=8.6 Hz), 3.58 (2H, s), 2.39 (3H, s);

¹³C NMR (DMSO-d₆) δ 167.3, 156.4, 150.9, 143.9, 133.1, 130.0, 128.9, 123.9, 123.5, 115.1, 30.9, 20.1;

MS (ESI) m/z 456 (M+1).

### EXAMPLE 18

### 5-(4-Hydroxybenzyl)-4-(3,4,5-trimethoxyphenyl)-2,4-dihydro-[1,2,4]triazole-3-thione

The title compound was prepared according to method A with the exception that it was refluxed for 4 days without further addition of ester or sodium methoxide. Starting with ethyl (4-hydroxyphenyl)acetate (210 mg, 1.2 mmol), 4-(3,4,5-trimethoxyphenyl)-3-thiosemicarbazide (200 mg, 0.78 mmol), 1M NaOMe (2.3 mL) and MeOH (0.7 mL) afforded 122 mg (42%).

¹H NMR (DMSO-d₆) δ 13.69 (1H, s), 9.27 (1H, s), 6.69 (2H, d, J=8.6 Hz), 6.57 (2H, d, J=8.6 Hz), 6.42 (2H, s), 3.75 (2H, s), 3.69 (3H, s), 3.64 (6H, s);

¹³C NMR (DMSO-d₆) δ 167.9, 156.1, 152.9, 151.9, 137.9, 129.6, 129.1, 124.7, 115.0, 106.2, 60.1, 56.0, 30.7;

MS (ESI) m/z 374 (M+1).

### EXAMPLE 19

### 5-(2,5-Dimethoxybenzyl)-4-(3,4,5-trimethoxyphenyl)-2,4-dihydro-[1,2,4,]triazole-3-thione

The title compound was prepared according to method A with the exception that it was refluxed for 4 days without further addition of ester or sodium methoxide. Starting with ethyl (2,5-dimethoxyphenyl)acetate (233 μL, 1.2 mmol), 4-(3,4,5-trimethoxyphenyl)-3-thiosemicarbazide (200 mg, 0.78 mmol), 1M NaOMe (2.3 mL) and MeOH (0.7 mL) afforded 106 mg (33%).

¹H NMR (DMSO-d₆) δ 13.66 (1H, s), 6.74 (2H, m), 6.47 (3H, m), 3.81 (2H, s), 3.68 (3H, s), 3.66 (6H, s), 3.61 (3H, s), 3.54 (3H, s);

¹³C NMR (DMSO-d₆) δ 167.7, 152.8, 151.3, 150.8, 137.7, 129.1, 123.7, 116.2, 112.4, 111.6, 105.9, 59.9; 56.0, 55.7, 55.2, 26.0;

MS (ESI) m/z 418 (M+1).

Except where otherwise indicated, the compounds of Examples 20 to 34 were prepared using the procedure of General Method B.

### EXAMPLE 20

### 5-(4-Methoxyphenoxymethyl)-4-(2-tetrahydrofuran-2-yl-methyl)-2,4-dihydro-[1,2,4]triazole-3-thione

The title compound was obtained in 68% yield starting from (4-methoxyphenoxy)acetic acid hydrazide (100 mg, 510 μmol) and 2-tetrahydrofurfurylisothiocyanate (110 mg, 765 μmol) in isopropanol (5.0 mL).

¹H NMR (DMSO-₆) δ 13.85 (1H, s), 6.99 (2H, d, J=9.1 Hz), 6.88 (2H, d, J=9.1 Hz), 5.16 (2H, s), 4.21 (2H, m), 3.99 (1H, m), 3.74 (4H, m), 3.61 (1H, m), 1.95 (1H, m), 1.80 (2H, m), 1.68 (1H, m);

¹³C NMR (DMSO-d₆) δ 167.6, 154.1,151.2, 148.7, 116.1, 114.7, 75.6, 67.4, 61.0, 55.3, 47.2, 28.2, 25.1;

MS (ESI) m/z 322 (M+1).

### EXAMPLE 21

### 5-(4-Methoxyphenoxymethyl)-4-(3-methoxypropyl)-2,4-dihydro-[1,2,4]triazole-3-thione

Starting from (4-methoxyphenoxy)acetic acid hydrazide (100 mg, 510 μmol) and 3-methoxypropylisothiocyanate (100 mg, 765 μmol) gave 65 mg (41% yield) of the title compound.

¹H NMR (DMSO-d₆) δ 13.82 (1H, s), 6.99 (2H, m), 6.89 (2H, m), 5.13 (2H, s), 4.04 (2H, t, J=7.5 Hz), 3.70 (3H, s), 3.36 (2H, t, J=6.0 Hz), 3.20 (3H, s), 1.97 (2H, m);

¹³C NMR (DMSO-d₆) δ 167.3, 154.2, 151.1, 148.2, 116.1, 114.7, 69.0, 60.6, 57.8, 55.4, 41.3, 27.5;

MS (ESI) m/z 310 (M+1).

### EXAMPLE 22

### 5-(4-Methoxyphenoxymethyl)-4-(2-phenylethyl)-2,4-dihydro-[1,2,4]triazole-3-thione

Starting from (4-methoxyphenoxy)acetic acid hydrazide (100 mg, 510 μmol) and 2-phenylethylisothiocyanate (125 mg, 765 μmol) gave 152 mg (87% yield) of the title compound.

¹H NMR (DMSO-d₆) δ 13.88 (1H, s), 7.33-7.23 (3H, m), 7.17 (2H, m), 6.96 (2H, m), 6.89 (2H, m), 4.88 (2H, s), 4.18 (2H, t, J=7.8 Hz), 3.70 (3H, s), 3.03 (2H, t, J=7.8 Hz);

¹³C NMR (DMSO-d₆) δ 167.3, 154.2, 151.0, 148.2, 137.8, 128.7, 128.6, 126.7, 115.9, 114.7, 60.3, 55.4, 45.2 33.2;

MS (ESI) m/z 342 (M+1).

### EXAMPLE 23

### 5-(4-Methoxyphenoxymethyl)-4-(3-morpholin-4-yl-propyl)-2,4-dihydro-[1,2,4]triazole-3-thione

The title compound was prepared according to method B with the exception that after reflux in isopropanol, the reaction was poured onto ice and the mixture was concentrated. A precipitate formed upon standing at 4° C. for 12 h. Starting from (4-methoxyphenoxy)acetic acid hydrazide (100 mg, 510 μmol) and 3-morpholinopropylisothiocyanate (142 mg, 765 μmol) gave 122 mg (66%) of the title compound.

¹H NMR DMSO-d₆) δ 13.81 (1H, s), 6.99 (2H, m), 6.90 (2H, m), 5.15 (2H, s), 4.02 (2H, t, J=7.5 Hz), 3.70 (3H, s), 3.46 (4H, br t, J=3.9 Hz), 2.30 (6H, m), 1.92 (2H, m);

¹³C NMR (DMSO-d₆) δ 167.3, 154.2, 151.2, 148.3, 115.9, 114.7, 66.0, 60.5, 55.4, 54.9, 53.0, 42.1, 23.8;

MS (ESI) m/z 365 (M+1).

### EXAMPLE 24

### 4-Butyl-5-[(4-methoxyphenylamino)-methyl]-2,4dihydro-[1,2,4]triazole-3-thione

The title compound was prepared according to method B with the exception that after the reaction was completed, the neutralized water phase was extracted with chloroform (3×). Starting from (4-methoxyphenylamino)acetic acid hydrazide (100 mg, 512 μmol, obtained from Zelinsky Institute) and butylisothiocyanate (88.5 mg, 769 μmol) gave 43 mg (29%) of the title compound.

¹H NMR (DMSO-d₆) δ 13.56 (1H, s), 6.73 (2H, m), 6.61 (2H, m), 5.84 (1H, br t, J=5.9 Hz), 4.30 (2H, d, J=5.9 Hz), 3.95 (2H, br t, J=7.8 Hz), 3.63 (3H, s), 1.62 (2H, m), 1.30 (2H, m), 0.87 (3H, t, J=7.4 Hz);

¹³C NMR (DMSO-d₆) δ 166.9, 151.3, 150.4, 141.8, 114.6, 113.4, 55.3, 43.0, 29.6, 19.4, 13.6;

MS (ESI) m/z 293 (M+1).

### EXAMPLE 25

### 5-[(4-Methoxyphenylamino)-methyl](3-methoxypropyl)-2,4-dihydro-[1,2,4]triazole-3-thione

The title compound was prepared according to method B with the exception that it did not precipitate upon pouring onto ice, but was concentrated in vacuo prior to the next step. After the reaction, the neutralized water phase was extracted with chloroform (3×). Starting from (4-methoxyphenylamino)acetic acid hydrazide (100 mg, 512 μmol) and 3-methoxypropylisothiocyanate (101 mg, 769 μmol) gave 106 mg (67%) of the title compound.

¹H NMR (DMSO-d₆) δ 13.58 (1H, s), 6.73 (2H, m), 6.62 (2H, m), 5.81 (1H, t, J=6.1 Hz), 4.29 (2H, d, J=5.9 Hz), 4.02 (2H, t, J=7.4 Hz), 3.63 (3H, s), 3.45 (2H, t, J=6.1 Hz), 3.22 (3H, s), 1.92 (2H, m);

¹³C NMR (DMSO-d₆) δ 166.9, 151.4, 150.5, 141.8, 114.6, 113.5, 69.0, 57.8, 55.3, 40.9, 27.3;

MS (ESI) m/z 309 (M+1).

### EXAMPLE 26

### 4-Hexyl-5-[(4-methoxyphenylamino)methyl]-2,4-dihydro-[1,2,4]triazole-3-thione

The title compound was prepared according to method B with the exception that after the reaction, the neutralized water phase was extracted with chloroform (3×). Starting with (4-methoxyphenylamino)acetic acid hydrazide (100 mg, 512 μmol) and hexylisothiocyanate (110 mg, 769 μmol) gave 62 mg (38%) of the title compound.

¹H NMR (DMSO-d₆) δ 13.56 (1H, s), 6.73 (2H, m), 6.61 (2H, m), 5.84 (1H, br s), 4.30 (2H, s), 3.93 (2H, m), 3.63 (3H, s), 1.63 (2H, m), 1.26 (6H, m) 0.84 (3H, m);

¹³C NMR (DMSO-d₆) δ 166.9, 151.3, 150.4, 141.8, 114.6, 113.4, 55.2, 43.3, 30.7, 27.4, 25.7, 21.9, 13.8;

MS (ESI) m/z 321 (M+1).

### EXAMPLE 27

### 5-(2-Chlorobenzyl)-4-(2-ethoxyphenyl)-2,4-dihydro-[1,2,4]triazole-3-thione

Starting with (2-chlorophenyl)acetic acid hydrazide (100 mg, 542 μmol) and 2-ethoxyphenylisothiocyanate (97.1 mg, 812 μmol) gave 128 mg (68%) of the title compound.

¹H NMR (DMSO-d₆) δ 13.67 (1H, s) 7.46 (1H, m), 7.35 (1H, m), 7.27-7.13 (5H, m), 7.02 (1H, m), 4.05-3.91 (2H, m), 3.91 (1H, d, J=16.5 Hz), 3.81 (1H, d, J=16.5 Hz), 1.19 (3H, t, J=7.0 Hz);

¹³C NMR (DMSO-d₆) δ 168.2, 153.8, 150.6, 133.2, 132.2, 131.3, 131.3, 130.1, 129.1, 128.9, 127.1, 121.7, 120.5, 113.4, 63.8, 29.2, 14.4;

MS (ESI) m/z 346 (M+1).

### EXAMPLE 28

### 5-(2-Chlorobenzyl)-4-(3-acetylphenyl)-2,4-dihydro-[1,2,4]triazole-3-thione

Starting with (2chlorophenyl)acetic acid hydrazide (100 mg, 542 μmol) and 3-acetylphenylisothiocyanate (235 mg, 1.3 mmol) gave 89 mg (48%) of the title compound.

¹H NMR (DMSO-d₆) δ 13.86 (1H, s), 8.04-(1H, d, J=7.7 Hz), 7.84 (1H, s), 7.65 (1H, t, J=7.8 Hz), 7.59 (1H, m), 7.34-7.23 (1H, m), 7.20 (3H, m), 3. 99 (2H, s), 2.50 (3H, s);

¹³C NMR (DMSO-d₆) δ 196.9, 167.9, 150.2, 137.8, 133.9, 133.0, 132.9, 132.3, 131.3, 129.9, 129.2, 129.1, 129.0, 127.9, 127.2, 29.5, 26.8;

MS (ESI) m/z 344 (M+1).

### EXAMPLE 29

### 5-(2-Chlorobenzyl)-4-(4-fluorophenyl)-2,4-dihydro-[1,2,4]triazole-3-thione

The title compound was prepared according to method B with the exception that the first reaction step was performed at ambient temperature and with the addition of methanol (2 10 ml) during 2% NaOH treatment. Starting with (2-chlorophenyl)acetic acid hydrazide (0.10 g, 0.54 mmol) and 4-fluorophenylisothiocyanate (0.12 g, 0.81 mmol) afforded 0.14 g (81%) of the title compound.

¹H NMR (DMSO-d₆) δ 14.0 (1H, s), 7.64-7.36 (8H, m), 4.17 (2H, s);

¹³C NMR (DMSO-d₆) δ 168.7, 162.9 (d, J=246 Hz), 151.0, 133.8, 133.0, 132.0, 131.3, 131.2, 130.4, 129.9, 129.7, 127.9, 117.0 (d, J=23 Hz), 30.2;

MS (ESI) m/z 320 (M+1).

### EXAMPLE 30

### 5-(2-Chlorobenzyl)-4-pyridin-3-yl-2,4-dihydro-[1,2,4]triazole-3-thione

The title compound was prepared according to method B with the exception that the first reaction step was performed at ambient temperature for 17 h in dioxane (3 ml) and that methanol (10 ml) was added during 2% NaOH treatment. Starting with (2-chlorophenyl)acetic acid hydrazide (0.10 g, 0.54 mmol) and 3-pyridylisothiocyanate (0.090 mL, 0.81 mmol) afforded 0.13 g (81%) of the title compound.

¹H NMR (DMSO-d₆) δ 14.0 (1H, s), 8.72 (1H, m), 8.57 (1H, d, J=2.3 Hz), 7.86 (1H, m), 7.61 (1H, m), 7.40 (1H, m), 7.34-7.22 (3H, m), 4.06 (2H, s);

¹³C NMR (DMSO-d₆) δ 168.6, 150.7, 150.6, 149.2, 136.5, 133.4, 132.5, 131.7, 130.9, 129.6, 129.5; 127.7, 124.6, 29.8;

MS (ESI) m/z 303 (M+1).

### EXAMPLE 31

### 5-(2-Chlorobenzyl)-4-(2-methoxy-5-methylphenyl)-2,4-dihydro-[1,2,4]triazole-3-thione

The title compound was prepared according to method B with the exception that in the second step 2% NaOH (10 mL) and MeOH (2 mL) were used and the reaction was refluxed for 1 h. Starting from (2-chlorophenyl)acetic acid hydrazide (100 mg, 542 μmol) and 2-methoxy-5-methylphenylisothiocyanate (146 mg, 812 μmol) gave 98 mg (52%) of the title compound.

¹H NMR (DMSO-d₆) δ 13.67 (1H, s), 7.35 (1H, m), 7.28-7.13 (3H, m), 7.12 (1H, m), 7.05 (1H, d, J=8.5 Hz), 6.92 (1H, d, J=1.8 Hz), 3.88 (1H, d, J=16.4 Hz), 3.79 (1H, d, J=16.4 Hz), 3.64 (3H, s), 2.22 (3H, s);

¹³C NMR (DMSO-d₆) δ 168.2, 152.4, 150.6, 133.2, 132.2, 131.6, 131.3, 130.1, 129.6, 129.0, 128.8, 127.0, 121.2, 112.4, 55.7, 29.1, 19.8;

MS (ESI) m/z 346 (M+1).

### EXAMPLE 32

### 5-(4-Methoxyphenoxymethyl)-4-cyclopropyl-2,4-dihydro-[1,2,4]triazole-3-thione

The title compound, 112 mg (79%), was obtained starting from 4-methoxyphenoxyacetic acid hydrazide (100 mg, 510 μmol) and cyclopropylisothiocyanate (76 mg, 765 μmol).

¹H NMR (DMSO-d₆) δ 13.66 (1H, s), 6.99 (2H, m), 6.87 (2H, m), 5.11 (2H, s), 3.69 (3H, s), 2.99 (1H, m), 1.13 (2H, m), 1.00 (2H, m);

¹³C NMR (DMSO-d₆) δ 169.0, 154.1, 151.3, 149.6, 116.2, 114.6, 60.7, 55.3, 25.7, 6.4;

MS (ESI) m/z 278 (M+1).

### EXAMPLE 33

### 4-(2,2-Dimethoxyethyl)-5-[(4-methoxyphenylamino)methyl]-2,4-dihydro-[1,2,4]triazole-3-thione

The title compound was prepared according to method B with the exception that after the reaction was complete, the neutralized water phase was extracted with chloroform (3×). Starting with (4-methoxyphenylamino)acetic acid hydrazide (100 mg, 512 μmol) and 2,2-dimethoxyethylisothiocyanate (113 mg, 769 μmol) gave 51 mg (31%) of the title compound.

¹H NMR (DMSO-d₆) δ 13.64 (1H, s), 6.71 (2H, d, J=8.5 Hz), 6.59 (2H, d, J=8.6 Hz), 5.80 (1H, br s), 4.66 (1H, t, J=5.3 Hz), 4.30 (2H, s), 4.11 (2H, d, J=5.5 Hz), 3.62 (3H, s), 3.34 (6H, s);

¹³C NMR (DMSO-d₆) δ 167.2, 151.4, 150.9, 141.8, 114.5, 113.6, 101.3, 55.3, 55.1, 45.2;

MS (ESI) m/z 325 (M+1).

### EXAMPLE 34

### 5-(2-Chlorobenzyl)-4-(-3-methoxycarbonyl)phenyl-2,4-dihydro-[1,2,4]triazole-3-thione

The title compound was prepared according to method B with the exception of performing the first reaction at ambient temperature for 4.5 h in dioxane (5 mL) and the addition of methanol (10 ml) during 2% NaOH treatment. The crude product was isolated by concentration of the organic layers after partition of the reaction mixture between water and ethyl acetate. Starting with (2-chlorophenyl)acetic acid hydrazide (0.10 g, 0.54 mmol) and 3-isothiocyanatobenzoic acid methyl ester (0.16 g, 0.80 mmol) afforded 61 mg (17%) of the title compound.

¹H NMR (DMSO-d₆) δ 13.7 (1H, s), 7.92 (1H, m), 7.69-7.44 (3H, m), 7.23-7.02 (4H, m), 3.84 (2H, s), 3.73 (3H, s);

¹³C NMR (DMSO-d₆) δ 168.3, 165.5, 150.5, 134.2, 133.4, 132.6, 131.7, 131.2, 130.5, 130.4, 129.5, 129.4, 129.3, 127.6, 52.8, 29.9;

MS (ESI) m/z 360 (M+1).

Except where otherwise indicated, the compounds of Examples 35 to 42 were prepared using the procedure of General Method C.

### EXAMPLE 35

### 5-(2-Chlorobenzyl)-4-(2-methoxyphenyl)-2,4-dihydro-[1,2,4]triazole-3-thione

The title compound was prepared according to method C, with the exception that only isopropanol was used as a solvent in the first condensation step, and that in the second step acetonitrile was added to dissolve the intermediate. Starting from (2-chlorophenyl)acetic, acid hydrazide (96 mg, 0.52 mmol) and 2-methoxyphenylisbthiocyanate (94.5 mg, 0.57 mmol) afforded 96 mg (55%) of the title compound.

¹H NMR (CDCl₃) δ 11.38 (1H, br s), 7.44 (1H, dt, J=7.8 Hz, 1.8 Hz), 7.25 (1H, m), 7.18-7.06 (4H, m), 7.00 (2H, m), 3.94 (1H, d, J=16.4 Hz), 3.88 (1H, d, J=16.4 Hz), 3.72 (3H, s);

¹³C NMR (CDCl₃) δ 169.2, 154.7, 151.6, 134.1, 131.8, 131.8, 130.8, 129.9, 129.5, 128.8, 126.8, 121.5, 121.1, 112.4, 55.7, 29.6;

MS (ESI) m/z 332 (M+1), 330 (M−1).

### EXAMPLE 36

### 5-(2-Chlorobenzyl)-4-(3-methylphenyl)-2,4-dihydro-[1,2,4]triazole-3-thione

Starting from (2-chlorophenyl)acetic acid hydrazide (89 mg, 0.48 mmol) and 3-methylphenylisothiocyanate (79 mg, 0.53 mmol), gave the title compound in 72% yield.

¹H NMR (CD₃OD) δ 7.34 (1H, m), 7.27 (2H, m), 7.17 (2H, m), 7.06 (1H, dd, J=7.2 Hz, 1.6 Hz), 6.96 (1H, br d, J=7.6 Hz), 6.88 (1H, br s), 3.97 (2H, s), 2.31 (3H, s); ¹³C NMR (CDCl₃) δ 169.3; 152.1, 140.9, 135.0, 134.3, 133.1, 131.8, 131.6, 130.4, 129.8, 129.4, 127.9, 125.9, 30.7, 21.5;

MS (ESI) m/z 316 (M+1).

### EXAMPLE 37

### 5-[(2-Chlorophenyl)hydroxymethyl]-4-isobutyl-2,4-dihydro-[1,2,4]triazole-3-thione

Starting from (2-chlorophenyl)hydroxyacetic acid hydrazide (157 mg, 0.79 mmol) and 2-methylpropylisothiocyanate (99 mg, 0.86 mmol) gave the title compound in 37% yield.

¹H NMR (CD₃OD) δ 7.61 (1H, d, J=7.6 Hz), 7.34-7.23 (3H, m), 6.07 (1H, s), 3.90 (1H, dd, J=14 Hz, 8.0 Hz), 3.79 (1H, dd, J=14.0 Hz, 7.6 Hz), 3.31 (1H, br s), 2.43 (1H, septet, J=7.0 Hz), 0.92 (6H, dd, J=6.4 Hz, 3.6 Hz);

¹³C NMR (CDCl₃) δ 167.9, 152.8, 136.2, 132.2, 129.6, 129.2, 128.3, 127.1, 64.2, 51.0, 27.4, 19.7, 19.5;

MS (ESI) m/z 298 (M+1), 296 (M−1).

### EXAMPLE 38

### 5-[(2-Chloro-phenyl)hydroxymethyl]-4-cyclooctyl-2,4-dihydro-[1,2,4]triazole-3-thione

### a) (2-Chlorophenyl)hydroxyacetic acid hydrazide

To a solution of 2-chloromandelic acid (1.21 g, 6.49 mmol) and hydrazine hydrate (346 μL, 7.14 mmol) in CH₂Cl₂, EDC was added at 0° C. After stirring at this temperature for 3 h the product was collected by filtration and dried in vacuo to yield 880 mg (67%) of the title compound.

MS (ESI) m/z 201 (M+1), 199 (M−1).

### b) 5-[(2-Chloro-phenyl)hydroxymethyl]-4-cyclooctyl-2,4-dihydro-[1,2,4]triazole-3-thione

Starting from (2-chlorophenyl)hydroxyacetic acid hydrazide (120 mg, 0.60 mmol) and cyclooctylisothiocyanate (112 mg, 0.67 mmol) gave the title compound in 40% yield.

¹H NMR (CDCl₃) δ 12.03 (1H, br s), 7.58 (1H, m), 7.44 (1H, m), 7.28 (2H, m), 6.28 (1H, s), 4.2-4.0 (1H, m), 2.8-2.5 (1H, m), 1.3-1.8 (14H, m);

MS (ESI) m/z 352 (M+1), 350 (M−1).

### EXAMPLE 39

### 5-[(2-Chlorophenyl)hydroxymethyl]-4-(2,2-dimethoxyyethyl)-2,4-dihydro-[1,2,4]triazole-3-thione

The title compound was obtained in 49% yield starting from (2-chlorophenyl)hydroxyacetic acid hydrazide (143 mg, 0.72 mmol) and isothiocyanatoacetaldehyde dimethyl acetal (116 mg, 0.79 mmol).

¹H NMR (CD₃OD) δ 7.53-7.42 (1H, m), 7.20-7.02 (3H, m), 6.07 (1H, s), 4.62-4.51 (1H, m), 4.22-4.08 (1H, m), 4.06-3.89 (1H, m), 3.36-3.17 (6H, m);

¹³C NMR (CDCl₃) δ 167.6, 153.1, 136.0, 132.2, 129.4, 129.1, 128.3, 126.9, 101.9, 63.8, 56.2, 55.8, 46.2;

MS (ESI) m/z 330 (M+1), 328 (M−1).

### EXAMPLE 40

### 5-[(2-Chlorophenyl)hydroxymethyl]-4-(2-methylbutyl)-2,4-dihydro-[1,2,4]triazole-3-thione

Starting from (2-chlorophenyl)hydroxyacetic acid hydrazide (169 mg, 0.84 mmol) and 2-methylbutyl isothiocyanate (120 mg, 0.93 mmol) gave the title compound in 58% yield.

¹H NMR (CD₃OD) δ 7.65 (1H, m), 7.36-7.25 (3H, m), 6.10 (1H, s), 4.03-3.81 (2H, m), 2.28-2.15 (1H, m), 1.49-1.36 (1H, m), 1.30-1.16 (1H, m), 0.91 (6H, m);

¹³C NMR (CD₃OD) δ 168.4, 153.5, 137.0, 132.8, 130.1, 129.8, 128.9, 127.6, 64.8, 50.4, 34.4, 27.3, 16.9, 11.5;

MS (ESI) m/z 312 (M+1), 310 (M−1).

### EXAMPLE 41

### 5-(2-chlorobenzyl)-4-(3-methoxyphenyl)-2,4-dihydro-[1,2,4]triazole-3-thione

The title compound was prepared according to method C, with the exception that in the second step acetonitrile was added to dissolve the intermediate. Starting from (2-chlorophenyl)acetic acid hydrazide (104 mg, 0.56 mmol) and (3-methoxyphenyl)isothiocyanate (102 mg, 0.62 mmol) gave the title compound in 82% yield.

¹H NMR (CDCl₃) δ 11.61 (1H, br s), 7.39 (1H, t, J=8.0 Hz), 7.30 (1H, m), 7.19 (2H, m), 7.12 (1H, m), 7.03 (1H, dd, J=8.4 Hz, 2.4 Hz), 6.80 (1H, br d, J=7.6 Hz), 6.69 (1H, br s), 3.97 (2H, s), 3.74 (3H, s);

¹³C NMR (CDCl₃) δ 168.9, 160.5, 150.9, 134.1, 134.0, 131.8, 130.7, 130.5, 129.7, 129.0, 127.0, 119.9, 116.4, 113.3, 55.5, 29.9;

MS (ES]) m/z 332 (M+1), 330 (M−1).

### EXAMPLE 42

### 5-(2-Chlorobenzyl)-4-(2-methylphenyl)-2,4-dihydro-[1,2,4]triazole-3-thione

The title compound was prepared according to method C, with the exception that only isopropanol was used as a solvent in the first condensation step. Starting from (2-chlorophenyl)acetic acid hydrazide (90 mg, 0.49 mmol) and (2-methylphenyl)isothiocyanate (80 mg, 0.54 mmol) gave the title compound in 60% overall yield.

¹H NMR (DMSO-d₆) δ 13.79 (1H, br s), 7.43 (1H, m), 7.35 (3H, m), 7.29-7.18 (3H, m), 7.11 (1H, dd, J=7.6 Hz, 1.2 Hz), 3.89 (1H, d, J=16.4 Hz), 3.82 (1H, d, J=16.4 Hz), 1.88 (3H, s);

¹³C NMR (CDCl₃) δ 167.5, 150.6, 136.4, 133.9, 131.7, 131.2, 131.0, 130.7, 130.3, 129.2, 128.7, 127.8, 127.1, 126.7, 29.5, 16.8;

MS (ESI) m/z 316 (M+1), 314 (M−1).

### EXAMPLE 43

### 4-Phenyl-5-(pyrrol-2-yl)-2,4-dihydro-[1,2,4]triazole-3-thione

Pyrrole-2-carboxylic acid hydrazide (0.10 g, 0.80 mmol) and phenylisothiocyanate (0.16 g, 1.2 mmol) were suspended in 1,4-dioxane (3 mL). The resulting reaction mixture was stirred at ambient temperature for 3.5 h and then poured onto ice. The precipitate was collected by filtration and washed with water. Recrystallization afforded 84 mg (43%) of the title compound.

¹H NMR (CDCl₃) δ 11.89 (1H, br s), 9.58 (1H, s), 7.63 (3H, m), 7.42 (2H, m), 6.87 (1H, m), 6.04 (1H, m), 5.48 (1H, m);

¹³C NMR (CDCl₃) δ 169.3, 146.6, 134.7, 131.0, 130.5, 129.0, 122.2, 117.1, 112.2, 110.4;

MS (ESI) m/z 243 (M+1).

### EXAMPLE 44

### 5-(2-Chlorobenzyl)-4-(3-hvdroxymethylphenyl)-2,4-dihydro-[1,2,4]triazole-3-thione

### a) 4-(3-Hydroxymethyl)phenyl-3-thiosemicarbazide

(3-Hydroxymethyl)phenylisothiocyanate (0.47 g, 2.9 mmol, obtained from Organix Inc.) was dissolved in absolute ethanol (1.5 mL). Hydrazine hydrate (0.20 mL) diluted with water (0.20 mL) was added dropwise. The reaction mixture was stirred at ambient temperature for 1 h. Water (25 mL) was added to the resulting white paste and the mixture was neutralized with 2M HCl. The white precipitate was collected by filtration, washed with water and dried giving 0.20 g of product. A second crop of product was obtained by concentrating the filtrate to dryness. Yield: 0.37 g (66%). The crude product was used without further purification.

MS (ESI) m/z 198 (M+1).

### b) 5-(2-Chlorobenzyl)-4-(3-hydroxymethylphenyl)-2,4-dihydro-[1,2,4]triazole-3-thione

The title compound was prepared according to the method D with the exception that the crude product was extracted with dichloromethane from the neutralized reaction mixture. Starting from (2-chlorophenyl)acetic acid (0.17 g, 0.99 mmol) and 4-(3-hydroxymethylphenyl)-3-thiosemicarbazide (0.22 g, 1.1 mmol) afforded 32 mg (10%) of product.

¹H NMR (Acetone-d₆) δ 12.6 (1H, br s), 7.46 (2H, m), 7.35-7.21 (5H, m), 7.19 (1H, m), 4.66 (2H, s), 4.00 (2H, s);

¹³C NMR (Acetone-d₆) δ 151.8, 145.7, 135.3, 135.0, 134.1, 132.6, 130.6, 130.4, 130.2, 128.6, 128.4, 127.3, 110.9, 64.3;

MS (ESI) m/z 332 (M+1).

Except where otherwise indicated, the compounds of Examples 45 to 62 were prepared using the procedure of General Method D.

### EXAMPLE 45

### 5-(4-Chlorobenzyl)-4-phenyl-2,4-dihydro-[1,2,4]triazole-3-thione

Starting from (4-chlorophenyl)acetic acid (0.34 g, 2.0 mmol) and 4-phenyl-3-thiosemicarbazide (0.33 g, 2.0 mmol) afforded 0.53 g (88%) of the title compound.

¹H NMR (DMSO-d₆) δ 13.78 (1H, s), 7.48 (3H, m), 7.26 (4H, m), 6.97 (2H, m), 3.85 (2H, s);

¹³C NMR (DMSO-d₆) δ 168.1, 151.1, 133.7, 133.6, 131.7, 130.7, 129.6, 129.4, 128.4, 128.4, 30.9;

MS (ESI) m/z 302 (M+1).

### EXAMPLE 46

### 5-(2-Chlorobenzyl)-4-(3-chlorophenyl)-2,4-dihydro-[1,2,4]triazole-3-thione

Starting from (2-chlorophenyl)acetic acid (0.34 g, 2.0 mmol) and 4-(3-chlorophenyl)-3-thiosemicarbazide (0.40 g, 2.0 mmol) afforded 0.57 g (85%) of the title compound.

¹H NMR (DMSO-d₆) δ 13.63 (1H, br s), 7.58-7.43 (3H, m), 7.36-7.15 (5H, m), 3.78 (2H, s);

¹³C NMR (DMSO-d₆) δ 167.9, 150.1, 134.7, 133.4, 133.1, 132.2, 131.3, 130.9, 129.6, 129.2, 129.0, 128.3, 127.2, 127.2, 29.4;

MS (ESI) m/z 336 (M+1).

### EXAMPLE 47

### 5-(2-Fluorobenzyl)-4-phenyl-2,4-dihydro-[1,2,4]triazole-3-thione

Starting from (2-fluorophenyl)acetic acid (0.31 g, 2.0 mmol) and 4-phenyl-3-thiosemicarbazide (0.33 g, 2.0 mmol) afforded 0.16 g (27%) of the desired product.

¹H NMR (DMSO-d₆) δ 13.77 (1H, br s), 7.45 (3H, m), 7.29-7.18 (3H, m), 7.02 (3H, m), 3.84 (2H, s);

¹³C NMR (DMSO-d₆) δ 168.3, 160.5 (d, J=245 Hz), 150.6, 133.8, 131.5 (d, J=3.8 Hz), 129.8, 129.7, 129.6 (d, J=8.3 Hz), 128.5, 124.7 (d, J=3.6 Hz), 121.8 (d, J=15 Hz), 115.5 (d, J=21 Hz), 25.4 (d, J=3.6 Hz);

MS (ESI) m/z 286 (M+1).

### EXAMPLE 48

### 4-Phenyl-5-pyridin-3-yl-methyl-2,4-dihydro-[1,2,4]triazole-3-thione

Starting from pyridin-3-yl-acetic acid (0.27 g, 2.0 mmol) and 4-phenyl-3-thiosemicarbazide (0.33 g, 2.0 mmol) afforded 0.10 g (19%) of the title compound.

¹H NMR (DMSO-d₆) δ 13.73 (1H, s), 8.33 (1H, s), 8.10 (1H, s), 7.55-7.35 (4H, m), 7.32-7.16 (3H, m), 3.83 (2H, s);

¹³C NMR (DMSO-d₆) δ 168.4, 151.2, 150.1, 148.4, 136.8, 133.8, 130.6, 129.9, 129.7, 128.6, 123.7, 29.2;

MS (ESI) m/z 269 (M+1).

### EXAMPLE 49

### 5-(3-Chlorobenzyl)-4-(2-methoxyphenyl)-2,4-dihydro-[1,2,4]triazole-3-thione

Starting with (3-chlorophenyl)acetic acid (0.34 g, 2.0 mmol) and 4-(2-methoxyphenyl)-3-thiosemicarbazide (0.40 g, 2.0 mmol) afforded 0.16 g (24%) of the title compound.

¹H NMR (DMSO-₆) δ 13.8 (1H, s), 7.55 (1H, m), 7.29 (2H, m), 7.20 (2H, m), 7.09 (1H, m), 6.96 (2H, m), 3.85 (2H, s), 3.65 (3H, s);

¹³C NMR (DMSO-d₆) δ 168.7, 154.8, 151.5, 137.2, 133.1, 131.8, 130.4, 128.9, 127.7, 127.1, 121.9, 120.9, 112.9, 55.9, 31.2;

MS (ESI) m/z 332 (M+1).

### EXAMPLE 50

### 5-(3-Methoxybenzyl)-4-phenyl-2,4-dihydro-[1,2,4]triazole-3-thione

Starting from (3-methoxyphenyl)acetic acid (0.33 g, 2.0 mmol) and 4-phenyl-3-thiosemicarbazide (0.33 g, 2.0 mmol) afforded 95 mg (16%) of the title compound.

¹H NMR (DMSO-d₆) δ 13.77 (1H, s), 7.48 (3H, m), 7.22 (2H, m), 7.10 (1H, t, J=7.9 Hz), 6.73 (1H, dd, J=8.3 Hz, 2.0 Hz), 6.49 (1H, br d, J=7.6 Hz), 6.42 (1H, m), 3.82 (2H, s), 3.62 (3H, s);

¹³C NMR (DMSO-d₆) δ 168.3, 159.5, 151.5, 136.4, 133.9, 129.7, 129.6, 128.6, 121.1, 114.6, 112.9, 55.3, 31.8;

MS (ESI) m/z 298 (M+1).

### EXAMPLE 51

### 5-(2-Bromo-5-methylbenzyl)-4-phenyl-2,4-dihydro-[1,2,4]triazole-3-thione

Starting from (2-bromo-5-methylphenyl)acetic acid (0.46 g, 2.0 mmol) and 4-phenyl-3-thiosemicarbazide (0.33 g, 2.0 mmol) afforded 0.40 g (55%) of the title compound.

¹H NMR (DMSO-d₆) δ 13.8 (1H, s), 7.61 (3H, m), 7.49-7.38 (3H, m), 7.05 (2H, m), 3.95 (2H, s), 2.27 (3H, s);

¹³C NMR (DMSO-d₆) 67 168.2, 150.6, 137.6, 134.1, 133.9, 132.5, 132.4, 130.2, 129.9, 129.8, 128.6, 120.9, 32.5, 20.6;

MS (ESI) m/z 361 (M+1).

### EXAMPLE 52

### 5-(2-Bromobenzyl)-4-phenyl-2,4-dihydro-[1,2,4]triazole-3-thione

Starting from (2-bromophenyl)acetic acid (0.43 g, 2.0 mmol) and 4-phenyl-3-thiosemicarbazide (0.33 g, 2.0 mmol) afforded 0.58 g (84%) of the desired product.

¹H NMR (DMSO-d₆) δ 13.7 (1H, s), 7.43 (4H, m), 7.28-7.17 (3H, m), 7.11 (2H, m), 3.82 (2H, s);

¹³C NMR (DMSO-d₆) δ 168.2, 150.6, 134.6, 133.8, 132.8, 131.9, 129.9, 129.8, 129.6, 128.5, 128.1, 124.3, 32.6;

MS (ESI) m/z 347 (M+1).

### EXAMPLE 53

### 5-(2-Chloro-6-fluoro-3-methylbenzyl)-4-phenyl-2,4-dihydro-[1,2,4]triazole-3-thione

Starting from (2-chloro-6-fluoro-3-methylphenyl)acetic acid (242 mg, 1.2 mmol) and 4-phenyl-3-thiosemicarbazide (200 mg, 1.2 mmol) yielded 82 mg (21%) of the title compound.

¹H NMR (DMSO-d₆) 67 13.75 (1H, s), 7.54 (3H, m), 7.39 (2H, m), 7.19 (2H, m), 3.91 (2H, s), 2.16 (3H, d, J=1.7 Hz);

¹³C NMR (DMSO-d₆) δ 167.9, 159.2 (d, J=247.1 Hz), 149.4, 133.4, 131.5 (d, J=5.2 Hz), 131.1 (d, J=6.3 Hz), 129.6, 129.5, 129.5, 128.1, 128.1, 124.6 (d, J=3.7 Hz), 123.5 (d, J=18.3 Hz), 120.2 (d, J=19.0 Hz), 23.6 (d, J=4.0 Hz), 13.9 (d, J=3.3 Hz);

MS (ESI) m/z 334 (M+1).

### EXAMPLE 54

### 5-(Furan-2-ylmethyl)-4phenyl-2,4-dihydro-[1,2,4]triazole-3-thione

Starting with (furan-2-yl)acetic acid (151 mg, 1.2 mmol, obtained from Adv. Synthesis) and 4-phenyl-3-thiosemicarbazide (200 mg, 1.2 mmol) yielded 112 mg (36%) of the title compound.

¹H NMR (DMSO-d₆) δ 13.82 (1H, s), 7.49 (4H, m), 7.29 (2H, m), 2.27 (1H, m), 5.88 (1H, d, J=3.0 Hz), 3.95 (2H, s);

¹³C NMR (DMSO-d₆) δ 168.0, 148.9, 147.5, 142.3, 133.4, 129.4, 129.2, 129.2, 128.1, 128.1, 110.6, 107.8, 25.0;

MS (ESI) m/z 258 (M+1).

### EXAMPLE 55

### 5-(3-Methylbenzyl)-4-phenyl-2,4-dihydro-[1,2,4]triazole-3-thione

The title compound was synthesized in 59% yield starting from (3-methylphenyl)acetic acid (135 mg, 0.89 mmol) and 4-phenyl-3-thiosemicarbazide (150 mg, 0.89 mmol).

¹H NMR (CDCl₃) δ 12.21 (1H, s), 7.42-7.28 (4H, m), 7.09-6.98 (4H, m), 6.66 (2H, m), 3.18 (2H, s) 2.22 (3H, s);

¹³C NMR (CDCl₃) δ 168.7, 151.9, 138.4, 133.6, 133.3, 130.0, 129.6, 129.4, 128.5, 128.2, 128.1, 125.7, 32.1, 21.2;

MS (ESI) m/z 282 (M+1).

### EXAMPLE 56

### 5-(2-Chlorobenzyl)-4-(4-methylphenyl)-2,4-dihydro-[1,2,4]triazole-3-thione

Starting from (2-chlorophenyl)acetic acid (188 mg, 1.10 mmol) and 4-(4-methylphenyl)-3-thiosemicarbazide (200 mg, 1.10 mmol) afforded the title compound in 72% yield.

¹H NMR (CDCl₃+a few drops of CD₃OD) δ 7.28 (3H, m), 7.18 (2H, m), 77.08 (3H, m), 3.94 (2H, s), 2.41 (3H, s);

¹³C NMR (CDCl₃+a few drops of CD₃OD) δ 168.7, 151.0, 140.4, 134.1, 132.0, 130.7, 130.6, 130.5, 129.7, 129.0, 127.6, 127.1, 29.9, 21.3;

MS (ESI) m/z 316 (M+1).

### EXAMPLE 57

### 5-(2-Hydroxy-1-phenylethyl)-4-phenyl-2,4-dihydro-[1,2,4]triazole-3-thione

The title compound was synthesized in 50% yield starting from 2-hydroxymethyl-2-phenylacetic acid (250 mg, 1.50 mmol) and 4-phenyl-3-thiosemicarbazide (301 mg, 1.80 mmol). The product was extracted with chloroform since no precipitate was formed after adding HCl.

¹H NMR (CDCl₃) δ 13.01 (1H, s), 7.41-7.12 (7H, m), 6.87 (3H, m), 4.31 (1H, m), 3.99 (1H, dd, J=4.8 Hz, 9.2 Hz), 3.87 (1H, m), 3.74 (1H, br s);

¹³C NMR (CDCl₃) δ 168.3, 153.0, 135.4, 132.8, 129.9, 129.4, 128.8, 128.3, 128.1, 127.9, 64.3, 46.2;

MS (ESI) m/z 298 (M+1).

### EXAMPLE 58

### 5-(3,5-Dimethylbenzyl)-4-phenyl-2,4-dihydro-[1,2,]triazole-3-thione

The title compound was synthesized in 60% yield starting from (3,5-dimethylphenyl)acetic acid (150 mg, 0.91 mmol) and 4-phenyl-3-thiosemicarbazide (183 mg, 1.09 mmol).

¹H NMR (CDCl₃) δ 12.46 (1H, s), 7.24 (3H, m), 7.06 (2H, m), 6.81 (1H, s), 6.44 (2H, s), 3.77 (2H, s), 2.17 (6H, s);

¹³C NMR (CDCl₃) 168.6, 152.0, 138.2, 133.4, 133.3, 129.9, 129.5, 128.9, 128.2, 126.5, 32.0, 21.1;

MS (ESI) m/z 296 (M+1).

### EXAMPLE 59

### 5-(2,3-Dichlorobenzyl)-4-phenyl-2,4-dihydro-[1,2,4]triazole-3-thione

The title compound was synthesized in 70% yield starting from (2,3-dichlorophenyl)acetic acid (200 mg, 0.97 mmol) and 4-phenyl-3-thiosemicarbazide (196 mg, 1.20 mmol).

¹H NMR (DMSO-d₆) δ 13.81 (1H, s), 7.52 (4H, m), 7.37 (2H, m), 7.26 (1H, t, J=7.8 Hz), 7.20 (1H, dd, J=1.7 Hz, 7.8 Hz), 3.99 (2H, s);

¹³C NMR (DMSO-d₆) δ 167.8, 149.9, 135.1, 133.4, 131.7, 130.0, 129.6, 129.4, 128.1, 128.0, 30.5;

MS (ESI) m/z 336 (M+1).

### EXAMPLE 60

### 5-(2-Methylbenzyl)-4-(2-methoxyphenyl)-2,4-dihydro-[1,2,4]triazole-3-thione

Starting from (2-methylphenyl)acetic acid (200 mg, 1.33 mmol) and 4-(2-methoxy)-3-thiosemicarbazide (262 mg, 1.33 mmol) gave 135 mg (33%) of the title compound.

¹H NMR (CDCl₃) δ 11.96 (1H, s), 7.45 (1H, m), 7.25-6.96 (6H, m), 6.80 (1H, d, J=7.2 Hz), 3.81 (1H, d, J=16.0 Hz), 3.71 (1H, d, J=16.0 Hz), 3.66 (3H, s), 2.05 (3H, s);

¹³C NMR (CDCl₃) δ168.9, 168.7, 154.8, 152.2, 136.6, 132.0, 131.8, 130.3, 129.9, 129.7, 127.4, 126.0, 121.1, 112.3, 55.8, 29.8, 19.3;

MS (ESI) m/z 312 (M+1).

### EXAMPLE 61

### 5-(2,6-Dimethylbenzyl)-4phenyl-2,4-dihydro-[1,2,4]triazole-3-thione

Starting with (2,6-dimethylphenyl)acetic acid (196 mg, 1.2 mmol) and 4-phenyl-3-thiosemicarbazide (200 mg, 1.2 mmol) yielded 133 mg (38%) of the title compound.

¹H NMR DMSO-d₆) δ0 13.62 (1H, s), 7.57 (3H, m), 7.46 (2H, m), 7.03 (1H, m), 6.96 (2H, m), 3.68 (2H, s), 2.10 (6H, s);

¹³C NMR (DMSO-d₆) δ 167.7, 150.4, 136.7, 133.7, .131.8, 129.6, 129.5, 128.3, 127.7, 126.8, 26.3, 19.7;

MS (ESI) m/z 296 (M+1).

### EXAMPLE 62

### 5-(3-Trifluoromethylbenzyl)-4-phenyl-2,4-dihydro-[1,2,4]triazole-3-thione

Starting with (3-trifluoromethyl)acetic acid (0.41 g, 2.0 mmol) and 4-phenyl-3-thiosemicarbazide (0.33 g, 2.0 mmol) afforded 0.19 g (28%) of the title compound.

¹H NMR (DMSO-d₆) δ 13.8 (1H, s), 7.54-7.40 (5H, m), 7.34-7.22 (4H, m), 3.95 (2H, s);

¹³C NMR (DMSO-d₆) δ 168.4, 151.2, 136.2, 133.8, 133.4, 129.8, 129.7, 129;6, 128.6, 129.3 (q, J=31.6 Hz), 126.0 (q, J=3.8 Hz), 124.4 (q, J=272.3 Hz), 124.0 (q, J=3.7 Hz), 31.5;

MS (ESI) m/z 336 (M+1).

### EXAMPLE 63

### 5-Phenoxy-4-phenyl-2,4-dihydro-[1,2,4]triazole-3-thione

To a solution of 4-phenyl-3-thiosemicarbazide (200 mg, 1.19 mmol) in acetonitrile (6 mL), phenyl chlorothionoformate (233 μL, 1.68 mmol) was added at ambient temperature. After stirring at ambient temperature for 2.5 days, the reaction mixture was diluted with brine (15 mL) and extracted with ethyl acetate (3×20 mL). Purification afforded 79 mg (24%) of the title compound.

¹H NMR (DMSO-d₆) δ 10.16 (1H, br s), 7.56-7.45 (4H, m), 7.32 (5H, m), 6.99 (1H, t, J=7.4 Hz);

¹³C NMR (DMSO-d₆) δ 164.0, 159.9, 155.7, 140.4, 130.2, 129.1, 125.8, 121.8, 118.9, 117.2;

MS (ESI) m/z 270 (M+1), 268 (M−1).

Except where otherwise indicated, the compounds of Examples 64 to 72 were prepared using the procedure of General Method E.

### EXAMPLE 64

### 5-(2-Methylbenzyl)-4-phenyl-2,4-dihydro-[1,2,4]triazole-3-thione

Starting from (2-methylphenyl)acetic acid (336 mg, 2.23 mmol) and 4-phenyl-3-thiosemicarbazide (411 mg, 2.46 mmol) gave the title compound in 23% yield.

¹H NMR (CDCl₃) δ 12.93 (1H, br s), 7.46-7.36 (3H, m), 7.09-6.95 (5H, m), 6.76 (1H, app d, J=7.2 Hz), 3.76 (2H, s), 1.95 (3H, s);

¹³C NMR (CDCl₃,) δ 168.3, 151.4, 136.2, 133.2, 132.0, 130.3, 129.9, 129.6, 129.0, 127.9, 127.4, 126.0, 29.4, 19.0;

MS (ESI) m/z 282 (M+1), 280 (M−1).

### EXAMPLE 65

### 5-[(2-Chlorophenyl)hydroxymethyl]-4-cyclohexyl-2,4-dihydro-[1,2,4]triazole-3-thione

The title compound was prepared according to method E, with exception that no DMF was used in the coupling step. The product was obtained in 56% yield starting from (2-chlorophenyl)hydroxyacetic acid (363 mg, 1.94 mmol) and 4-hexyl-3-thiosemicarbazide (337 mg, 1.94 mmol).

¹H NMR (CDCl₃) δ 11.71 (1H, br s), 7.61 (1H, m), 7.43 (1H, m), 7.21 (2H, m), 6.20 (1H, s), 4.85 (1H, br s), 4.33 (1H, br s), 1.90-1.77 (3H, m), 1.73-1.55 (3H, m), 1.37-1.14 (4H, m);

¹³C NMR (CDCl₃) δ 166.2, 153.9, 135.5, 132.2, 130.2, 129.9, 128.3, 127.2, 65.0, 58.3, 29.1, 25.9, 24.8;

MS (ESI) m/z 324 (M+1).

### EXAMPLE 66

### 5-[(2-Chlorophenyl)hydroxymethyl]-4-phenyl-2,4-dihydro-[1,2,4]triazole-3-thione

Starting from (2-chlorophenyl)hydroxyacetic acid (360 mg, 1.96 mmol) and 4-phenyl-3-thiosemicarbazide (323 mg, 1.93 mmol) gave the title compound in 78% yield.

¹H NMR (DMSO-d₆) δ 13.90 (1H, s), 7.62-7.52 (4H, m), 7.40-7.27 (5H, m), 6.53 (1H, d, J=12.8 Hz), 5.59 (1H, s);

¹³C NMR (DMSO-d₆) δ 168.2, 152.3, 137.1, 133.4, 130.9, 129.5, 129.4, 129.3, 128.8, 128.4, 128.3, 127.0, 63.4;

MS (ESI) m/z 318 (M+1).

### EXAMPLE 67

### 5-[(2-Chlorophenyl)hydroxymethyl]-4-(3-methoxypropyl)-2,4-dihydro-[1,2,4]triazole-3-thione

The title compound was prepared according to method E, with the exception that DMF was is used as solvent in the coupling step. The product was isolated in 36% yield starting from (2-chlorophenyl)hydroxyacetic acid (355 mg, 1.90 mmol) and 4-(3-methoxypropyl)-3-thiosemicarbazide (311 mg, 1.90 mmol).

¹H NMR (CDCl₃) δ 11.55 (1H, s), 7.69 (1H, dd, J=7.6 Hz, 2.0 Hz), 7.37 (1H, dd, J=7.6 Hz, 1.2 Hz), 7.31-7.21 (2H, m), 6.15 (1H, s), 4.82 (1H, s), 4.26 (1H, ddd, J=14.1 Hz, 8.1 Hz, 6.3 Hz), 4.06 (1H, ddd, J=14.1 Hz, 8.1 Hz, 6.3 Hz), 3.47 (2H, t, J=6.0 Hz), 3.36 (3H, s), 2.21-2.01 (2H, m);

¹³C NMR (CDCl₃) δ 167.4, 153.2, 135.5, 132.2, 129.8, 129.6, 128.2, 127.3, 69.2, 64.3, 58.5, 41.8, 27.3;

MS (ESI) m/z 314 (M+1), 312 (M−1).

### EXAMPLE 68

### 5-(2-Chlorobenzyl)-4-(2-piperidin-1-yl-ethyl)-2,4-dihydro-[1,2,4]triazole-3-thione

The title compound was prepared according method E, with the exception that DMF was used as solvent in the coupling step. The product was obtained in 24% yield starting from (2-chlorophenyl)acetic acid (250 mg, 1.46 mmol) and 4-(2-pipenidinoethyl)-3-thiosemicarbazide (296 mg, 1.46 mmol).

hu 1H NMR (CDCl₃) δ 7.42 (1H, m), 7.24 (2H, m), 7.11 (1H, m), 4.26 (2H, s), 4.01 (2H, t, J=6.4 Hz), 2.62 (2H, t, J=6.4 Hz), 2.44 (4H, m), 1.57 (4H, m), 1.43 (2H, m);

¹³C NMR (CDCl₃) δ 167.6, 151.3, 133.9, 132.1, 130.3, 129.8, 129.1, 127.3, 56.3, 55.0, 42.4, 29.6, 26.0, 24.1;

MS (ESI) m/z 337 (M+1), 335 (M−1).

### EXAMPLE 69

### 4Butyl-5-(2-chlorobenzyl)-2,4-dihydro-[1,2,4]triazole-3-thione

The title compound was prepared according to method E, with the exception that dichloromethane was used as solvent in the coupling step. Starting from (2-chlorophenyl)acetic acid (489 mg, 2.86 mmol) and 4-butyl-3-thiosemicarbazide (464 mg, 3.15 mmol) gave the title compound in 16% yield.

¹H NMR (CDCl₃) δ 11.53 (1H, br s), 7.43 (1H, m), 7.25 (2H, m), 7.17 (1H, m), 4.17 (2H, s), 3.88 (2H, br t, J=8 Hz), 1.58 (2H, m), 1.39-1.29 (2H, m), 0.90 (3H, t, J=7.2 Hz);

¹³C NMR (CDCl₃) δ 167.7, 150.5, 133.8, 131.7, 130.4, 129.9, 129.2, 127.4, 44.2, 30.1, 29.2, 19.9, 13.6;

MS (ESI) m/z 282 (M+1).

### EXAMPLE 70

### 5-(2-Chlorobenzyl)-4-(3-morpholin-4-yl-propyl)-2,4-dihydro-[1,2,4]triazole-3-thione

Starting from (2-chlorophenyl)acetic acid (221 mg, 1.29 mmol) and 3-(4-morpholino)propyl-3-thiosemicarbazide (311 mg, 1.42 mmol) gave the title compound in 20% yield.

¹H NMR (CDCl₃) δ 11.45 (1H, br s), 7.43 (1H, m), 7.25 (2H, m), 7.13 (1H, m), 4.21 (2H, s), 3.98 (2H, br t, J=7.4 Hz), 3.69 (4H, t, J=4.6 Hz), 2.39 (6H, m), 1.89 (2H, m);

¹³C NMR (CDCl₃) δ 167.9, 150.7, 133.7, 131.9, 130.2, 129.9, 129.2, 127.5, 66.8, 55.4, 53.5, 42.7, 29.2, 24.2;

MS (ESI) m/z 353 (M+1), 351 (M−1).

### EXAMPLE 71

### 5-(2-Chlorobenzyl)-4-(tetrahydrofuran-2-yl-methyl)-2,4-dihydro-[1,2,4]triazole-3-thione

The title compound was prepared according to method E with the exception that dichloromethane was used as the only solvent in the coupling step. Starting from (2-chlorophenyl)acetic acid (270 mg, 1.58 mmol) and 4-(2-tetrahydrofurfuryl)-3-thiosemicarbazide (306 mg, 1.74 mmol) gave the title compound in 64% yield.

¹H NMR (CDCl₃) δ 11.15 (1H, br s), 7.41 (1H, m), 7.24 (2H, m), 7.16 (1H, m), 4.37 (1H, d, J=16.8 Hz), 4.30 (2H, m), 4.22 (1H, d, J=16.8 Hz), 3.91 (1H, m), 3.79-3.68 (2H, m), 2.12 (1H, app sextet, J=6.6 Hz), 1.92 (2H, m), 1.70-1.60 (1H, m);

¹³C NMR (CDCl₃) δ 167.9, 151.7, 133.9, 132.3, 130.6, 129.8, 129.0, 127.3, 76.5, 68.2, 48.3, 29.6, 29.0, 25.8;

MS (ESI) m/z 310 (M+1), 308 (M−1).

### EXAMPLE 72

### 5-(2-Chlorobenzyl)-4-(4-chlorophenyl)-2,4-dihydro-[1,2,4]triazole-3-thione

The title compound was prepared according to method E, with the exception that in the second step acetone was added to dissolve the intermediate. Starting from (2-chlorophenyl)acetic acid (214 mg, 1.25 mmol) and 4-(4-chlorophenyl)-3-thiosemicarbazide (278 mg, 1.38 mmol) gave the title compound in 77% yield.

¹H NMR (CD₃OD) δ 7.41 (2H, m), 7.25 (1H, m), 7.23-7.12 (2H, m), 7.07 (3H, m), 3.96 (2H, s);

¹³C NMR (CDCl₃) δ 168.1, 150.3, 135.8, 133.6, 131.4, 131.3, 130.3, 129.6, 129.2, 129.0, 128.7, 126.7, 29.4;

MS (ESI) m/z 336 (M+1).

### EXAMPLE 73

### 5-(1H-Indol-3-ylmethyl)-4-(2-methoxyphenyl)-2,4-dihydro-[1,2,4]triazole-3-thione

The title compound was prepared according to method D with the exception that the cyclization using NaOH was performed at ambient temperature. Indole-3-acetic acid (0.15 g, 0.86 mmol) and 4-(2-methoxyphenyl)-3-thiosemicarbazide (0.19 g, 0.94 mmol) were used and this afforded 0.13 g (43%) of the product.

¹H NMR (DMSO-d₆) δ 13.6 (1H, br s), 10.8 (1H, s), 7.49 (1H, m), 7.29 (2H, m), 7.16 (1H, d, J=8.4 Hz), 7.09 (1H, m), 7.03 (2H, m), 6.93 (1H, m), 6.67 (1H, d, J=2.2 Hz), 3.87 (1H, d, J=16 Hz), 3.78 (1H, d, J=16 Hz), 3.58 (3H, s);

¹³C NMR (DMSO-d₆) δ 168.4, 154.9, 152.3, 136.4, 131.6, 130.4, 127.0, 124.1, 122.3, 121.4, 120.9, 118.8, 118.4, 112.9, 111.7, 107.1, 55.9, 22.4;

MS (ESI) m/z 337 (M+1).

### EXAMPLE 74

### 5-(1H-Indol-3-ylmethyl)-4-(2-methylphenyl)-2,4-dihydro-[1,2,4]triazole-3-thione

The title compound was prepared according to method D with the exception that the cyclization using NaOH was performed at ambient temperature. Starting from indole-3-acetic acid (0.15 g, 0.86 mmol) and 4-(2-methylphenyl)-3-thiosemicarbazide (0.17 g, 0.94 mmol) afforded 0.18 g (66%) of the product.

¹H NMR (DMSO-d₆) δ 13.7 (1H, br s), 10.8 (1H, s), 7.40 (1H, m), 7.32 (3H, m), 7.24 (1H, d, J=7.3 Hz), 7.18 (1H, d, J=7.4 Hz), 7.04 (1H, t, J=7.4 Hz), 6.91 (1H, t, J=7.3 Hz), 6.48 (1H, d, J=2.1 Hz), 3.91 (1H, d, J=16 Hz), 3.80 (1H, d, J=16 Hz), 1.53 (3H, s); ¹³C NMR (DMSO-d₆) δ 167.2, 151.4, 136.6, 136.0, 132.6, 130.7, 129.8, 128.4, 126.9, 126.6, 123.7, 121.1, 118.6, 118.0, 111.4, 106.2, 22.2, 16.7;

MS (ESI) m/z 321 (M+1).

### EXAMPLE 75

### 5-Cyclopentylmethyl-4-phenyl-2,4-dihydro-[1,2,4]triazole-3-thione

A solution of cyclopentylacetic acid (0.26 g, 2.0 mmol) in SOCl₂ (0.4 mL) was stirred at ambient temperature for 1 h. The excess of SOCl₂ was evaporated in vacuo. The residue was dissolved in chloroform (5 mL). 4-Phenyl-3-thiosemicarbazide (0.32 g, 1.9 mmol) and pyridine (0.1 mL) were added and the resulting solution was stirred for 1.5 h. The solvent was evaporated in vacuo and the resulting oil was dissolved in MeOH (1 mL) and 1% NaOH (5 mL) was added. The reaction mixture was stirred at ambient temperature overnight and then at 50° C. for 2 h. The reaction mixture was diluted with water and neutralized with 2M hydrochloric acid. The resulting precipitate was collected by filtration and washed with water giving 0.16 g (31%) of the title compound.

¹H NMR (DMSO-d₆) δ 13.7 (1H, s), 7.54 (3H, m), 7.39 (2H, m), 2.40 (2H, d, J=7.0 Hz), 1.95 (1H, d, J=7.0 Hz), 1.63 (2H, m), 1.52-1.37 (4H, m), 1.04 (2H, m);

¹³C NMR (DMSO-d₆) δ 167.5, 151.8, 133.8, 129.4, 128.3, 36.3, 31.7, 31.1, 24.4;

MS (ESI) m/z 260 (M+1).

### EXAMPLE 76

### 5-(2-Chlorobenzyl)-4-(2-chlorophenyl)-2,4-dihydro-[1,2,4]triazole-3-thione

The title compound was prepared the same way as Example 75 starting from (2-chlorophenyl)acetic acid (0.25 g, 1.5 mmol) and 4-(2-chlorophenyl)-3-thiosemicarbazide (0.28 g, 1.4 mmol). Furthermore, in the cyclization reaction, 2% NaOH (10 mL) was used and the total reaction time was 2.5 h at 50° C. Yield: 59 mg (13%) of the title compound.

¹H NMR (DMSO-d₆) δ 13.8 (1H, s), 7.64 (1H, dd, J=8.1 Hz, 1.3 Hz), 7.55 (1H, dt, J=7.6 Hz, 1.5 Hz), 7.48 (1H, dt, J=7.8Hz, 1.3 Hz), 7.40 (1H, dd, J=7.8 Hz, 1.7 Hz), 7.34 (1H, dd, J=7.8 Hz, 1.3 Hz), 7.25 (1H, dt, J=7.3 Hz, 1.8 Hz), 7.20 (1H, dt, J=7.6 Hz, 1.2 Hz), 7.13 (1H, dd, J=7.6 Hz, 1.6 Hz), 3.91 (1H, d, J=16.4 Hz), 3.84 (1H, d, J=16.4 Hz);

¹³C NMR (DMSO-d₆) δ 168.0, 149.9, 133.3, 132.0, 131.8, 131.7, 131.5, 131.0, 130.8, 130.3, 129.2, 129.1, 128.4, 127.2, 29.4;

MS (ESI) m/z 336 (M+1).

### EXAMPLE 77

### 5-[(2-Chlorophenyl)hydroxymethyl]-4-(4-nitrophenyl)-2,4dihydro-[1,2,4]triazole-3-thione

The title compound was prepared according to method E in 65% overall yield starting from (2-chlorophenyl)hydroxyacetic acid (132 mg, 0.71 mmol) and 4-(4-nitrophenyl)-3-thiosemicarbazide (151 mg, 0.71 mmol).

¹H NMR (CD₃OD) δ 8.35 (2H, br d), 7.58 (2H, br d), 7.51 (1H, m), 7.32-7.21 (3H, m), 5.86 (1H, s);

¹³C NMR (CD₃OD) δ 170.6, 154.0, 149.9, 140.7, 137.7, 133.3, 131.4, 130.8, 130.4, 129.7, 128.2, 125.7, 65.8;

MS (ESI) m/z 363 (M+1).

The compounds of Examples 78 to 91 were obtained from Menai Organics Ltd, Menai Technology Centre, Deiniol Road, Bangor, Gwynedd, N. Wales, LL57 2UP, UK.

### EXAMPLE 78

### 5-(2,3-Dichlorophenoxymethyl)-4-phenyl-2,4dihydro-[1,2,4]triazole-3-thione

MS (ESI) m/z 352 and 354 (M+1).

### EXAMPLE 79

### 5-(4-Chloro-2-methylohenoxymethyl)-4-phenyl-2,4-dihydro-[1,2,4]triazole-3-thione

MS (ESI) m/z 332 (M+1).

### EXAMPLE 80

### 5-(2-Chlorobenzyl)-4-phenyl-2,4-dihydro-[1,2,4]triazole-3-thione

¹H NMR (DMSO-d₆) δ 13.77 (1H, s), 7.48-7.53 (3H, m), 7.36 (1H, dd, J=7.7, 1.5 Hz), 7.32-7.33 (2H, m), 7.22-7.27 (2H, m), 7.19 (1H, dd, J=7.4, 2.3 Hz), 3.93 (2H, s);

¹³C NMR (DMSO-d₆) δ 167.9, 150.1, 133.5, 133.1, 132.4, 131.3, 129.4, 129.3, 129.1, 128.9, 128.1, 127.2, 29.5;

MS (ESI) m/z 300.1 and 302.1 (M−1).

### EXAMPLE 81

### 5-(4-Bromophenoxymethyl)-4-phenyl-2,4-dihydro[1,2,4]triazole-3-thione

MS (ESI) m/z 362 and 364 (M+1).

### EXAMPLE 82

### 5-(1H-Indol-3-ylmethyl)-4-phenyl-2,4-dihydro-[1,2,4]triazole-3-thione

MS (ESI) m/z 307.2 (M+1).

### EXAMPLE 83

### 5-(3-Chlorobenzyl)-4-phenyl-2,4-dihydro-[1,2,4]triazole-3-thione

MS (ESI) m/z 302 (M+1).

### EXAMPLE 84

### 5-(6-Bromonaphthalen-2-yloxylmethyl)-4-phenyl-2,4-dihydro-[1,2,4]triazole-3-thione

MS (ESI) m/z 412 and 414 (M+1).

### EXAMPLE 85

### 5-(4-Methoxyphenoxymethyl)-4-phenyl-2,4-dihydro-[1,2,4]triazole-3-thione

MS (ESI) m/z 314 (M+1).

### EXAMPLE 86

### 5-(3,4-Dimethoxybenzyl)-4phenyl-2,4-dihydro-[1,2,4]triazole-3-thione

MS (ESI) m/z 328.2 (M+1).

### EXAMPLE 87

### 5-(3-Methoxyphenyl)-4-phenyl-2,4-dihydro-[1,2,4]triazole-3-thione

MS (ESI) m/z 284 (M+1).

### EXAMPLE 88

### 5-(3-Dimethylaminophenoxymethyl)-4-phenyl-2,4-dihydro-[1,2,4]triazole-3-thione

MS (ESI) m/z 327 (M+1).

### EXAMPLE 89

### 4-Phenyl-5-thiophen-2-yl-2,4-dihydro-[1,2,4]triazole-3-thione

MS (ESI) m/z 260 (M+1).

### EXAMPLE 90

### 5-(4-Hydroxyphenyl)-4-phenyl-2,4-dihydro-[1,2,4]triazole-3-thione

MS (ESI) m/z 270 (M+1).

### EXAMPLE 91

### 5-(4-Carboxyphenoxy)methyl-4-(4-chlorophenyl)-2,4-dihydro-[1,2,4]triazole-3-thione

MS (ESI) m/z 362 (M+1).

The compounds of Examples 92 to 96 were obtained from Maybridge Chemical Company Ltd., Trevillet, Tintangel, Cornwall PL34 OHW, UK.

### EXAMPLE 92

### 5-(Hydroxyphenylmethyl)-4-phenyl-2,4-dihydro-[1,2,4]triazole-3-thione

MS (ESI) m/z 282.2 (M−1).

### EXAMPLE 93

### 5-Benzyl-4-phenyl-1,2-dihydro-[1,2,4]triazol-3-thione

MS (ESI) m/z 268 (M+1).

### EXAMPLE 94

### 4-(3-Chlorophenyl)-5-(5-methyl-2-nitrophenyl)-2,4-dihydro-[1,2,4]triazole-3-thione

MS (ESI) m/z 347 (M+1).

### EXAMPLE 95

### 4-Phenyl-5-(4-trifluoromethoxyphenoxymethyl)-2,4-dihydro-[1,2,4]triazole-3-thione

MS (ESI) m/z 368 (M+1).

### EXAMPLE 96

### 4-Phenyl-5-(4-trifluoromethylsulfanyl-phenoxymethyl)-2,4-dihydro-[1,2,4]triazole-3-thione

MS (ESI) m/z 384 (M+1).

### EXAMPLE 97

### 5-(4-Cyclohexylphenoxvmethyl)-4-phenyl-2,4-dihydro-[1,2,4]triazole-3-thione

Obtained from SPECS, SPECS and BioSPECS B. V., Fleminglaan 16, 2289 CP Rijswijk, The Netherlands.

MS (ESI) m/z 366 (M+1).

### EXAMPLE 98

### 4-Phenyl-5-phenylamino-2,4-dihydro-[1,2,4]triazole-3-thione Obtained from Sigma-Aldrich (Salor)

MS (ESI) m/z 269.1 (M+1), 267.2 (M−1).

### EXAMPLE 99

### 4-Phenyl-5-thiophen-2-ylmethyl-2,4-dihydro-[1,2,4]triazole-3-thione Obtained from Ambinter, 46 quai Louis Blériot, Paris F-75016, France

MS (ESI) m/z 274.0 (M+1), 272.0 (M−1).

Except where otherwise indicated, the compounds of Examples 100 to 127 were prepared using the procedure of General Method D.

### EXAMPLE 100

### 5-(2-Methoxybenzyl)-4-phenyl-2,4-dihydro-[1,2,4]triazole-3-thione

The title compound was obtained in 57% yield starting from (2-methoxyphenyl)acetic acid (199 mg, 1.2 mmol) and 4-phenyl-3-thiosemicarbazide (200 mg, 1.2 mmol).

¹H NMR (DMSO-d₆) δ 13.70 (1H, s), 7.49 (3H, m), 7.26 (2H, m), 7.19 (1H, m), 6.97 (1H, dd, J=7.6 Hz, 1.5 Hz), 6.83 (2H, m), 3.75 (2H, s), 3.59 (3H, s);

¹³C NMR (DMSO-d₆) δ 167.6, 156.6, 151.2, 133.6, 129.9, 129.3, 129.2, 128.4, 128.2, 122.7, 120.2, 110.7, 55.3, 25.8;

MS (ESI) m/z 298 (M+1).

### EXAMPLE 101

### 5-(2-Butoxybenzyl)-4-phenyl-2,4-dihydro-[1,2,4]triazole-3-thione

Starting with (2-butoxyphenyl)acetic acid (124.5 mg, 598 μmol) and 4-phenyl-3-thiosemicarbazide (100 mg, 598 μmol) afforded 43 mg (21%) of the title compound.

¹H NMR (DMSO-d₆) δ 13.68 (1H, s), 7.48 (3H, m), 7.25 (2H, m), 7.17 (1H, m), 6.97 (1H, d, J=7.6 Hz), 6.85 (1H, d, J=8.1 Hz), 6.79 (1H, t, J=7.4 Hz), 3.80 (2H, t, J=6.3 Hz), 3.76 (2H, s), 1.53 (2H, m), 1.30-1.21 (2H, m), 0.87 (3H, t, J=7.4 Hz);

¹³C NMR (DMSO-d₆) δ 167.6, 156.1, 151.2, 133.7, 130.1, 129.2, 129.2, 128.4, 128.0, 122.7, 120.0, 111.4, 67.1, 30.6, 26.3, 18.6, 13.6;

MS (ESI) m/z 340 (M+1).

### EXAMPLE 102

### 5-(3-Butoxybenzyl)-4-phenyl-2,4-dihydro-[1,2,4]triazole-3-thione

### a) (3-Butoxyphenyl)acetic acid

n-Iodobutane (1.13 mL, 9.86 mmol) in DMSO (10 mL) was added dropwise to (3-hydroxyphenyl)acetic acid (1.5 g, 9.86 mmol) in 10% NaOH (aq) (7.9 mL) and DMSO (3 mL) at 80° C. and the reaction mixture was then stirred at that temperature for 3.5 h. After cooling, the reaction mixture was poured into 1M HCl (200 mL). The precipitate was washed with water and n-hexane sequentially. The mother liquor and water were extracted with Et₂O (3x). The combined organic phases were washed with brine, dried (MgSO4), filtered and concentrated in vacuo. The residue was recrystallized from n-hexane. The n-hexane used to wash the precipitate yielded a second crop of solid upon partial concentration. Total yield 700 mg (34%) of the title compound.

¹H NMR (DMSO-d₆) δ 12.28 (1H, s), 7.20 (1H, t, J=7.8 Hz), 6.80 (3H, m), 3.94 (2H, t, J=6.3 Hz), 3.52 (2H, s), 1.69 (2H, m), 1.48-1.38 (2H, m), 0.93 (3H, t, J=7.3 Hz);

¹³C NMR (DMSO-d₆) δ 172.5, 158.6, 136.4, 129.2, 121.4, 115.6, 112.5, 67.0, 40.7, 30.8, 18.7, 13.7;

MS (ESI) m/z 207 (M−1).

### b) 5-(3-Butoxybenzyl)-4phenyl-2,4-dihydro-[1,2,4]triazole-3-thione

Starting with (3-butoxyphenyl)acetic acid (124.5 mg, 598 μmol) and 4-phenyl-3-thiosemicarbazide (100 mg, 598 μmol) afforded 107 mg (53%) of the title compound.

¹H NMR (DMSO-d₆) δ 13.78 (1H, s), 7.47 (3H, m), 7.22 (2H, m), 7.09 (1H, m), 6.73 (1H, d, J=8.1 Hz), 6.48 (1H, d, J=7.5 Hz), 6.42 (1H, s), 3.80 (4H, m), 1.63 (2H, m), 1.40 (2H, m), 0.92 (3H, t, J=7.4 Hz);

¹³C NMR (DMSO-d₆) δ 167.9, 158.6, 151.1, 135.9, 133.6, 129.3, 129.2, 128.3, 120.6, 114.7, 113.0, 66.9, 31.4, 30.7, 18.7, 13.7;

MS (ESI) m/z 340 (M+1).

### EXAMPLE 103

### 5-Naphthalen-1-ylmethyl-4-phenyl-2,4dihydro-[1,2,4]triazole-3-thione

(Kothari, P. J. et al. J. Heterocyclic Chem. 1978, 15, 1101-1104. Suman, S. P. et al. J. Indian Chem. Soc. 1980, 57, 420-422).

Starting with (1-napthyl)acetic acid (111 mg, 598 μmol) and 4-phenyl-3-thiosemicarbazide (100 mg, 598 μmol) afforded 152 mg (80%) of the title compound.

¹H NMR (DMSO-d₆) δ 13.75 (1H, s), 7.91 (2H, m), 7.79 (1H, d, J=8.1 Hz), 7.49 (5H, m), 7.32 (3H, m), 6.99 (1H, d, J=6.6 Hz), 4.30 (2H, s);

¹³C NMR (DMSO-d₆) δ 167.9, 151.0, 133.6, 133.2, 131.3, 130.4, 129.4, 129.3, 128.4, 128.3, 127.7, 127.3, 126.2, 125.7, 125.2, 123.7, 29.2;

MS (ESI) m/z 318 (M+1).

### EXAMPLE 104

### 5-(2-Chloro-5-methoxybenzyl)-4-phenyl-2,4-dihydro-[1,2,4]triazole-3-thione

### a) (2-Chloro-5-methoxybenzyl)acetic acid

NCS (884 mg, 6.6 mmol) in dry DMF (4.4 mL) was added dropwise to (3-methoxyphenyl)acetic acid (1 g, 6.0 mmol) in dry DMF (4 mL) at 0° C. The reaction mixture was stirred at ambient temperature for 24 h, poured into water, extracted with CHCl₃, dried over MgSO₄, filtered and concentrated in vacuo. Purification by flash chromatography on silica gel (hexane/EtOAc) yielded 813 mg (67%) of the title compound.

¹H NMR (DMSO-d₆) δ 12.42 (1H, s), 7.33 (1H, d, J=8.6 Hz), 7.00 (1H, d, J=3.1 Hz), 6.87 (1H, dd, J=8.9, 3.3 Hz), 3.74 (3H, s), 3.67 (2H, s);

¹³C NMR (DMSO-d₆) δ 171.3, 157.9, 134.2, 129.6, 124.9, 117.7, 114.0, 55.4, 38.8;

MS (EI) m/z 200 (M+).

### b) 5-(2-Chloro-5-methoxybenzyl)-4-phenyl-2,4-dihydro-[1,2,4]triazole-3-thione

Starting with (2-chloro-5-methoxyphenyl)acetic acid (120 mg, 598 μmol) and 4-phenyl-3-thiosemicarbazide (100 mg, 598 μmol) afforded 49 mg (25%) of the title compound.

¹HNMR (DMSO-d₆) δ 13.77 (1H, s), 7.54 (3H, m), 7.33 (2H, m), 7.26 (1H, d, J=9.1 Hz), 6.84 (1 H, dd, J=8.9, 2.9 Hz), 6.74 (1H, d, J=2.5 Hz), 3.88 (2H, s), 3.69 (3H, s);

¹³C NMR (DMSO-d₆) δ 167.8, 157.9, 150.0, 133.5, 133.3, 129.8, 129.5, 129.4, 128.2, 124.4, 117.0, 114.4, 55.4, 29.9;

MS (ESI) m/z 332 (M+1).

### EXAMPLE 105

### 5-(3-Chlorobenzyl)-4-o-tolyl-2,4-dihydro-[1,2,4]triazole-3-thione

The title compound was prepared according to the general method of Example D with the exception that the title compound was further purified by dissolving in methanol (10 mL) and 2% NaOH (5 mL) and precipitated with 1M HCl. Starting with (3-chlorophenyl)-acetic acid (0.34 g, 2.0 mmol) and 4-(2-methylphenyl)-3-thiosemicarbazide (0.36 g, 2.0 mmol) afforded 0.46 g (73%) of the title compound.

¹H NMR (DMSO-d₆) δ 13.6 (1H, br s), 6.93-7.24 (6H, m), 6.53-6.67 (2H, m), 3.52 (2H, m), 1.48 (3H, s).

MS (ESI) m/z 316 (M+1).

### EXAMPLE 106

### 5-(2-Chloro-6-fluoro-3-methylbenzyl)-4-phenyl-2,4-dihydro-[1,2,4]triazole-3-thione

Starting with (2-chloro-6-fluoro-3-methylphenyl)acetic acid (121 mg, 598 μmol) and 4-phenyl-3-thiosemicarbazide (100 mg, 598 μmol) afforded 96 mg (48%) of the title compound.

¹H NMR (DMSO-d₆) δ 13.74 (1H, s), 7.55 (3H, m), 7.43 (2H, m), 7.31 (1H, dd, J=8.4, 6.4 Hz), 7.09 (1H, t, J=8.8 Hz), 3.91 (2H, s), 2.26 (3H, s);

¹³C NMR (DMSO-d₆) δ 167.8, 159.0 (d, J=245.3 Hz), 149.3, 134.33 (d, J=5.4 Hz), 133.4, 132.0 (d, J=3.8 Hz), 130.6 (d, J=9.2 Hz), 129.6, 129.5, 128.1, 120.6 (d, J=17.6 Hz), 113.6 (d, J=22.3 Hz), 23.9 (d, J=3.8 Hz), 19.6;

MS (ESI) m/z 334 (M+1).

### EXAMPLE 107

### 5-(Biphenyl-2-ylmethyl)-4-phenyl-2,4-dihydro-[1,2,4]triazole-3-thione

Starting with (biphenyl-2-yl)acetic acid (127 mg, 598 μmol) and 4-phenyl-3-thiosemicarbazide (100 mg, 598 μmol) afforded 121 mg (59%) of the title compound.

¹HNMR (DMSO-d₆) δ 13.70 (1H, s), 7.45-7.27 (8H, m), 7.21 (1H, m), 7.11 (1H, m), 6.97 (4H, m), 3.73 (2H, s);

¹³C NMR (DMSO-d₆) δ 167.7, 151.3, 141.5, 140.1, 133.3, 131.9, 129.6, 129.6 129.2, 129.2, 128.6, 128.1, 128.0, 127.4, 127.1, 127.1, 29.4;

MS (ESI) m/z 344 (M+1).

### EXAMPLE 108

### 5-(3-Oxo-indan-1-yl)-4-phenyl-2,4-dihydro-[1,2,4]triazole-3-thione

Starting with 3-oxo-indan-1-carboxylic acid (105 mg, 598 μmol) and 4-phenyl-3-thiosemicarbazide (100 mg, 598 μmol) afforded 96 mg (52%) of the title compound.

¹H NMR (DMSO-d₆) δ 13.80 (1H, s), 7.70 (1H, t, J=7.6 Hz), 7.58 (2H, m), 7.48 (4H, m), 7.40 (2H, br s), 4.63 (1H, dd, J=7.3, 4.8 Hz), 2.82-2.70 (2H, m);

¹³C NMR (DMSO-d₆) δ 202.9, 168.4, 153.1, 152.0, 136.3, 134.9, 133.5, 129.5, 129.4, 128.8, 128.6, 127.4, 122.8, 41.7, 35.2;

MS (ESI) m/z 308 (M+1).

### EXAMPLE 109

### 5-(4Chlorophenoxy)methyl-4-phenyl-2,4-dihydro-[1,2,4]triazole-3-thione

(Demirayak, S. et al. Acta Pharm. Turcica 1990, 32, 35-40).

Starting from (4-chlorophenoxy)acetic acid (560 mg, 3 mmol) and 4-phenylthiosemicarbazide (501 mg, 3 mmol), afforded 834 mg (88%) of the title compound.

¹H NMR (300 MHz, DMSO-d₆) δ 14.07 (1H, s), 7.52-7.42 (5H, m), 7.28 (2H, m), 6.84 (2H, m), 4.97 (2H, s);

MS (ESI) m/z 318 (M+1).

### EXAMPLE 110

### 5-(4-Acetylphenoxy)methyl-4-phenyl-2,4-dihydro-[1,2,4]triazole-3-thione

Starting from (4-acetylphenoxy)acetic acid (673 mg, 3.47 mmol) and 4-phenylthiosemicarbazide (580 mg, 3.47 mmol) afforded 821 mg (72%) of the title compound.

¹H NMR (300 MHz, DMSO-d₆) δ 14.06 (1H, s), 7.85 (2H, d, J=8.5 Hz), 7.48 (5H, m), 6.94 (2H, d, J=8.5 Hz), 5.08 (2H, s), 3.30 (3H, s);

MS (ESI) m/z 326 (M+1).

### EXAMPLE 111

### 5-(3-Methoxyphenoxy)methyl-4-phenyl-2,4-dihydro-[1,2,4]triazole-3-thione

Starting from (3-methoxyphenoxy)acetic acid (700 mg, 3.84 mmol) and 4-phenylthiosemicarbazide (643 mg, 3.84 mmol) afforded 464 mg (39%) of the title compound.

¹H NMR (300 MHz, DMSO-d₆) δ 14.05 (1H, s), 7.53-7.43 (5H, m), 7.11 (1H, t, J=8.1 Hz), 6.51 (1H, d, J=8.3 Hz), 6.39 (2H, m), 4.95 (2H, s), 3.68 (3H, s);

MS (ESI) m/z 314 (M+1).

### EXAMPLE 112

### 5-(2-Methoxyphenoxy)methyl-4-phenyl-2,4-dihydro-[1,2,4]triazole-3-thione

(Demirayak, S. et al. Acta Pharm. Turcica 1990, 32, 35-40).

Starting from (2-methoxy-phenoxy)acetic acid (200 mg, 1.1 mmol) and 4-phenylthiosemicarbazide (210 mg, 1.1 mmol) afforded 101 mg (29%) of the title compound.

¹H NMR (300 MHz, CDCl₃) δ 11.72 (1H, s), 7.52 (5H, m), 7.00 (1H, t, J=7.2 Hz), 6.88-6.75 (3H, m), 4.91 (2H, s), 3.80 (3H, s);

MS (ESI) m/z 314 (M+1).

### EXAMPLE 113

### 5-Phenoxymethyl-4-phenyl-2,4-dihydro-[1,2,4]triazole-3-thione

(Demirayak, S. et al. Acta Pharm. Turcica 1990, 32, 35-40).

Starting from phenoxyacetic acid (200 mg, 1.3 mmol) and 4-phenylthiosemicarbazide (220 mg, 1.3 mmol) afforded 99 mg (35%) of the title compound.

¹H NMR (300 MHz, DMSO-d₆) δ 14.05 (1H, s), 7.53-7.43 (5H, m), 7.23 (2H, t, J=7.2 Hz), 6.94 (1H, t, J=7.5 Hz), 6.81 (2H, d, J=7.8 HI), 4.96 (2H, s);

MS (ESI) m/z 284 (M+1).

### EXAMPLE 114

### 5-(4-Butoxyphenoxy)methyl-4-phenyl-2,4-dihydro-[1,2,4]triazole-3-thione

Starting from (4-butoxyphenoxy)acetic acid (294 mg, 1.3 mmol) and 4-phenylthiosemicarbazide (220 mg, 1.3 mmol) afforded 221 mg (47%) of the title compound.

¹H NMR (300 MHz, CDCl₃) δ 11.8 (1H, s), 7.53 (3H, m), 7.43 (2H, m), 6.78-6.68 (4H, m), 4.83 (2H, s), 3.88 (2H, t, J=6.6 Hz), 1.73 (2H, m), 1.53-1.43 (2H, m), 0.96 (3H, t, J=7.2 Hz);

MS (ESI) m/z 356 (M+1).

### EXAMPLE 115

### 5-(2-Chlorophenoxy)methyl-4-phenyl-2,4-dihydro-[1,2,4]triazole-3-thione

(Turan-Zitouni, G. et al. Farmaco 2002, 57, 573-575. Bahel, S. C. et al. J. Indian. Chem. Soc. 1982, 59, 1127-1129. Pathak, R. B. et al. Bokin Bobai 1980, δ, 149-153).

Starting from (2-chlorophenoxy)acetic acid (200 mg, 1.1 mmol) and 4-phenylthiosemicarbazide (179 mg, 1.1 mmol) afforded 146 mg (46%) of the title compound.

¹H NMR (300 MHz, DMSO-d₆) δ 14.10 (1H, s), 7.48 (5H, m), 7.38 (1H, d, J32 7.8 Hz), 7.24 (1H, t, J=7.8 Hz), 7.10 (1H, d, J=8.1 Hz), 6.96 (1H, t, J=7.6 Hz), 5.05 (2H, s);

MS (ESI) m/z 318 (M+1).

### EXAMPLE 116

### 5-(3-Chlorophenoxy)methyl-4-phenyl-2,4-dihydro-[1,2,4]triazole-3-thione

Starting from (3-chlorophenoxy)acetic acid (200 mg, 1.1 mmol) and 4-phenylthiosemicarbazide (179 mg, 1.1 mmol) afforded 162 mg (51%) of the title compound.

¹H NMR (300 MHz, DMSO-d₆) δ 14.06 (1H, s), 7.53-7.43 (5H, m), 7.25 (1H, t, J=8.1 Hz), 7.00 (1H, d, J=8.1 Hz), 6.93 (1H, s), 6.79 (1H, d, J=8.3 Hz), 5.01 (2H, s);

MS (ESI) m/z 318 (M+1).

### EXAMPLE 117

### 5-(2-Methylcarbamoylphenoxy)methyl-4-phenyl-2,4-dihydro-[1,2,4]triazole-3-thione

Starting from (2-methylcarbamoylphenoxy)acetic acid (200 mg, 0.96 mmol) and 4-phenylthiosemicarbazide (160 mg, 0.96 mmol) afforded 25 mg (8%) of the title compound.

¹H NMR (300 MHz, DMSO-d₆) δ 14.10 (1H, s), 7.88 (1H, br s), 7.55 (1H, d, J=7.6 Hz), 7.49 (5H, m), 7.38 (1H, m), 7.07 (2H, m), 5.10 (2H, s), 2.72 (3H, d, J=5.1 Hz),

MS (ESI) m/z 341 (M+1).

### EXAMPLE 118

### 5-(3-Butoxy-phenoxymethyl)-4-phenyl-2,4-dihydro-[1,2,4]triazole-3-thione

Starting from 3-butoxyphenoxyacetic acid (200 mg, 0.89 mmol) and phenylacetic acid hydrazide (149 mg, 0.89 mmol) afforded 55 mg (17%) of the title compound.

¹H NMR (DMSO-d₆): δ 7.53 (3H, m), 7.44 (2H, m), 7.10 (1H, t, J=8.1 Hz), 6.50 (1H, m), 6.37 (2H, m), 4.94 (2H, s), 3.88 (2H, t, J=6.4 Hz), 1.66 (2H, m), 1.46-1.36 (2H, m), 0.92 (3H, t, J=7.4 Hz);

¹³C NMR (DMSO-d₆): δ 168.6, 159.3, 158.3, 148.0, 133.4, 129.9, 129.5, 129.2, 128.0, 107.7, 107.0, 101.6, 67.1, 60.1, 30.6, 18.7, 13.7;

MS (ESI) m/z 356 (M+1).

### EXAMPLE 119

### 5-Isochroman-1-yl-4-phenyl-2,4-dihydro-[1,2,4]triazole-3-thione

Starting with isochroman-1-carboxylic acid (107 mg, 598 μmol, obtained from Rare Chemicals) and 4-phenyl-3-thiosemicarbazide (100 mg, 598 μmol) afforded 97 mg (53%) of the title compound.

¹H NMR (DMSO-d₆) δ 13.96 (1H, s), 7.43 (3H, m), 7.17 (4H, m), 7.03 (2H, d, J=6.6 Hz), 5.77 (1H, s), 3.84 (1H, m), 3.71 (1H, m), 2.58 (1H, m), 2.33 (1H, m);

¹³C NMR (DMSO-d₆) δ 169.1, 151.31, 133.9, 133.6, 131.5, 129.1, 128.7, 128.6, 127.3, 126.0, 125.9, 69.1, 61.8, 27.0;

MS (ESI) m/z 310 (M+1).

### EXAMPLE 120

### 5-{3-[(Methylamino)carbonyl]benzyl}-4-phenyl-2,4-dihydro-[1,2,4]triazole-3-thione

### a) 3-(Cyanomethyl)benzoic acid

Lithium hydroxide (493 mg, 11.7 mmol) was added to a stirred solution of methyl 3-(cyanomethyl)benzoate (1.029 g, 5.87 mmol) in THF (10 ml) and water (0.5 ml) at ambient temperature. The reaction mixture was stirred at 50° C. overnight. The solvent was evaporated and the residue partitioned between water and diethyl ether. The water phase was extracted 3 times with diethyl ether. The aqueous phase was acidified with conc. HCl and was extracted an additional 3 times with diethyl ether. The collected organic phases were dried (Na₂SO₄) and concentrated in vacuo, giving 745 mg (79%) of the title compound.

¹H NMR (DMSO-d₆): ι 8 13.11 (1H, s), 7.95 (1H, s), 7.90 (1H, d, J=7.6 Hz), 7.60 (1H, m), 7.53 (1H, t, J=7.6 Hz), 4.14 (2H, s);

MS (ESI) m/z 160 (M−1).

### b) 3-(Cyanomethyl)-N-methylbenzamide

Thionyl chloride (0.4 ml, 5.55 mmol) was added dropwise to a stirred and cooled (0° C.) solution of 3-(cyanomethyl)benzoic acid (745 mg, 4.62 mmol) and DM (0.3 ml) in anhydrous dichloromethane (5 ml). The resulting mixture was refluxed for 1.5 h. The reaction mixture was then cooled to ambient temperature and added dropwise to methylamine (1.43 ml, 16.6 mmol, 40% solution in water) at 0° C., and the resulting mixture was stirred at 0° C. for 1 h. The reaction mixture was then partitioned between water and dichloromethane, and the water phase was extracted with dichloromethane. The combined organic phases were dried (Na₂SO₄) and concentrated in vacuo to give 778 mg (97%) of the title compound.

¹H NMR (DMSO-d₆): δ 8.49 (1H, br s), 7.83 (1H, s), 7.78 (1H, m), 7.49 (2H, m), 4.10 (2H, s), 2.79 (3H, d, J=4.55 Hz);

MS (ESI) m/z 175 (M+1).

### c) {3-[(Methylamino)carbonyl]phenyl}acetic acid

3-(Cyanomethyl)-N-methylbenzamide (778 mg, 4.47 mmol) in 6 M HCl (50 ml) was stirred under reflux for 4 h. The solution was concentrated and the crude product was dissolved in diethyl ether and washed with water and brine. The organic phase was dried (Na₂SO₄) and concentrated in vacuo to afford 95 mg (11%) of the title compound.

¹H NMR (DMSO-d₆): δ 12.43 (1H, br s), 8.39 (1H, m), 7.70 (2H, m), 7.39 (2H, m), 3.62 (2H, s), 2.77 (3H, d, J=4.5 Hz);

MS (ESI) m/z 194 (M+1).

### d) 5-{3-[(Methylamino)carbonyl]benzyl}-4-phenyl-2.4-dihydro-[1,2,4]triazole-3-thione

Starting from {3-[(methylamino)carbonyl]phenyl}acetic acid (95 mg, 0.49 mmol) and 4-phenyl-3-thiosemicarbazide (0.33 g, 2.0 mmol) afforded 14 mg (9%) of the title compound.

hu 1H NMR (DMSO-d₆): δ 13.80 (1H, br s), 8.35 (1H, m), 7.63 (1H, d, J=7.8 Hz), 7.46 (4H, m), 7.25 (3H, m), 7.03 (1H, d, J=7.6 Hz), 3.91 (2H, s), 2.75 (3H, d, J=4.5 Hz);

MS (ESI) m/z 325 (M+1).

### EXAMPLE 121

### 5-Naphthalen-2-ylmethyl-4-phenyl-2,4-dihydro-[1,2,4]triazole-3-thione

(Amir, M. et al. Indian J. Heterocyclic Chem. 1998, 8, 107-110).

Starting from 2-naphthylacetic acid (223 mg, 1.20 mmol) and 4-phenyl-3-thiosemicarbazide (200 mg, 1.2 mmol) afforded 73 mg (19%) of the title compound.

¹H NMR DMSO-d₆): δ 13.79 (1H, br s), 7.84 (1H, m), 7.75 (2H, m), 7.46 (5H, m), 7.38 (1H, s), 7.26 (2H, m), 7.14 (1H, dd, J=8.5, 1.4 Hz), 4.03 (2H, s);

¹³C NMR (DMSO-d6): δ 167.9, 151.1, 133.6, 132.8, 132.2, 131.8, 129.4, 129.2, 128.3, 127.9, 127.5, 127.4, 127.1, 126.9, 126.2, 125.8, 31.6;

MS (ESI) m/z 318 (M+1).

### EXAMPLE 122

### 4-Phenyl-5(pyridin-2-ylmethyl)-2,4-dihydro-[1,2,4]triazole-3-thione

Starting from pyridin-2-yl-acetic acid (200 mg, 1.46 mmol) and 4-phenyl-3-thiosemicarbazide (244 mg, 1.46 mmol) in DMF (5 ml) afforded 56 mg (14%) of the title compound.

¹H NMR (DMSO-d6): δ 13.76 (1H, br s), 8.38 (1H, m), 7.57 (1H, td, J=7.6, 1.9 Hz), 7.41 (3H, m), 7.21 (2H, m), 7.17 (1H, m), 6.99 (1H, d, J=7.8 Hz), 4.07 (2H, s);

¹³C NMR (DMSO-d₆):δ 137.8, 154.7, 150.3, 148.9, 136.5, 133.6, 129.2, 129.0, 128.1, 123.2, 122.0, 34.01;

15 MS (ESI) m/z 269 (M+1).

### EXAMPLE 123

### 5-(2,3-Dimethoxybenzyl)4-phenyl-2,4-dihydro-[1,2,4]triazole-3-thione

Starting from 2,3-dimethoxyphenylacetic acid (235 mg, 1.20 mmol) and 4-phenyl-3-thiosemicarbazide (200 mg, 1.20 mmol) afforded 188 mg (48%) of the title compound.

¹H NMR (DMSO-d₆): δ 13.74 (1H, s), 7.50 (3H, m), 7.29 (2H, m), 6.92 (2H, m), 6.56 (1H, dd, J=6.1, 3.0 Hz), 3.77 (2H, s), 3.75 (3H, s), 3.43 (3H, s);

¹³C NMR (DMSO-d₆): δ 167.7, 152.2, 151.1, 146.3, 133.6, 129.4, 129.3, 128.2, 123.7, 121.5, 112.0, 59.5, 55.6, 26.1;

MS (ESI) m/z 328 (M+1).

### EXAMPLE 124

### 4-Phenyl-5-(2,3,4-trimethoxybenzyl)-2,4-dihydro-[1,2,4]-triazole-3-thione

Starting from 2,3,4-trimethoxyphenylacetic acid (270 mg, 1.20 mmol) and 4-phenyl-3-thiosemicarbazide (200 mg, 1.20 mmol) afforded 155 mg (36%) of the title compound.

¹H NMR (DMSO-d₆): δ 13.71 (1H, s), 7.51 (3H, m), 7.27 (2H, m), 6.65 (2H, s), 3.74 (3H, s), 3.71 (2H, s), 3.66 (3H, s), 3.50 (3H, s);

¹³C NMR (DMSO-d₆): δ 167.7, 152.6, 151.4, 150.9, 141.5, 133.6, 129.3, 129.2, 128.2, 124.1, 120.3, 107.6, 60.2, 55.8, 25.8;

MS (ESI) m/z 358 (M+1).

### EXAMPLE 125

### 5-[(2,5-Dimethyl-1,3-thiazol-4-yl)methyl]-4-phenyl-2,4-dihydro-[1,2,4]triazole-3-thione

Starting from (2,5-dimethyl-1,3-thiazolyl)acetic acid (240 mg, 1.40 mmol) and 4-phenyl-3-thiosemicarbazide (234 mg, 1.40 mmol) afforded 244 mg (60%) of the title compound.

¹H NMR (DMSO-d₆): δ 13.74 (1H, s), 7.44 (3H, m), 7.15 (2H, m), 3.86 (2H, s), 2.46 (3H, s), 1.86 (3H, s);

¹³C NMR (DMSO-d₆): δ 167.9, 161.4,,150.3, 143.4, 133.6, 129.2, 129.0, 128.3, 127.9, 25.6,18.4, 10.1;

MS (ESI) m/z 303 (M+1).

### EXAMPLE 126

### 4-Phenyl-5-(2-phenylethyl)-2,4-dihydro-[1,2,4]triazole-3-thione

(Khan, R. H. et al. Indian J. Pharm. Sci. 1987,49,48-51).

Starting from hydrocinnamic acid (180 mg, 1.20 mmol) and 4-phenyl-3-thiosemicarbazide (200 mg, 1.20 mmol) afforded 77 mg (23%) of the title compound.

¹H NMR (DMSO-d₆): δ 13.70 (1H, s), 7.56 (3H, m), 7.36 (2H, m), 7.23 (2H, m), 7.16 (1H), m), 7.05 (2H, d, J=7.1 Hz), 2.79 (2H, m), 2.71 (2H, m);

¹³C NMR (DMSO-d₆): δ 167.6, 151.5, 140.0, 133.6, 129.5, 129.4, 128.3, 128.2, 128.2, 126.2, 31.2, 27.3;

MS (ESI) m/z 282 (M+1).

### EXAMPLE 127

### 5-[(2-Butoxyphenoxy)methyl]-4-phenyl-2,4-dihydro-[1, 2,4]triazole-3-thione

### (a) (2-Butoxyphenoxy)acetic acid

(2-Hydroxyphenoxy)acetic acid (200 mg, 1.19 mmol) and l-bromobutane (0.13 ml, 1.19 mmol) were dissolved in 10% NaOH solution (1.3 mL) and DMSO (3.7 mL) in a 5 ml Smith Synthesizer vial. The resulting reaction mixture was heated in a Smith Synthesizer microwave oven for 75 min at 150° C. The above reaction was repeated three times and the combined reaction mixtures were poured into 1M HCl (10 ml) and extracted four times with diethyl ether, dried over MgSO₄ and evaporated onto silica gel. The product was purified by flash chromatography using a heptane-ethyl acetate gradient containing 1% formic acid. The purified product was dissolved in 2% NaOH solution and then precipitated with 1 M HCl, filtered and dried in vacuo, affording 484 mg (61%) of the title compound.

¹H NMR (DMSO-d₆): δ 6.91 (1H, m), 6.83-6.79 (2H, m), 6.76 (1H, m), 4.32 (2H, s), 3.94 (2H, t, J=6.5 Hz), 1.69 (2H, quintet, J=6.5 Hz), 1.44 (2H, sextet, J=7.50 Hz), 0.93 (3H, t, J=7.38 Hz);

¹³C NMR (DMSO-d₆): δ 170.9, 148.4, 148.1, 120.5, 120.3, 113.5, 113.3. 67.9, 67.0, 30.9, 18.8, 18.7;

MS (ESI) m/z 223 (M−1).

### (b) 5-[(2-butoxyphenoxy)methyl]-4-phenyl-2,4-dihydro-3H-1,2,4-triazole-3-thione

Starting from (2-butoxyphenoxy)acetic acid (268 mg, 1.20 mmol) and 4phenyl-3-thiosemicarbazide (200 mg, 1.20 mmol) afforded 123 mg (29%) of the title compound.

hu 1H NMR (DMSO-d₆): δ 14.00 (1H, s), 7.49 (5H, m), 6.93 (2H, m), 6.79 (2H, m), 4.89 (2H, s), 3.88 (2H, t, J=6.44 Hz), 1.66 (2H, m), 1.40 (2H, m), 0.92 (3H, t, J=7.32 Hz);

¹³C NMR (DMSO-d₆): δ 168.4, 149.3, 148.1, 146.3, 133.2, 129.3, 129.0, 128.0, 122.9, 120.4, 116.4, 113.5, 67.6, 61.3, 30.7, 18.6, 13.6;

MS (ESI) m/z 258 (M+1).

The compounds of Examples 128 and 129 were prepared using the procedure of General Method A.

### EXAMPLE 128

### 4-Phenyl-5-(tetrahydrofuran-2-ylmethyl)-2,4-dihydro-[1,2,4]triazole-3-thione

Starting from tetrahydrofuran-2-carboxylic acid ethyl ester (340 mg, 2.15 mmol, obtained from TCI Europe) and 4-phenyl-3-thiosemicarbazide (300 mg, 1.79 mmol) afforded 33 mg (6%) of the title compound.

¹H NM (DMSO-d₆): δ 13.75 (1H, s), 7.56 (3H, m), 7.40 (2H, m), 3.91 (1H, quintent, J=6.59 Hz), 3.59 (1H, m), 3.51 (1H, m), 2.66-2.54 (2H, m), 1.90 (1H, m), 1.72 (2H, quintet, J=7.22 Hz), 1.51 (1H, m);

¹³C NMR (DMSO-d₆): δ 167.5, 150.0, 129.45, 133.7, 129.4, 128.4, 75.2, 67.0, 31.5, 30.6, 24.9;

MS (ESI) m/z 262 (M+1).

### EXAMPLE 129

### 4-[4-(2,6-Dimethyl-morpholin4-yl)-phenyl]-5-(diphenylmethyl)-2,4-dihydro-[1,2,4]triazole-3-thione

Starting with ethyl (diphenyl)-acetate (117 mg, 417 μmol) and 4-[4-(2,6-dimethyl-morpholin4-yl)-phenyl]-3-thiosemicarbazide (100 mg, 416 μmol), afforded 17 mg (9%) of the title compound.

¹H NMR (CDCl₃) δ 7.28 (6H, m), 7.13 (4H, d, J=7.0 Hz), 6.87 (4H, m), 5.06 (1H, s), 3.79 (2H, m), 3.50 (2H, d, J=11.7 Hz), 2.48 (2H, t, J=11.4 Hz), 1.28 (6H, d, J=6.0 Hz);

¹³C NMR (CDCl₃) δ 169.3 154.7, 151.7, 138.5, 129.0, 128.9, 128.8, 127.7, 123.8, 115.5, 71.6, 53.8, 48.7, 19.2;

MS (ESI) m/z 457 (M+1).

Except where otherwise indicated, the compounds of Examples 130 to 134 were prepared 20 using the procedure of General Method B.

### EXAMPLE 130

### 5-Benzyl4-(2-furylmethyl)-2,4-dihydro-[1,2,4]triazole-3-thione

Starting from 2-furfuryl isothiocyanate (278 mg, 2.0 mmol) and phenylacetic acid hydrazide (200 mg, 1.33 mmol) afforded 37 mg (7%) of the title compound.

¹H NMR (DMSO-d₆): δ 13.66 (1H, br s), 7.59 (1H, s), 7.31 (2H, m), 7.26 (1H, d, J=7.2 Hz,), 7.20 (2H, d, J=6.8 Hz), 6.40 (1H, m), 6.36 (1H, m), 5.18 (2H, s), 4.10 (2H, s);

¹³C NMR (DMSO-d₆): δ 167.0, 151.2, 148.0, 143.1, 134.5, 128.9, 128.5, 127.0, 110.7, 109.2, 40.0, 30.9;

MS (ESI) m/z 272 (M+1).

### EXAMPLE 131

### 5-Benzyl-4-(3,5-dimethyl-isoxazol-4-yl)-2,4-dihydro-[1,2,4]triazole-3-thione

Starting with phenylacetic acid hydrazide (100 mg, 666 μmol) and 3,5-dimethyl4-isoxazolyl isothiocyanate (154 mg, 999 ,mol, obtained from Maybridge) afforded 113 mg (59%) of the title compound.

¹H NMR (DMSO-d₆) δ 13.97 (1H, s), 7.26 (3H, m), 7.02 (2H, m), 3.95 (1H, d, J=15.9 Hz), 3.84 (1H, d, J=15.9 Hz), 2.04 (3H, s), 1.65 (3H, s);

¹³C NMR (DMSO-d6) δ 168.6, 167.2, 157.6, 151.8, 134.2, 128.6, 127.2, 110.9, 31.1, 10.1, 8.6;

MS (ESI) m/z 287 (M+1).

### EXAMPLE 132

### 5-Benzyl4-(5-methyl-3-phenyl-isoxazol-4-yl)-2,4-dihydro-[1,2,4]triazole-3-thione

Starting with phenylacetic acid hydrazide (100 mg, 666 μmol) and 5-methyl-3-phenyl-4-isoxazolyl isothiocyanate (154 mg, 999 μmol, obtained from Maybridge) afforded 121 mg (52%) of the title compound.

¹H NMR (DMSO-d₆) δ 14.08 (1H, s), 7.52-7.42 (3H, m), 7.32 (2H, m), 7.17 (3H, m), 6,87 (2H, m), 3.82 (1H, d, J=15.9 Hz), 3.48 (1H, d, J=15.9 Hz), 1.95 (3H, s);

¹³C NMR (DMSO-d₆) δ 169.4, 168.9, 158.7, 151.4, 133.6, 130.6, 129.2, 128.5, 128.3, 127.2, 126.8, 126.4, 109.4, 31.2, 10.2;

MS (ESI) m/z 349 (M+1).

### EXAMPLE 133

### 4-(2,1,3-Benzothiadiazol4yl)-5-benzyl-2,4-dihydro-[1,2,4]triazole-3-thione

Starting with phenylacetic acid hydrazide (100 mg, 666 μmol) and 2,1,3-benzothiadiazol-4-yl isothiocyanate (193 mg, 999 μmol) afforded 92 mg (42%) of the title compound.

hu 1H NMR (DMSO-d₆) δ 13.95 (1H, s), 8.23 (1H, dd, J=8.6, 1.0 Hz), 7.82 (1H, dd, J=8.6, 7.1 Hz), 7.73 (1H, dd, J=7.1, 1.0 Hz), 6.98 (3H, m), 6.70 (2H, m), 3.87 (1H, d, J=16.2 Hz), 3.82 (1H, d, J=16.2 Hz);

¹³C NMR (DMSO-d₆) δ 168.6, 154.7, 151.5, 150.4, 133.9, 130.6, 129.5, 128.4, 127.9, 126.6, 125.3, 123.2, 31.4;

MS (ESI) m/z 326 (M+1).

### EXAMPLE 134

### 5-Benzyl4-pyridin-2-yl-2,4-dihydro-[1,2,4]triazole-3-thione

(Santus, M. Acta Poloniae Pharmaceutica 1980, 37, 293-300).

Starting with phenylacetic acid hydrazide (100 mg, 666 μmol) and 2-pyridyl isothiocyanate (136 mg, 999 μmol) afforded 125 mg (70%) of the title compound.

¹H NMR (DMSO-d₆) δ 13.88 (1H, s), 8.62 (1H, m), 7.94 (1H, dt, J=7.8, 1.8 Hz), 7.53 (1H, m), 7.43 (1H, d, J=8.1 Hz), 7.16 (3H, m), 6.91 (2H, m), 4.02 (2H, s);

¹³C NMR (DMSO-d₆) δ 167.4, 151.1, 149.2, 146.7, 138.6, 134.4, 128.5, 128.3, 126.8, 124.7, 123.5, 31.4;

MS (ESI) m/z 269 (M+1).

### EXAMPLE 135

### 5-Benzyl-4-(2-cyanophenyl)-2,4-dihydro-[1,2,4]triazole-3-thione

A suspension of 2-cyanophenyl isothiocyanate (300 mg, 1.87 mmol) and phenylacetic acid hydrazide (187 mg, 1.25 mmol) in isopropanol (5 ml) was run in a Smith Synthesizer microwave at 150° C. for 10 min. The reaction mixture was poured onto ice and the precipitated product was collected by filtration and washed with water. The product was dissolved in 2% NaOH (aq) and again precipitated by neutralization with 1M HCl. Recrystallization from ethyl acetate, followed by washing with warm methanol, afforded 83 mg (23%) of the title compound.

¹H NMR (DMSO-d₆): δ 13.93 (1H, br s), 8.17 (1H, m), 7.77 (1H, m), 7.64 (1H, d, J=8.20 Hz), 7.49 (1H, t, J=7.52 Hz), 7.35 (4H, m), 7.24 (1H, t, J=7.13 Hz), 4.25 (2H, s);

¹³C NMR (DMSO-d₆): δ 166.5, 166.1, 149.0, 137.3, 136.3, 133.0, 128.9, 128.4, 126.5, 125.4, 124.0, 116.3, 112.0, 34.2;

MS (ESI) m/z 293 (M+1).

### EXAMPLE 136

### 5-Benzyl-4-(2-thienyl)-2,4-dihydro-[1,2,4]triazole-3-thione

### (a) 5-Benzyl-4(2-thienyl)-2,4-dihydro-[1,2,4]triazol-3-one

A solution of 2-thienyl isocyanate (875 mg, 7.0 mmol) and phenylacetic acid hydrazide (700 mg, 4.66 mmol) in isopropanol (7 ml) and DMF (1 ml) was stirred under reflux overnight. The reaction mixture was cooled to ambient temperature and poured onto ice. The precipitated intermediate was filtered off and washed with water, then refluxed in 2% aqueous NaOH (5 ml) for 1 h. The reaction mixture was cooled to ambient temperature and neutralized with 1M HCl. The precipitate was collected by filtration and washed with water to afford 388 mg (32%) of the title compound.

¹H NMR (DMSO-d₆): δ 11.85 (1H, s), 7.53 (1H, dd, J=5.05, 1.77 Hz), 7.23 (3H, m), 7.03 (3H, m), 7.01 (1H, br s), 3.86 (2H, s);

¹³C NMR (DMSO-d₆): δ 154.1, 146.2, 134.9, 132.6, 128.4, 128.3, 126.7, 126.3, 126.0, 125.8, 31.8;

MS (ESI) m/z 258 (M+1).

### (b) 5-Benzyl-4-(2-thienyl)-2,4-dihydro-[1,2,4]triazole-3-thione

A suspension of 5-benzyl-4-(2-thienyl)-2,4-dihydro-[1,2,4]triazol-3-one (119 mg, 0.46 mmol) and Lawesson's reagent (281 mg, 0.69 mmol) in anhydrous toluene (5 ml) was run in a Smith Synthesizer microwave oven at 120° C. for 30 min. The solvent was evaporated and the residue dissolved in 10% aqueous NaOH. The resulting mixture was stirred for 1 h at ambient temperature and then filtered. The basic filtrate was treated with 1M HCl. The precipitated product was collected by filtration and purified by preparative HPLC to give 27 mg (23%) of the title compound.

¹H NMR (DMSO-d₆): δ 13.87 (1H, s), 7.65 (1H, dd, J=5.47, 1.56 Hz), 7.23 (3H, m), 7.12 (1H, m), 7.08 (1H, m), 6.98 (2H, dd, J=7.62, 1.76 Hz), 3.93 (2H, s);

¹³C NMR (DMSO-d₆): δ 169.0, 151.9, 134.4, 132.8, 128.5, 128.4, 127.9, 127.7, 126.9, 125.8, 31.3;

MS (ESI) m/z 258 (M+1).

### EXAMPLE 137

### 5-Benzyl-4-[2-(2-thienyl)ethyl]-2,4-dihydro-[1,2,4]triazole-3-thione

### (a) 5-Benzyl-4-[2-(2-thienyl)ethyl ]-2,4-dihydro-[1,2,4]triazol-3-one

A mixture of 2-thienyl isocyanate (311 mg, 2.0 mmol) and phenylacetic acid hydrazide (254 mg, 4.66 mmol) in isopropanol (3 ml) was run in a Smith Synthesizer microwave oven at 150° C. for 10 min. The reaction mixture was poured onto ice and the precipitated product collected by filtration. The precipitate was then dissolved in 2% aqueous NaOH and run in the Smith Synthesizer microwave oven at 120° C. for 10 min. The reaction mixture was neutralized with 1M HCl and the product was collected by filtration and washed with water. It was then dissolved in ethyl acetate, dried (MgSO₄) and concentrated in vacuo to afford 329 mg (68%) of the title compound.

¹H NMR (DMSO-d₆): δ 11.53 (1H, s), 7.37 (1H, m), 7.33 (2H, m), 7.27 (1H, m), 7.19 (2H, m), 6.96 (1H, m), 6.76 (1H, d, J=3.03 Hz, 1H), 3.68 (2H, s), 3.61 (2H, m), 2H, t, J=7.33 Hz);

¹³C NMR (DMSO-d₆): δ 154.9, 146.2, 139.6, 135.3, 128.7, 128.6, 127.2, 126.9, 125.9, 124.7, 42.1, 31.3, 27.9;

MS (ESI) m/z 286 (M+1).

### (b) 5-Benzyl-4-[2-(2-thienyl)ethyl]-2,4-dihydro-[1,2,4]triazole-3-thione

A suspension of 5-benzyl4-[2-(2-thienyl)ethyl]-2,4-dihydro-[1,2,4]triazol-3-one (300 mg, 1.05 mmol) and Lawesson's reagent (1.275 g, 3.15 mmol) in anhydrous toluene (5 ml) was run in a Smith Synthesizer microwave oven at 150 ° C. for 30 min. The solvent was evaporated and the residue dissolved in 10% aqueous NaOH. The resulting mixture was stirred for 1 h at ambient temperature and then filtered. The filtrate was treated with 1M HCl and extracted with ethyl acetate. The product that had not dissolved in 10% aqueous NaOH was partitioned between 1M HCl and ethyl acetate. The combined organic phases were dried (MgSO4) and concentrated in vacuo. Purification was achieved by flash chromatography using a heptane-ethyl acetate gradient followed by preparative HPLC, and afforded 65 mg (21%) of the title compound.

¹H NMR (DMSO-d₆): δ 13.60 (1H, br s), 7.40 (1H, dd, J=5.08, 1.17 Hz), 7.35 (2H, m), 7.27 (1H, m), 7.18 (2H, d, J=7.03 Hz), 6.98 (1H, dd, J=5.18, 3.42 Hz), 6.78 (1H, d, j=2.73 Hz), 4.00 (2H, m), 3.80 (2H, s), 2.91 (2H, m);

¹³C NMR (DMSO-d₆): δ 166.5, 151.3, 139.3, 134.8, 128.8, 128.8, 127.3, 127.1, 126.1, 124.9,44.9, 30.5, 26.7;

MS (ESI) m/z 302 (M+1).

### EXAMPLE 138

### 5-(2-Chlorobenzyl)-4-(3-diethylaminopropyl)-2,4-dihydro-[1,2,4]triazole-3-thione

Diethyl-(3-isothiocyanato-propyl)-amine (125 mg, 0.73 mmol) was added dropwise to a solution of (2-chlorophenyl)-acetic acid hydrazide (122 mg, 0.66 mmol) in isopropanol (5 ml). The resulting reaction mixture was heated at 60° C. for 4 h and then at 75° C. for 2 h. The reaction mixture was concentrated in vacuo and the product purified by flash chromatography (chloroform-methanol gradient), to yield 101 mg (45%) of the title compound.

¹H NMR (CDCl₃) δ 8.84 (1H, br s), 7.39 (1H, m), 7.22 (2H, m), 7.12 (1H, m), 4.19 (2H, s), 3.91 (2H, m), 2.51 (6H, quintet, J=7.1 Hz), 1.86 (2H, m), 0.99 (6H, t, J=7.1 Hz);

¹³C NMR (CDCl₃) δ 167.7, 150.5, 133.8, 132.0, 130.3, 129.8, 129.1, 127.3, 49.4, 46.2, 42.7, 29.3, 25.2, 11.1;

MS (ESI) m/z 339 (M+1).

### Screens

Methods for the determination of MPO inhibitory activity are disclosed in co-pending patent application WO 02/090575. The pharmacological activity of compounds according to the invention was tested in the following screen in which the compounds were either tested alone or in the presence of added tyrosine:

Assay buffer: 20 mM sodium/potassium phosphate buffer pH 6.5 containing 10 mM taurine and 100 mM NaCl.

Developing reagent: 2 mM 3,3 ′,5,5′-tetramethylbenzidine (TMB), 200 μM KI, 200 mM acetate buffer pH 5.4 with 20% DNF.

To 10 μl of diluted compounds in assay buffer, 40 μl of human MPO (final concentration 2.5 nM), with or without 20 μM tyrosine (final concentration, if present, 8 uM), was added and the mixture was incubated for 10 minutes at ambient temperature. Then 50 μl of H₂O₂ (final concentration 100 μM), or assay buffer alone as a control, were added. After incubation for 10 minutes at ambient temperature, the reaction was stopped by adding 10 μl 0.2 mg/ml of catalase (final concentration 18 μg/ml). The reaction mixture was left for an additional 5 minutes before 100 μl of TMB developing reagent was added. The amount of oxidised 3,3′,5,5′-tetramethylbenzidine formed was then measured after about 5 minutes using absorbance spectroscopy at about 650 nM. IC₅₀ values were then determined using standard procedures.

When tested in at least one version of the above screen, the compounds of Examples 1 to 138 gave IC₅₀ values of less than 60 μM, indicating that they are expected to show useful therapeutic activity. Representative results are shown in the following Table.

| | Inhibition of MPO |
|---|---|
| | (in the presence of tyrosine) |
| Compound | IC₅₀ μM |
| Example 20 | 11.3 |
| Example 29 | 3.9 |
| Example 57 | 23.6 |
| Example 74 | 7.2 |

1-13. (canceled)

14. A method of treating neuroinflammatory disorders comprising administering to a subject in need thereof a compound of formula (I): 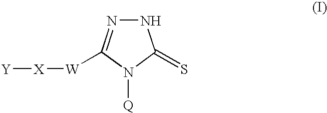
wherein:
Q represents phenyl, naphthyl or a monocyclic or bicyclic heteroaromatic ring system containing one to three heteroatoms independently selected from O, N and S; said phenyl, naphthyl or heteroaromatic ring being optionally substituted by one to three substituents independently selected from halogen, CN, C1 to 6 alkyl, C1 to 6 alkoxy, C1 to 6 alkylthio, CO₂R⁶, COR⁷, CH₂OH, Ph, NO₂, NR⁸R⁹ and SO₂NR¹⁰R¹¹; said alkyl or alkoxy group being optionally further substituted by one or more fluoro atoms;
or Q represents C1 to 6 alkyl optionally substituted by one or more groups independently selected from C1 to 6 alkoxy, NR⁸R⁹, phenyl, a 5- or 6-membered heteroaromatic ring containing one or two heteroatoms independently selected from O, S and N, or a 5- or 6-membered saturated heterocyclic ring containing one or two heteroatoms independently selected from O, N and S;
or Q represents C3 to 8 cycloalkyl;
W represents a bond or CHR¹ wherein R¹ represents H, CH₃, F, OH, CH₂OH or Ph;
X represents a bond, O, CH₂ or NR³ wherein R³ represents H or C1 to 6 alkyl;
Y represents phenyl, naphthyl or a monocyclic or bicyclic heteroaromatic ring system containing one to three heteroatoms independently selected from O, N and S; said phenyl, naphthyl or heteroaromatic ring system being optionally substituted by one to three substituents independently selected from halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
or Y represents C1 to 6 alkyl or C3 to 6 cycloalkyl; said cycloalkyl group optionally including an O atom and optionally being benzo fused; and said alkyl or cycloalkyl group being optionally substituted by one or more substituents independently selected from halogen, oxo (═O), C1 to 6 alkyl or C1 to 6 alkoxy;
each R⁴, R⁵, R⁶, R⁷, R¹² and R¹³ independently represents H or C1 to 6 alkyl;
each R⁸, R⁹, R¹⁰ and R¹¹ independently represents H or C1 to 6 alkyl; or the group NR⁸R⁹ or NR¹⁰R¹¹ together represents a saturated 5- or 6-membered azacyclic ring optionally including one further heteroatom selected from O, S and N, and optionally being substituted by one or more C1 to 6 alkyl groups;
or a pharmaceutically acceptable salt thereof, in the manufacture of a medicament, for the treatment or prophylaxis of diseases or conditions in which inhibition of the enzyme MPO is beneficial.

15. A method according to claim 14 wherein Q represents phenyl optionally substituted by halogen, C1 to 6 alkyl or C1 to 6 alkoxy.

16. A method according to claim 14 wherein Y represents optionally substituted phenyl.

17. A method according to claim 14 wherein W represents a bond or CH₂.

18. A method according to claim 14 wherein X represents a bond or O.

19. A pharmaceutical formulation comprising a therapeutically effective amount of a compound of formula (I), according to claim 14, or a pharmaceutically acceptable salt thereof, in a mixture with a pharmaceutically acceptable adjuvant, diluent or carrier, for use in the treatment or prophylaxis of neuroinflammatory disorders.

20. A method according to claim 14 where said compound is selected from:
5-(4-aminobenzyl)-4-[3,5-di(trifluoromethyl)phenyl]-2,4-dihydro-[1,2,4]triazole-3-thione;
5-(4-chlorobenzyl)-4-(4-methylphenyl)-2,4-dihydro-[1,2,4]triazole-3-thione;
5-isobutyl-4-phenyl-2,4-dihydro-[1,2,4]triazole-3-thione;
5-(4-hydroxybenzyl)-4-[3-(methylthio)phenyl]-2,4-dihydro-[1,2,4]triazole-3-thione;
5-(2,5-dimethoxybenzyl)-4-[3-(methylthio)phenyl]-2,4-dihydro-[1,2,4]triazole-3-thione;
5-(4-hydroxybenzyl)-4-(4-iodophenyl)-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(4-hydroxybenzyl)-4-[4-(piperidinosulfonyl)phenyl]-2,4-dihydro-[1,2,4]triazole-3-thione;
5-(2,5-dimethoxybenzyl)-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
5-(4-hydroxybenzyl)-4-(2,4,6-trichlorophenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
4-(4-carboxyphenyl)-5-(diphenylmethyl)-2,4-dihydro-[1,2,4]triazole-3-thione;
5-(2-bromobenzyl)-4-(4-sulfamoylphenyl)-2,4-dihydro-[1,2,4]triazole-3-thione;
5-(4-hydroxybenzyl)-4-(naphthalen-1-yl)-2,4-dihydro-[1,2,4]triazole-3-thione;
5-(4-hydroxybenzyl)-4-(2,6-dibromo-4-methylphenyl)-2,4-dihydro-[1,2,4]triazole-3-thione;
5-(4-hydroxybenzyl)-4-(3,4,5-trimethoxyphenyl)-2,4-dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(4-methoxyphenoxymethyl)-4-(2-tetrahydrofuran-2-yl-methyl)-2,4-dihydro-[1,2,4]triazole-3-thione;
5-(4-methoxyphenoxymethyl)-4-(3-methoxypropyl)-2,4-dihydro-[1,2,4]triazole-3-thione;
5-(4-methoxyphenoxymethyl)-4-(2-phenylethyl)-2,4-dihydro-[1,2,4]triazole-3-thione;
5-(4-methoxyphenoxymethyl)-4-(3-morpholin-4-yl-propyl)-2,4-dihydro-[1,2,4]triazole-3-thione;
4-butyl-5-[(4-methoxyphenylamino)-methyl]-2,4-dihydro-[1,2,4]triazole-3-thione;
5-[(4-methoxyphenylamino)-methyl]-4-(3-methoxypropyl)-2,4-dihydro-[1,2,4]triazole-3-thione;
4-hexyl-5-[(4-methoxyphenylamino)methyl]-2,4-dihydro-[1,2,4]triazole-3-thione;
5-(2-chlorobenzyl)-4-(2-ethoxyphenyl)-2,4-dihydro-[1,2,4]triazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-pyridin-3-yl-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(4-methoxyphenoxymethyl)-4-cyclopropyl-2,4-dihydro-[1,2,4]triazole-3-thione;
4-(2,2-dimethoxyethyl)-5-[(4-methoxyphenylamino)methyl]-2,4-dihydro-[1,2,4]triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4-dihydro-[1,2,4]triazole-3-thione;
5-(2-chlorobenzyl)-4-(3-methylphenyl)-2,4-dihydro-[1,2,4]triazole-3-thione;
5-[(2-chlorophenyl)hydroxymethyl]-4-isobutyl-2,4-dihydro-[1,2,4]triazole-3-thione;
5-[(2-chloro-phenyl)hydroxymethyl]-4-cyclooctyl-2,4-dihydro-[1,2,4]triazole-3-thione;
5-[(2-chlorophenyl)hydroxymethyl]-4-(2,2-dimethoxyethyl)-2,4-dihydro-[1,2,4]triazole-3-thione;
5-[(2-chlorophenyl)hydroxymethyl]-4-(2-methylbutyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro-[1,2,4]triazole-3-thione;
4-phenyl-5-(pyrrol-2-yl)-2,4-dihydro-[1,2,4]triazole-3-thione;
5-(2-chlorobenzyl)-4-(3-hydroxymethylphenyl)-2,4-dihydro-[1,2,4]triazole-3-thione;
5-(4-chlorobenzyl)-4-phenyl-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
4-phenyl-5-pyridin-3-yl-methyl-2,4-dihydro-[1,2,4]triazole-3-thione;
5-(3-chlorobenzyl)-4-(2-methoxyphenyl)-2,4-dihydro-[1,2,4]triazole-3-thione;
5-(3-methoxybenzyl)-4-phenyl1-2,4-dihydro-[1,2,4]triazole-3-thione;
5-(2-bromo-5-methylbenzyl)-4-phenyl-2,4-dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(furan-2-ylmethyl)-4-phenyl-2,4-dihydro-[1,2,4]triazole-3-thione;
5-(3-methylbenzyl)-4-phenyl-2,4-dihydro-[1,2,4]triazole-3-thione;
5-(2-chlorobenzyl)-4-(4-methylphenyl)-2,4-dihydro-[1,2,4]triazole-3-thione;
5-(2-hydroxy-1-phenylethyl)-4-phenyl-2,4-dihydro-[1,2,4]triazole-3-thione;
5-(3,5-dimethylbenzyl)-4-phenyl-2,4-dihydro-[1,2,4]triazole-3-thione;
5-(2,3-dichlorobenzyl)-4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(3-trifluoromethylbenzyl)-4-phenyl-2,4-dihydro-[1,2,4]triazole-3-thione;
5-phenoxy-4-phenyl-2,4-dihydro-[1,2,4]triazole-3-thione;
5-(2-methylbenzyl)-4-phenyl-2,4-dihydro-[1,2,4]triazole-3-thione;
5-[(2-chlorophenyl)hydroxymethyl]-4-cyclohexyl-2,4-dihydro-[1,2,4]triazole-3-thione;
5-[(2-chlorophenyl)hydroxymethyl]-4-phenyl-2,4-dihydro-[1,2,4]triazole-3-thione;
5-[(2-chlorophenyl)hydroxymethyl]-4-(3-methoxypropyl)-2,4-dihydro-[1,2,4]triazole-3-thione;
5-(2-chlorobenzyl)-4-(2-piperidin-1-yl-ethyl)-2,4-dihydro-[1,2,4]triazole-3-thione;
4-butyl-5-(2-chlorobenzyl)-2,4-dihydro-[1,2,4]triazole-3-thione;
5-(2-chlorobenzyl)-4-(3-morpholin-4-yl-propyl)-2,4-dihydro-[1,2,4]triazole-3-thione;
5-(2-chlorobenzyl)-4-(tetrahydrofuran-2-yl-methyl)-2,4-dihydro-[1,2,4]triazole-3-thione;
5-(2-chlorobenzyl)-4-(4-chlorophenyl)-2,4-dihydro-[1,2,4]triazole-3-thione;
5-(1H-indol-3-ylmethyl)-4-(2-methoxyphenyl)-2,4-dihydro-[1,2,4]triazole-3-thione;
5-(1H-indol-3-ylmethyl)-4-(2-methylphenyl)-2,4-dihydro-[1,2,4]triazole-3-thione;
5-cyclopentylmethyl-4-phenyl-2,4-dihydro-[1,2,4]triazole-3-thione;
5-(2-chlorobenzyl-4-(2-chlorophenyl)-2,4-dihydro-[1,2,4]triazole-3-thione;
5-[(2-chlorophenyl)hydroxymethyl]-4-(4-nitrophenyl)-2,4-dihydro-[1,2,4]triazole-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(3-butoxybenzyl)-4-phenyl-2,4-dihydro-[1,2,4]triazole-3-thione;
5-(2-chloro-5-methoxybenzyl)-4-phenyl-2,4-dihydro-[1,2,4]triazole-3-thione;
5-(3-chlorobenzyl)-4-o-tolyl-2,4-dihydro-[1,2,4]triazole-3-thione;
5-(6-chloro-2-fluoro-3-methylbenzyl)-4-phenyl-2,4-dihydro-[1,2,4]triazole-3-thione;
5-(biphenyl-2-ylmethyl)-4-phenyl-2,4-dihydro-[1,2,4]triazole-3-thione;
5-(3-oxo-indan-1-yl)-4-phenyl-2,4-dihydro-[1,2,4]triazole-3-thione;
5-(4-acetylphenoxy)methyl-4-phenyl-2,4-dihydro-[1,2,4]triazole-3-thione;
5-(3-methoxyphenoxy)methyl-4-phenyl-2,4-dihydro-[1,2,4]triazole-3-thione;
5-(4-butoxyphenoxy)methyl-4-phenyl-2,4-dihydro-[1,2,4]triazole-3-thione;
5-(3-chlorophenoxy)methyl-4-phenyl-2,4-dihydro-[1,2,4]triazole-3-thione;
5-(2-methylcarbamoylphenoxy)methyl-4-phenyl-2,4-dihydro-[1,2,4]triazole-3-thione;
5-(3-butoxy-phenoxymethyl)-4-phenyl-2,4-dihydro-[1,2,4]triazole-3-thione;
5-isochroman-1-yl-4-phenyl-2,4-dihydro-[1,2,4]triazole-3-thione;
5-{3-[(methylamino)carbonyl]benzyl}-4-phenyl-2,4-dihydro-[1,2,4]triazole-3-thione;
4-phenyl-5-(pyridin-2-ylmethyl)-2,4-dihydro-[1,2,4]triazole-3-thione;
5-(2,3-dimethoxybenzyl)-4-phenyl-2,4-dihydro-[1,2,4]triazole-3-thione;
4-phenyl-5-(2,3,4-trimethoxybenzyl)-2,4-dihydro-[1,2,4]-triazole-3-thione;
5-[(2,5-dimethyl-1,3-thiazol-4-yl)methyl]-4-phenyl-2,4-dihydro-[1,2,4]triazole-3-thione;
5-[(2-butoxyphenoxy)methyl]-4-phenyl-2,4-dihydro-[1,2,4]triazole-3-thione;
4-phenyl-5-(tetrahydrofuran-2-ylmethyl)-2,4-dihydro-[1,2,4]triazole-3-thione;
4-[4-(2,6-dimethyl-morpholin-4-yl)-phenyl]-5-(diphenylmethyl)-2,4-dihydro-[1,2,4]triazole-3-thione;
5-benzyl-4-(2-furylmethyl)-2,4-dihydro-[1,2,4]triazole-3-thione;
5-benzyl-4-(3,5-dimethyl-isoxazol-4-yl)-2,4-dihydro-[1,2,4]triazole-3-thione;
5-benzyl-4-(5-methyl-3-phenyl-isoxazol-4-yl)-2,4-dihydro-[1,2,4]triazole-3-thione;
4-(2,1,3-benzothiadiazol-4-yl)-5-benzyl-2,4-dihydro-[1,2,4]triazole-3-thione;
5-benzyl-4-(2-cyanophenyl)-2,4-dihydro-[1,2,4]triazole-3-thione;
5-benzyl-4-(2-thienyl)-2,4-dihydro-[1,2,4]triazole-3-thione;
5-benzyl-4-[2-(2-thienyl)ethyl]-2,4-dihydro-[1,2,4]triazole-3-thione; or
5-(2-chlorobenzyl)-4-(3-diethylaminopropyl)-2,4-dihydro-[1,2,4]triazole-3-thione;
or a pharmaceutically acceptable salt thereof.

21. A method of treating multiple sclerosis in a subject comprising administering to such subject an effective amount of a compound of formula I 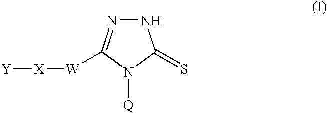
wherein:
Q represents phenyl, naphthyl or a monocyclic or bicyclic heteroaromatic ring system containing one to three heteroatoms independently selected from O, N and S; said phenyl, naphthyl or heteroaromatic ring being optionally substituted by one to three substituents independently selected from halogen, CN, C1 to 6 alkyl, C1 to 6 alkoxy, C1 to 6 alkylthio, CO₂R⁶, COR⁷, CH₂OH, Ph, NO₂, NR⁸R⁹ and SO₂NR¹⁰R¹¹; said alkyl or alkoxy group being optionally further substituted by one or more fluoro atoms;
or Q represents C1 to 6 alkyl optionally substituted by one or more groups independently selected from C1 to 6 alkoxy, NR⁸R⁹, phenyl, a 5- or 6-membered heteroaromatic ring containing one or two heteroatoms independently selected from O, S and N, or a 5- or 6-membered saturated heterocyclic ring containing one or two heteroatoms independently selected from O, N and S;
or Q represents C3 to 8 cycloalkyl;
W represents a bond or CHR¹ wherein R¹ represents H, CH₃, F, OH, CH₂OH or Ph;
X represents a bond, O, CH₂ or NR³ wherein R³ represents H or C1 to 6 alkyl;
Y represents phenyl, naphthyl or a monocyclic or bicyclic heteroaromatic ring system containing one to three heteroatoms independently selected from O, N and S; said phenyl, naphthyl or heteroaromatic ring system being optionally substituted by one to three substituents independently selected from halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
or Y represents C1 to 6 alkyl or C3 to 6 cycloalkyl; said cycloalkyl group optionally including an O atom and optionally being benzo fused; and said alkyl or cycloalkyl group being optionally substituted by one or more substituents independently selected from halogen, oxo (═O), C1 to 6 alkyl or C1 to 6 alkoxy;
each R⁴, R⁵, R⁶, R⁷, R¹² and R¹³ independently represents H or C1 to 6 alkyl;
each R⁸, R⁹, R¹⁰ and R¹¹ independently represents H or C1 to 6 alkyl; or the group NR⁸R⁹ or NR¹⁰R¹¹ together represents a saturated 5- or 6-membered azacyclic ring optionally including one further heteroatom selected from O, S and N, and optionally being substituted by one or more C1 to 6 alkyl groups;
or a pharmaceutically acceptable salt thereof.

22. A method of treating multiple sclerosis in a subject comprising administering to such subject an effective amount of a compound selected from:
5-(4-aminobenzyl)-4-[3,5-di(trifluoromethyl)phenyl]-2,4-dihydro-[1,2,4]triazole-3-thiune;
5-(4-chlorobenzyl)-4-(4-methylphenyl)-2,4-dihydro-[1,2,4]triazole-3-thione;
5-isobutyl-4-phenyl-2,4-dihydro-[1,2,4]triazole-3-thione;
5-(4-hydroxybenzyl)-4-[3-(methylthio)phenyl]-2,4-dihydro-[1,2,4]triazole-3-thione;
5-(2,5-dimethoxybenzyl)-4-[3-(methylthio)phenyl]-2,4-dihydro-[1,2,4]triazole-3-thione;
5-(4-hydroxybenzyl)-4-(4-iodophenyl)-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(4-hydroxybenzyl)-4-[4-(piperidinosulfonyl)phenyl]-2,4-dihydro-[1,2,4]triazole-3-thione;
5-(2,5-dimethoxybenzyl)-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
5-(4-hydroxybenzyl)-4-(2,4,6-trichlorophenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
4-(4-carboxyphenyl)-5-(diphenylmethyl)-2,4-dihydro-[1,2,4]triazole-3-thione;
5-(2-bromobenzyl)-4-(4-sulfamoylphenyl)-2,4-dihydro-[1,2,4]triazole-3-thione;
5-(4-hydroxybenzyl)-4-(naphthalen-1-yl)-2,4-dihydro-[1,2,4]triazole-3-thione;
5-(4-hydroxybenzyl)-4-(2,6-dibromo-4-methylphenyl)-2,4-dihydro-[1,2,4]triazole-3-thione;
5-(4-hydroxybenzyl)-4-(3,4,5-trimethoxyphenyl)-2,4-dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(4-methoxyphenoxymethyl)-4-(2-tetrahydrofuran-2-yl-methyl)-2,4-dihydro-[1,2,4]triazole-3-thione;
5-(4-methoxyphenoxymethyl)-4-(3-methoxypropyl)-2,4-dihydro-[1,2,4]triazole-3-thione;
5-(4-methoxyphenoxymethyl)-4-(2-phenylethyl)-2,4-dihydro-[1,2,4]triazole-3-thione;
5-(4-methoxyphenoxymethyl)-4-(3-morpholin-4-yl-propyl)-2,4-dihydro-[1,2,4]triazole-3-thione;
4-butyl-5-[(4-methoxyphenylamino)-methyl]-2,4-dihydro-[1,2,4]triazole-3-thione;
5-[(4-methoxyphenylamino)-methyl]-4-(3-methoxypropyl)-2,4-dihydro-[1,2,4]triazole-3-thione;
4-hexyl-5-[(4-methoxyphenylamino)methyl]-2,4-dihydro-[1,2,4]triazole-3-thione;
5-(2-chlorobenzyl)-4-(2-ethoxyphenyl)-2,4-dihydro-[1,2,4]triazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-pyridin-3-yl-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(4-methoxyphenoxymethyl)-4-cyclopropyl-2,4-dihydro-[1,2,4]triazole-3-thione;
4-(2,2-dimethoxyethyl)-5-[(4-methoxyphenylamino)methyl]-2,4-dihydro-[1,2,4]triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4-dihydro-[1,2,4]triazole-3-thione;
5-(2-chlorobenzyl)-4-(3-methylphenyl)-2,4-dihydro-[1,2,4]triazole-3-thione;
5-[(2-chlorophenyl)hydroxymethyl]-4-isobutyl-2,4-dihydro-[1,2,4]triazole-3-thione;
5-[(2-chloro-phenyl)hydroxymethyl]-4-cyclooctyl-2,4-dihydro-[1,2,4]triazole-3-thione;
5-[(2-chlorophenyl)hydroxymethyl]-4-(2,2-dimethoxyethyl)-2,4-dihydro-[1,2,4]triazole-3-thione;
5-[(2-chlorophenyl)hydroxymethyl]-4-(2-methylbutyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro-[1,2,4]triazole-3-thione;
4-phenyl-5-(pyrrol-2-yl)-2,4-dihydro-[1,2,4]triazole-3-thione;.
5-(2-chlorobenzyl)-4-(3-hydroxymethylphenyl)-2,4-dihydro-[1,2,4]triazole-3-thione;
5-(4-chlorobenzyl)-4-phenyl-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
4-phenyl-5-pyridin-3-yl-methyl-2,4-dihydro-[1,2,4]triazole-3-thione;
5-(3-chlorobenzyl)-4-(2-methoxyphenyl)-2,4-dihydro-[1,2,4]triazole-3-thione;
5-(3-methoxybenzyl)-4-phenyl-2,4-dihydro-[1,2,4]triazole-3-thione;
5-(2-bromo-5-methylbenzyl)-4-phenyl-2,4-dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(furan-2-ylmethyl)-4-phenyl-2,4-dihydro-[1,2,4]triazole-3-thione;
5-(3-methylbenzyl)-4-phenyl-2,4-dihydro-[1,2,4]triazole-3-thione;
5-(2-chlorobenzyl)-4-(4-methylphenyl)-2,4-dihydro-[1,2,4]triazole-3-thione;
5-(2-hydroxy-1-phenylethyl)-4-phenyl-2,4-dihydro-[1,2,4]triazole-3-thione;
5-(3,5-dimethylbenzyl)-4-phenyl-2,4-dihydro-[1,2,4]triazole-3-thione;
5-(2,3-dichlorobenzyl)-4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(3-trifluoromethylbenzyl)-4-phenyl-2,4-dihydro-[1,2,4]triazole-3-thione;
5-phenoxy-4-phenyl-2,4-dihydro-[1,2,4]triazole-3-thione;
5-(2-methylbenzyl)-4-phenyl-2,4-dihydro-[1,2,4]triazole-3-thione;
5-[(2-chlorophenyl)hydroxymethyl]-4-cyclohexyl-2,4-dihydro-[1,2,4]triazole-3-thione;
5-[(2-chlorophenyl)hydroxymethyl]-4-phenyl-2,4-dihydro-[1,2,4]triazole-3-thione;
5-[(2-chlorophenyl)hydroxymethyl]-4-(3-methoxypropyl)-2,4-dihydro-[1,2,4]triazole-3-thione;
5-(2-chlorobenzyl)-4-(2-piperidin-1-yl-ethyl)-2,4-dihydro-[1,2,4]triazole-3-thione;
4-butyl-5-(2-chlorobenzyl)-2,4-dihydro-[1,2,4]triazole-3-thione;
5-(2-chlorobenzyl)-4-(3-morpholin-4-yl-propyl)-2,4-dihydro-[1,2,4]triazole-3-thione;
5-(2-chlorobenzyl)-4-(tetrahydrofuran-2-yl-methyl)-2,4-dihydro-[1,2,4]triazole-3-thione;
5-(2-chlorobenzyl)-4-(4-chlorophenyl)-2,4-dihydro-[1,2,4]triazole-3-thione;
5-(1H-indol-3-ylmethyl)-4-(2-methoxyphenyl)-2,4-dihydro-[1,2,4]triazole-3-thione;
5-(1H-indol-3-ylmethyl)-4-(2-methylphenyl)-2,4-dihydro-[1,2,4]triazole-3-thione;
5-cyclopentylmethyl-4-phenyl-2,4-dihydro-[1,2,4]triazole-3-thione;
5-(2-chlorobenzyl)-4-(2-chlorophenyl)-2,4-dihydro-[1,2,4]triazole-3-thione;
5-[(2-chlorophenyl)hydroxymethyl]-4-(4-nitrophenyl)-2,4-dihydro-[1,2,4]triazole-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(3-butoxybenzyl)-4-phenyl-2,4-dihydro-[1,2,4]triazole-3-thione;
5-(2-chloro-5-methoxybenzyl)-4-phenyl-2,4-dihydro-[1,2,4]triazole-3-thione;
5-(3-chlorobenzyl)-4-o-tolyl-2,4-dihydro-[1,2,4]triazole-3-thione;
5-(6-chloro-2-fluoro-3-methylbenzyl)-4-phenyl-2,4-dihydro-[1,2,4]triazole-3-thione;
5-(biphenyl-2-ylmethyl)-4-phenyl-2,4-dihydro-[1,2,4]triazole-3-thione;
5-(3-oxo-indan-1-yl)-4-phenyl-2,4-dihydro-[1,2,4]triazole-3-thione;
5-(4-acetylphenoxy)methyl-4-phenyl-2,4-dihydro-[1,2,4]triazole-3-thione;
5-(3-methoxyphenoxy)methyl-4-phenyl-2,4-dihydro-[1,2,4]triazole-3-thione;
5-(4-butoxyphenoxy)methyl-4-phenyl-2,4-dihydro-[1,2,4]triazole-3-thione;
5-(3-chlorophenoxy)methyl-4-phenyl-2,4-dihydro-[1,2,4]triazole-3-thione;
5-(2-methylcarbamoylphenoxy)methyl-4-phenyl-2,4-dihydro-[1,2,4]triazole-3-thione;
5-(3-butoxy-phenoxymethyl)-4-phenyl-2,4-dihydro-[1,2,4]triazole-3-thione;
5-isochroman-1-yl-4-phenyl-2,4-dihydro-[1,2,4]triazole-3-thione;
5-{3-[(methylamino)carbonyl]benzyl}-4-phenyl-2,4-dihydro-[1,2,4]triazole-3-thione;
4-phenyl-5-(pyridin-2-ylmethyl)-2,4-dihydro-[1,2,4]triazole-3-thione;
5-(2,3-dimethoxybenzyl)-4-phenyl-2,4-dihydro-[1,2,4]triazole-3-thione;
4-phenyl-5-(2,3,4-trimethoxybenzyl)-2,4-dihydro-[1,2,4]-triazole-3-thione;
5-[(2,5-dimethyl-1,3-thiazol-4-yl)methyl]-4-phenyl-2,4-dihydro-[1,2,4]triazole-3-thione;
5-[(2-butoxyphenoxy)methyl]-4-phenyl-2,4-dihydro-[1,2,4]triazole-3-thione;
4-phenyl-5-(tetrahydrofuran-2-ylmethyl)-2,4-dihydro-[1,2,4]triazole-3-thione;
4-[4-(2,6-dimethyl-morpholin-4-yl)-phenyl]-5-(diphenylmethyl)-2,4-dihydro-[1,2,4]triazole-3-thione;
5-benzyl-4-(2-furylmethyl)-2,4-dihydro-[1,2,4]triazole-3-thione;
5-benzyl-4-(3,5-dimethyl-isoxazol-4-yl)-2,4-dihydro-[1,2,4]triazole-3-thione;
5-benzyl-4-(5-methyl-3-phenyl-isoxazol-4-yl)-2,4-dihydro-[1,2,4]triazole-3-thione;
4-(2,1,3-benzothiadiazol-4-yl)-5-benzyl-2,4-dihydro-[1,2,4]triazole-3-thione;
5-benzyl-4-(2-cyanophenyl)-2,4-dihydro-[1,2,4]triazole-3-thione;
5-benzyl-4-(2-thienyl)-2,4-dihydro-[1,2,4]triazole-3-thione;
5-benzyl-4-[2-(2-thienyl)ethyl]-2,4-dihydro-[1,2,4]triazole-3-thione; or
5-(2-chlorobenzyl)-4-(3-diethylaminopropyl)-2,4-dihydro-[1,2,4]triazole-3-thione;
or a pharmaceutically acceptable salt thereof.

23. A compound of formula (Ia) 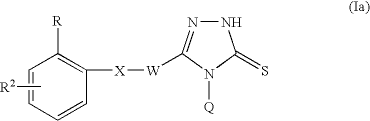
wherein:
Q represents phenyl optionally substituted by one to three substituents independently selected from halogen, CN, C1 to 6 alkyl, C1 to 6 alkoxy, C1 to 6 alkylthio, CO₂^{R}⁶, COR⁷, CH₂OH, Ph, NO₂, NR⁸R⁹ and SO₂NR¹⁰R¹¹; said alkyl or alkoxy group being optionally further substituted by one or more fluoro atoms;
W represents CH₂;
X represents a bond;
R represents halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
R² represents H or one or more substituents independently selected from halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
each R⁴, R⁵, R⁶, R⁷, R¹² and R¹³ independently represents H or C1 to 6 alkyl;
each R⁸, R⁹, R¹⁰ and R¹¹ independently represents H or C1 to 6 alkyl; or the group NR⁸R⁹ or NR¹⁰R¹¹ together represents a saturated 5- or 6-membered azacyclic ring optionally including one further heteroatom selected from O, S and N, and optionally being substituted by one or more C1 to 6 alkyl groups;
and pharmaceutically acceptable salts thereof; with the proviso that the following compounds are excluded:
5-[(2-chlorophenyl)methyl]-2,4-dihydro-4-phenyl-3H-1,2,4-triazole-3-thione;
5-[(2-chloro-6-fluorophenyl)methyl]-2,4-dihydro-4-phenyl-3H-1,2,4-triazole-3-thione.

24. A compound according to claim 23 which is:
5-(2,5-dimethoxybenzyl)-4-[3-(methylthio)phenyl]-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(2,5-dimethoxybenzyl)-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
5-(2-bromobenzyl)-4-(4-sulfamoylphenyl)-2,4-dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(2-chlorobenzyl)-4-(2-ethoxyphenyl)-2,4-dihydro-[1,2,4]triazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4-dihydro-[1,2,4]triazole-3-thione;
5-(2-chlorobenzyl)-4-(3-methylphenyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro-[1,2,4]triazole-3-thione;
5-(2-chlorobenzyl)-4-(3-hydroxymethylphenyl)-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
5-(2-bromo-5-methylbenzyl)-4-phenyl-2,4-dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(2-chlorobenzyl)-4-(4-methylphenyl)-2,4-dihydro-[1,2,4]triazole-3-thione;
5-(2,3-dichlorobenzyl)-4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(2-methylbenzyl)-4-phenyl-2,4-dihydro-[1,2,4]triazole-3-thione;
5-(2-chlorobenzyl)-4-(4-chlorophenyl)-2,4-dihydro-[1,2,4]triazole-3-thione;
5-(2-chlorobenzyl)-4-(2-chlorophenyl)-2,4-dihydro-[1,2,4]triazole-3-thione;
5-(2-chlorobenzyl)-4-phenyl-2,4-dihydro-[1,2,4]triazole-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(2-chloro-5-methoxybenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(2,3-dimethoxybenzyl)-4-phenyl-2,4-dihydro-[1,2,4]triazole-3-thione; or
4-phenyl-5-(2,3,4-trimethoxybenzyl)-2,4-dihydro-[1,2,4]-triazole-3-thione;
or a pharmaceutically acceptable salt thereof.

25. A pharmaceutical composition comprising a compound of formula (Ia) according to claim 20, or a pharmaceutically acceptable salt thereof, in a mixture with a pharmaceutically acceptable adjuvant, diluent or carrier.

26. A method of treating multiple sclerosis in a subject comprising administering to such subject an effective amount of a compound of formula (Ia): 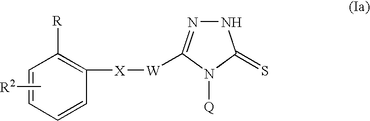
wherein:
Q represents phenyl optionally substituted by one to three substituents independently selected from halogen, CN, C1 to 6 alkyl, C1 to 6 alkoxy, C1 to 6 alkylthio, CO₂R⁶, COR⁷, CH₂OH, Ph, NO₂, NR⁸R⁹ and SO₂NR¹⁰R¹¹; said alkyl or alkoxy group being optionally further substituted by one or more fluoro atoms;
W represents CH₂;
X represents a bond;
R represents halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
R² represents H or one or more substituents independently selected from halogen, OH, C1 to 6 alkyl, C3 to 6 cycloalkyl, C1 to 6 alkoxy, C1 to 6 alkylthio, CO₂H, C2 to 6 alkanoyl, Ph, NO₂, C(O)NR¹²R¹³ or NR⁴R⁵; said alkyl, cycloalkyl, alkoxy and alkylthio groups being optionally further substituted by one or more fluoro atoms;
each R⁴, R⁵, R⁶, R⁷, R¹² and R¹³ independently represents H or C1 to 6 alkyl;
each R⁸, R⁹, R¹⁰ and R¹¹ independently represents H or C1 to 6 alkyl; or the group NR⁸R⁹ or NR¹⁰R¹¹ together represents a saturated 5- or 6-membered azacyclic ring optionally including one further heteroatom selected from O, S and N, and optionally being substituted by one or more C1 to 6 alkyl groups;
and pharmaceutically acceptable salts thereof; with the proviso that the following compounds are excluded:
5-[(2-chlorophenyl)methyl]-2,4-dihydro-4-phenyl-3H-1,2,4-triazole-3-thione;
5-[(2-chloro-6-fluorophenyl)methyl]-2,4-dihydro-4-phenyl-3H-1,2,4-triazole-3-thione.

27. A method of treating multiple sclerosis in a subject comprising administering to such subject an effective amount of a compound selected from:
5-(2,5-dimethoxybenzyl)-4-[3-(methylthio)phenyl]-2,4-dihydro-[1,2,4]triazole-3-thione;
5-(2,5-dimethoxybenzyl)-4-(4-iodophenyl)-2,4-dihydro-[1,2,4]triazole-3-thione;
4-(4-carboxyphenyl)-5-(2,4,6-trimethylbenzyl)-2,4-dihydro-[1,2,4]triazole-3-thione;
5-(2,5-dimethoxybenzyl)-4-[4-(piperidinosulfonyl)phenyl]-2,4-dihydro-[1,2,4]triazole-3-thione;
5-(2,4,6-trimethylbenzyl)-4-(4-sulfamoylphenyl)-2,4-dihydro-[1,2,4]triazole-3-thione;
4-[2-chloro-5-(trifluoromethyl)phenyl]-5-(2,5-dimethoxybenzyl)-2,4-dihydro-[1,2,4]triazole-3-thione;
5-(2-bromobenzyl)-4-(4-sulfamoylphenyl)-2,4-dihydro-[1,2,4]triazole-3-thione;
5-(2,5-dimethoxybenzyl)-4-(3,4,5-trimethoxyphenyl)-2,4-dihydro-[1,2,4]triazole-3-thione;
5-(2-chlorobenzyl)-4-(2-ethoxyphenyl)-2,4-dihydro-[1,2,4]triazole-3-thione;
5-(2-chlorobenzyl)-4-(3-acetylphenyl)-2,4-dihydro-[1,2,4]triazole-3-thione;
5-(2-chlorobenzyl)-4-(4-fluorophenyl)-2,4-dihydro-[1,2,4]triazole-3-thione;
5-(2-chlorobenzyl)-4-(2-methoxy-5-methylphenyl)-2,4-dihydro-[1,2,4]triazole-3-thione;
5-(2-chlorobenzyl)-4-(-3-methoxycarbonyl)phenyl-2,4-dihydro-[1,2,4]triazole-3-thione;
5-(2-chlorobenzyl)-4-(2-methoxyphenyl)-2,4-dihydro-[1,2,4]triazole-3-thione;
5-(2-chlorobenzyl)-4-(3-methylphenyl)-2,4-dihydro-[1,2,4]triazole-3-thione;
5-(2-chlorobenzyl)-4-(3-methoxyphenyl)-2,4-dihydro-[1,2,4]triazole-3-thione;
5-(2-chlorobenzyl)-4-(2-methylphenyl)-2,4-dihydro- [1,2,4]triazole-3-thione;
5-(2-chlorobenzyl)-4-(3-hydroxymethylphenyl)-2,4-dihydro-[1,2,4]triazole-3-thione;
5-(2-chlorobenzyl)-4-(3-chlorophenyl)-2,4-dihydro-[1,2,4]triazole-3-thione;
5-(2-fluorobenzyl)-4-phenyl-2,4-dihydro-[1,2,4]triazole-3-thione;
5-(2-bromo-5-methylbenzyl)-4-phenyl-2,4-dihydro-[1,2,4]triazole-3-thione;
5-(2-bromobenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(2-chlorobenzyl)-4-(4-methylphenyl)-2,4-dihydro-[1,2,4]triazole-3-thione;
5-(2,3-dichlorobenzyl)-4-phenyl-2,4-dihydro-[1,2,4]triazole-3-thione;
5-(2-methylbenzyl)-4-(2-methoxyphenyl)-2,4-dihydro-[1,2,4]triazole-3-thione;
5-(2,6-dimethylbenzyl)-4-phenyl-2,4-dihydro-[1,2,4]triazole-3-thione;
5-(2-methylbenzyl)-4-phenyl-2,4-dihydro-[1,2,4]triazole-3-thione;
5-(2-chlorobenzyl)-4-(4-chlorophenyl)-2,4-dihydro-[1,2,4]triazole-3-thione;
5-(2-chlorobenzyl)-4-(2-chlorophenyl)-2,4-dihydro-[1,2,4]triazole-3-thione;
5-(2-chlorobenzyl)-4-phenyl-2,4-dihydro-[1,2,4]triazole-3-thione;
5-(2-methoxybenzyl)-4-phenyl-2,4-dihydro-[1,2,4]triazole-3-thione;
5-(2-butoxybenzyl)-4-phenyl-2,4-dihydro-[1,2,4]triazole-3-thione;
5-(2-chloro-5-methoxybenzyl)-4-phenyl-2,4-dihydro-[1,2,4]triazole-3-thione;
5-(2-chloro-6-fluoro-3-methylbenzyl)-4-phenyl-2,4-dihydro-[1,2,4]triazole-3-thione;
5-(2,3-dimethoxybenzyl)-4-phenyl-2,4-dihydro-[1,2,4]triazole-3-thione; or
4-phenyl-5-(2,3,4-trimethoxybenzyl)-2,4-dihydro-[1,2,4]-triazole-3-thione;
or a pharmaceutically acceptable salt thereof.

28. A process for the preparation of a compound of formula (Ia), as defined in claim 23, or a pharmaceutically acceptable salt, enantiomer, diastereomer or racemate thereof, which process comprises:
(a) reaction of a thiosemicarbazide derivative of formula (II) 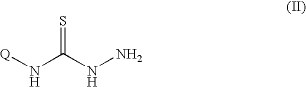
with an ester of formula (III) 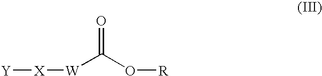
wherein R represents C1 to 6 alkyl; or
(b) reaction of a thiosemicarbazide derivative of formula (II), with a carboxylic acid of formula (IV) 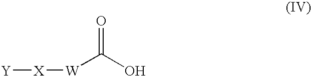
in the presence of a coupling agent; or
(c) reaction of a thiosemicarbazide derivative of formula (II), with an acyl chloride of formula (V) 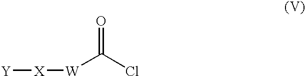
or
(d) reaction of an isothiocyanate derivative of formula (VI) 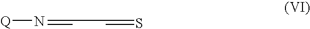
with an acid hydrazide of formula (VII) 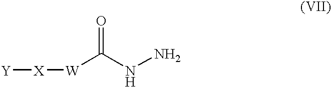
or
(e) reaction of an isocyanate derivative of formula (VIII) 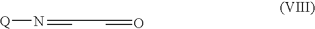
with an acid hydrazide of formula (VII) 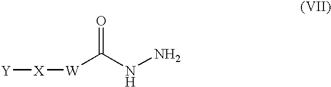
followed by treatment of the intermediate 2,4-dihydro-[1,2,4]triazol-3-one with Lawesson's reagent;
and where necessary converting the resultant compound of formula (I), or another salt thereof, into a pharmaceutically acceptable salt thereof; or converting the resultant compound of formula (I) into a further compound of formula (I); and where desired converting the resultant compound of formula (I) into an optical isomer thereof.
